(19) United States
(12) Patent Application Publication
(10) Pub. No.: US 2015/0203880 A1
(43) Pub. Date: Jul. 23, 2015

(54) CO-CULTURE BASED MODULAR ENGINEERING FOR THE BIOSYNTHESIS OF ISOPRENOIDS, AROMATICS AND AROMATIC-DERIVED COMPOUNDS

(71) Applicant: Massachusetts Institute of Technology, Cambridge, MA (US)

(72) Inventors: GREGORY STEPHANOPOULOS, Winchester, MA (US); Kang Zhou, Revere, MA (US); Kangjian Qiao, Cambridge, MA (US); Steven Edgar, Cambridge, MA (US); Haoran Zhang, Malden, MA (US)

(73) Assignee: Massachusetts Institute of Technology, Cambridge, MA (US)

(21) Appl. No.: 14/534,624

(22) Filed: Nov. 6, 2014

Related U.S. Application Data
(60) Provisional application No. 62/011,653, filed on Jun. 13, 2014.
(60) Provisional application No. 61/900,526, filed on Nov. 6, 2013.

(51) Int. Cl. C12P 13/00; C12P 7/46; C12P 7/44; C12P 7/26; C12P 7/22

(52) U.S. Cl. CPC C12P 13/001; C12P 7/26; C12P 7/22; C12P 7/44; C12P 7/46

(57) ABSTRACT

The invention relates to co-cultures and their use in the biosynthesis of functionalized taxanes, other isoprenoids, aromatics, and aromatic-derived compounds.

### CROSS REFERENCE TO RELATED APPLICATIONS

This application claims the benefit under 35 U.S.C. §119(e) of U.S. provisional application No. 61/900,526, filed Nov. 6, 2013 and U.S. provisional application No. 62/011,653, filed Jun. 13, 2014, each of which are incorporated by reference herein in its entirety.

### GOVERNMENT INTEREST

This invention was made with Government support under Grant No. R01 GM085323 awarded by the National Institutes of Health and under Contract No. DE-AR0000059 awarded by the Department of Energy. The Government has certain rights in the invention.

### FIELD OF THE INVENTION

The invention relates to co-cultures and their use in the biosynthesis of compounds, such as isoprenoids (e.g. functionalized taxanes), aromatics and aromatic-derived molecules.

### BACKGROUND OF THE INVENTION

Total synthesis of structurally complex compounds is costly and yields a limited supply of the desired compound. Similarly, cell culture and extraction based systems used for the production of some desired compounds have yielded a limited and variable supply of some compounds. Based on advances in microbial engineering and fermentation technologies, there has been much interest in heterologous production of compounds in microbial culture as it could provide a sustainable and reproducible process for the supply of natural products. However, development of this technology has been impaired by difficulties in reconstituting the biosynthetic pathways, feedback inhibition of the pathways, and generation of toxic metabolic byproducts that limit the efficiency of this approach.

Isoprenoids are a class of natural products produced by plants that includes paclitaxel, a potent antitumor agent, and artemisinic acid, an antimalarial drug. Efforts to improve plant production of desired molecules such as isoprenoids have focused on plant cell-based cultures including culturing Taxus plant cells and the endophytic fungus, Fusarium mairei in bioreactor tanks separated by a membrane (Li, Tao and Cheng, 2009). Though F. mairei is independently capable of producing low levels of isoprenoids, the presence of the fungus stimulated increased production of isoprenoids by the plant cells (to 25.6 mg/L) over the course of 15 days. However, no transfer of paclitaxel intermediates was made between the two cell cultures.

A method for microbial production of methyl halides was recently established (Bayer T. S. et al., 2009) involving the co-culture of Saccharomyces cerevisiae that have been genetically engineered to synthesize methyl halides with a cellulolytic bacterium, Actinotalea fermentans. In this case, A. fermentans degrades cellulose into ethanol and acetate which are then utilized as carbon sources for S. cerevisiae, though the bacterium does not produce methyl halides nor contribute to the precursor molecules. Thus the A. fermentans only provided a carbon source for the S. cerevisiae; A. fermentans did not directly contribute to synthesis of the final product.

Aromatic compounds and aromatic-derived compounds are widely used in modern industry; for example, muconic acid is a precursor for the production of nylon, polyurethane, and polyethylene terephthalate (PET). The vast majority of aromatic and aromatic-derived compounds are produced by the petroleum industry. Due to the increasing global environment, economic and sustainability concerns, there is much interest in alternative methods of production. Microbial production of such compounds in a single cell has been explored but has only resulted in limited production yield.

### SUMMARY OF THE INVENTION

Described herein is the novel concept of reconstituting a heterologous metabolic pathway in a microbial consortium instead of a single microbe. As an exemplary heterologous metabolic pathway, the pathway for oxygenated paclitaxel precursors was used and divided into two modules, each of which was expressed in a different cell type, Escherichia coli and S. cerevisiae. When the two cell types formed a microbial community, i.e., a synthetic cellular consortium, the intermediate (taxadiene) produced by E. coli was translocated into the S. cerevisiae cells, where it was further functionalized to yield 20 mg/L oxygenated taxanes in 90 h. Similar performance was demonstrated in a consortium of two E. coli strains, one engineered to synthesize taxadiene and the other to convert taxadiene to its oxygenated products. In another exemplary heterologous metabolic pathway, a pathway for aromatic compounds or aromatic-derived compounds was divided into two modules, each of which was expressed in a different E. coli strain. When the two E. coli strains formed a synthetic cellular consortium, the intermediate (dehydroshikimate) produced by one E. coli strain was translocated into the other E. coli strain, where it was converted into an aromatic compound or aromatic-derived compound. The methods demonstrated here can improve modularity of microbial metabolite production processes and also fully utilize specialization of different microbes for synthesis of complex natural products.

Aspects of the invention relate to a synthetic cellular consortium including a first organism with a first part of a biosynthetic pathway that produces a first compound and a second organism with a second part of the biosynthetic pathway that is able to convert the first compound into a second compound. In some embodiments, the first and/or second organism is a bacterium. In some embodiments, the bacterium is Escherichia coli, Bacillus subtilis or Bacillus megaterium. In some embodiments, the E. coli, Bacillus subtilis or Bacillus megaterium is genetically engineered.

In some embodiments, the first organism recombinantly expresses one or more enzymes of a biosynthetic pathway. In some embodiments, the biosynthetic pathway is a secondary metabolite biosynthetic pathway. In some embodiments, the secondary metabolite biosynthetic pathway is an isoprenoid biosynthetic pathway. In some embodiments, the biosynthetic pathway is a 2-C-methyl-D-erythritol 4-phosphate/1-deoxy-D-xylulose 5-phosphate (MEP) pathway. In some embodiments, the first organism recombinantly expresses any of the genes dxs, idi, ispD, ispF of the MEP pathway. In some embodiments, the first organism recombinantly expresses any of the genes ispG and ispH of the MEP pathway. In some embodiments, the genes of the MEP pathway are isolated from E. coli.

In some embodiments of the invention, the first organism recombinantly expresses geranylgeranyl diphosphate synthase (GGPPS). In some embodiments, a nucleic acid encoding GGPPS is isolated from T. canadensis. In some embodiments, the first organism recombinantly expresses taxadiene synthase (TS). In some embodiments, a nucleic acid encoding TS is isolated from T. brevifolia.

In some embodiments, one or more of the nucleic acids encoding enzymes of the MEP pathway, GGPPS or TS are integrated into the genome at a specific site. In some embodiments, one of more of the nucleic acids encoding enzymes of the MEP pathway, GGPPS or TS is on a plasmid. In some embodiments, expression of one or more of the nucleic acids is under control of a constitutively active promoter. In some embodiments, the promoter is the bacteriophage T7 promoter.

In other embodiments, the biosynthetic pathway is the shikimate pathway. In some embodiments, the genes ydiB and/or aroE are mutated or deleted from the first organism. In some embodiments, the first organism expresses one or more global transcription machinery genes. In some embodiments, the global transcription machinery gene is rpoA. In some embodiments, the sequence of rpoA comprises one or more mutations.

In some embodiments, one of more of the nucleic acid encoding genes are codon optimized for expression in E. coli. In some embodiments, genes encoding F₁F₀⁺-ATP synthase subunits are mutated or deleted from the first organism. In some embodiments, the genes encoding F₁F₀⁺-ATP synthase subunits that are mutated or deleted are atpFH.

In some embodiments, the first and/or second organism is a yeast. In some embodiments, the yeast is Saccharomyces cerevisiae, Yarrowia lipolytica, or Pichia pastoris. In some embodiments, the S. cerevisiae, Yarrowia lipolytica or Pichia pastoris is genetically engineered.

In some embodiments, the first and/or second organism is a plant cell. In some embodiments, the plant cell belongs to the genus Taxus. In some embodiments, the Taxus cell is induced with methyl jasmonate. In some embodiments, the Taxus cell is genetically engineered.

In some embodiments, the second organism recombinantly expresses one or more enzymes of a biosynthetic pathway. In some embodiments, the second organism recombinantly expresses components of an oxidoreductase, components of an acyltransferase or an enzyme catalyzing hydroxylation.

In some embodiments, the biosynthetic pathway is a secondary metabolite biosynthetic pathway. In some embodiments, the secondary metabolite biosynthetic pathway is an isoprenoid biosynthetic pathway, a polyketide biosynthetic pathway or an alkaloid biosynthetic pathway.

In some embodiments, the second organism recombinantly expresses components of a cytochrome P450. In some embodiments, the second organism recombinantly expresses taxadiene 5α hydroxylase and NADPH-cytochrome P450 reductase. In some embodiments, the second organism recombinantly expresses taxadiene 5α hydroxylase and NADPH-cytochrome P450 reductase as a single polypeptide. In some embodiments, a nucleic acid encoding taxadiene 5α hydroxylase and/or NADPH-cytochrome P450 reductase is isolated from T. cuspidata.

In some embodiments, a nucleic acid encoding taxadiene 5α hydroxylase and NADPH-cytochrome P450 reductase is integrated into the genome at a specific site. In some embodiments, a nucleic acid encoding taxadiene 5α hydroxylase and NADPH-cytochrome P450 reductase is on a plasmid. In some embodiments, expression of the nucleic acid encoding taxadiene 5α hydroxylase and NADPH-cytochrome P450 reductase is driven by a TEF promoter, a UAS-GPD promoter, a GPD promoter, or an ACS promoter.

In some embodiments, the biosynthetic pathway is for the production of an aromatic compound or an aromatic-derived compound. In some embodiments, the aromatic-derived compound is cis, cis-muconic acid (muconic acid). In some embodiments, the aromatic compound is 3-aminobenzoate. In some embodiments, the aromatic compound is p-hydroxybenzoate (PHB).

In some embodiments, the second organism recombinantly expresses one or more of the genes aroE, ydiB, aroL, aroA, aroC, and ubiC of the PHB biosynthetic pathway. In some embodiments, the second organism recombinantly expresses pctV for the biosynthesis of 3-aminobenzoate. In some embodiments, the second organism further recombinantly expresses shiA.

In some embodiments, a carbon source utilized by the first organism comprises xylose, glucose and/or glycerol. In some embodiments, the second organism can utilize a carbon metabolic byproduct produced by the first organism. In some embodiments, the carbon metabolic byproduct produced by the first organism is acetate. In some embodiments, a carbon source utilized by the second organism comprises xylose, glucose, and/or glycerol. In some embodiments, the carbon source utilized by the first organism is a different carbon source than the carbon source utilized by the second carbon source.

In some embodiments, the first compound produced by the first organism comprises at least part of the second compound produced by the second organism. In some embodiments, the first compound produced by the first organism is membrane permeable or transported out of the first organism. In some embodiments, the first compound produced by the first organism is an intermediate of the isoprenoid pathway. In some embodiments, the isoprenoid intermediate is taxadiene or an oxygenated taxane. In some embodiments, the oxygenated taxane is taxadien-5a-ol, taxadien-5a-ol-10b-ol or taxadiene-5a-acetate-10b-ol. In some embodiments, the second organism converts the isoprenoid intermediate produced by the first organism into an oxygenated taxane or acetylated taxane.

Some aspects of the invention relate to a synthetic cellular consortium that further includes a third organism that converts the second compound into a third compound.

In some embodiments, the first compound produced by the first organism is an intermediate of the shikimate pathway. In some embodiments, the intermediate of the shikimate pathway is dehydroshikimate (DHS). In some embodiments, the second organism converts DHS produced by the first organism into an aromatic compound or an aromatic-derived compound. In some embodiments, the aromatic-derived compound is muconic acid. In some embodiments, the aromatic compound is p-hydroxybenzoate. In some embodiments, the aromatic compound is 3-aminobenzoate.

Aspects of the invention relate to a method of synthesizing a compound involving culturing the synthetic microbial consortium described herein. In some embodiments, the synthetic cellular consortium is cultured in a bioreactor or a shake flask. In some embodiments, the method further involves isolating or purifying the second compound. In some embodiments, the second compound is an oxygenated taxane or acetylated taxane. In some embodiments, a supernatant of the culture comprises 20-25000 mg/L oxygenated taxanes.

In some embodiments, the second compound is an aromatic compound or an aromatic-derived compound. In some embodiments, the aromatic-derived compound is muconic acid. In some embodiments, the supernatant of the culture comprises at least 400 mg/L muconic acid. In some embodiments, the aromatic compound is PHB or 3-aminobenzoate. In some embodiments, the supernatant of the culture comprises at least 50 mg/L PHB. In some embodiments, the supernatant of the culture comprises at least 3 mg/L 3-aminobenzoate.

Some aspects of the invention relate to a culture comprising the synthetic cellular consortium described herein.

Aspects of the invention relate to a method of synthesizing a compound involving culturing cells of a first organism with a first part of a biosynthetic pathway that produces a first compound, isolating the first compound from the culture of the first organism, separately culturing cells of a second organism with a second part of the biosynthetic pathway that converts the first compound into a second compound, and adding the isolated first compound to the culture of the second organism. In some embodiments, the method further involves isolating the second compound from the culture of the second organism.

In some embodiments, the first and/or second organism is a bacterium. In some embodiments, the bacterium is Escherichia coli, Bacillus subtilis or Bacillus megaterium. In some embodiments, the Escherichia coli, Bacillus subtilis or Bacillus megaterium is genetically engineered. In some embodiments, the E. coli is an E. coli K12 derivative or an E. coli B derivative.

In some embodiments, the first organism recombinantly expresses one or more enzymes of a biosynthetic pathway. In some embodiments, the biosynthetic pathway is a secondary biosynthetic pathway. In some embodiments, the secondary biosynthetic pathway is an isoprenoid biosynthetic pathway. In some embodiments, the biosynthetic pathway is the MEP pathway. In some embodiments, the first organism recombinantly expresses the genes dxs, idi, ispD, ispF of the MEP pathway. In some embodiments, the first organism recombinantly expresses any of the genes ispG and ispH of the MEP pathway. In some embodiments, the genes of the MEP pathway are isolated from E. coli.

In some embodiments, the first organism recombinantly expresses geranylgeranyl diphosphate synthase (GGPPS). In some embodiments, a nucleic acid encoding GGPPS is isolated from T. canadensis. In some embodiments, the first organism recombinantly expresses taxadiene synthase (TS). In some embodiments, a nucleic acid encoding TS is isolated from T. brevifolia. In some embodiments, one or more of the nucleic acids encoding enzymes of the MEP pathway, GGPPS or TS is integrated into the genome at a specific site. In some embodiments, one or more of the nucleic acids encoding enzymes of the MEP pathway, GGPPS or TS is on a plasmid.

In some embodiments, the expression of one or more of the nucleic acids is under control of a constitutively active promoter. In some embodiments, the promoter is the bacteriophage T7 promoter.

In some embodiments, the biosynthetic pathway is the shikimate pathway. In some embodiments, the genes ydiB and/or aroE are mutated or deleted from the first organism. In some embodiments, the first organism expresses one or more global transcription machinery genes.

In some embodiments, any or all of the nucleic acid encoding genes are codon optimized for expression in E. coli.

In some embodiments, the first and/or second organism is a yeast. In some embodiments, the yeast is Saccharomyces cerevisiae, Yarrowia lipolytica, or Pichia pastoris. In some embodiments, the S. cerevisiae, Yarrowia lipolytica, or Pichia pastoris is genetically engineered.

In some embodiments, the first and/or second organism is a plant cell. In some embodiments, the plant cell belongs to the genus Taxus. In some embodiments, the Taxus cell is induced with methyl jasmonate. In some embodiments, the Taxus cell is genetically engineered.

In some embodiments, the second organism recombinantly expresses one or more enzymes of a biosynthetic pathway. In some embodiments, the biosynthetic pathway is a secondary biosynthetic pathway. In some embodiments, the secondary metabolite biosynthetic pathway is an isoprenoid biosynthetic pathway, an polyketide biosynthetic pathway or an alkaloid biosynthetic pathway.

In some embodiments, the second organism recombinantly expresses components of an oxidoreductase, an acyltransferase or an enzyme catalyzing hydroxylation. In some embodiments, the second organism recombinantly expresses components of a cytochrome P450. In some embodiments, the second organism recombinantly expresses taxadiene 5α hydroxylase and NADPH-cytochrome P450 reductase. In some embodiments, the second organism recombinantly expresses taxadiene 5α hydroxylase and NADPH-cytochrome P450 reductase as a single polypeptide.

In some embodiments, the second organism recombinantly expresses taxadiene 5α hydroxylase and NADPH-cytochrome P450 reductase with N-terminal membrane-binding domains. In some embodiments, a nucleic acid encoding taxadiene 5α hydroxylase and NADPH-cytochrome P450 reductase is isolated from T. cuspidata. In some embodiments, a nucleic acid encoding taxadiene 5α hydroxylase and NADPH-cytochrome P450 reductase is integrated into the genome at a specific site. In some embodiments, a nucleic acid encoding taxadiene 5α hydroxylase and NADPH-cytochrome P450 reductase is on a plasmid. In some embodiments, expression of the nucleic acid encoding taxadiene 5α hydroxylase and NADPH-cytochrome P450 reductase is driven by a TEF promoter, an UAS-GPD promoter, a GPD promoter, or an ACS promoter.

In some embodiments, the biosynthetic pathway is for the production of an aromatic compound or an aromatic-derived compound. In some embodiments, the aromatic-derived compound is muconic acid. In some embodiments, the aromatic compound is 3-aminobenzoate. In some embodiments, the aromatic compound is p-hydroxybenzoate (PHB). In some embodiments, the second organism recombinantly expresses one or more of the genes aroZ, aroY, and catA of a muconic acid biosynthetic pathway. In some embodiments, the second organism recombinantly expresses one or more of the genes aroE, ydiB, aroL, aroA, aroC, and ubiC of the PHB biosynthetic pathway. In some embodiments, the second organism recombinantly expresses pctV for the biosynthesis of 3-aminobenzoate. In some embodiments, the second organism further recombinantly expresses shiA.

In some embodiments, the first compound produced by the first organism comprises at least part of the second compound produced by the second organism. In some embodiments, the first compound produced by the first organism is membrane permeable or transported out of the first organism. In some embodiments, the intermediate/first compound produced by the first organism is an intermediate of the isoprenoid pathway. In some embodiments, the isoprenoid intermediate is taxadiene or an oxygenated taxane. In some embodiments, the oxygenated taxane is taxadien-5a-ol, taxadien-5a-ol-10b-ol or taxadien-5a-acetate-10b-ol.

In some embodiments, the second organism converts the isoprenoid intermediate produced by the first organism into an oxygenated taxane or acetylated taxane.

In some embodiments, the first compound produced by the first organism is an intermediate of the shikimate pathway. In some embodiments, the intermediate of the shikimate pathway is dehydroshikimate (DHS). In some embodiments, the second organism converts DHS produced by the first organism into an aromatic compound or an aromatic-derived compound. In some embodiments, the second organism converts DHS produced by the first organism into muconic acid. the second organism converts DHS produced by the first organism into p-hydroxybenzoate. the second organism converts DHS produced by the first organism into 3-aminobenzoate.

In some embodiments, the method further involves isolating or purifying the second compound.

Aspects of the invention relate to recombinant cells that express a DHS dehydratase (aroZ), a protocatechuic acid (PCA) decarboxylase (aroY), and a catechol 1,2-dioxygenase (catA), and in which the genes ydiB and aroE have been mutated or deleted. Other aspects of the invention relate to recombinant cells that express a shikimate dehydrogenase (aroE), a shikimate kinase (aroL), a 5-enolpyruvyl shikimate 3-phosphate synthase (aroA), a chorismate synthase (aroC), and a chorismate pyruvate lyase (ubiC). Other aspects of the invention relate to recombinant cells that express an aminotransferase (pctV) and in which the genes ydiB and aroE have been mutated or deleted.

In some embodiments, the cell further expresses one or more global transcription machinery genes. In some embodiments, the global transcription machinery gene is rpoA. In some embodiments, the sequence of rpoA comprises one or more mutations. In some embodiments, the cell further expresses a shikimate/DHS transporter (shiA).

In some embodiments, the cell is a microbial cell. In some embodiments, the microbial cell is an Escherichia coli cell. In some embodiments, the Escherichia coli cell is an Escherichia coliBL21 (DE3) cell.

Some aspects of the invention relate to methods of producing muconic acid comprising culturing any of the cells described herein to produce muconic acid. In some embodiments, the method further comprises isolating and/or purifying the muconic acid.

Some aspects of the invention relate to methods of producing p-hydroxybenzoate (PHB) comprising culturing any of the cells described herein to produce PHB. In some embodiments, the method further comprises isolating and/or purifying the PHB.

Other aspects of the invention relate to methods of producing 3-aminobenzoate comprising culturing any of the cells described herein to produce 3-aminobenzoate. In some embodiments, the method further comprises isolating and/or purifying the 3-aminobenzoate.

These and other aspects of the invention, as well as various embodiments thereof, will become more apparent in reference to the drawings and detailed description of the invention.

### BRIEF DESCRIPTION OF THE DRAWINGS

FIGS. 1A and 1B show a schematic representation of a synthetic microbial consortium comprising E. coli and S. cerevisiae cooperating synergistically at two levels. FIG. 1A shows synthesis of oxygenated taxanes, and FIG. 1B shows cell growth. E. coli uses xylose as substrate producing acetate, which, in turn, is used by S. cerevisiae without producing ethanol as byproduct. This mutualistic interaction minimizes E. coli inhibition by acetate and ethanol, normally produced when grown on glucose. The arrows with solid lines indicate biomass and compounds derived from xylose. The arrows with dotted lines indicate acetate derivatives.

FIGS. 2A-2D show co-culture of E. coli and S. cerevisiae for production of oxygenated taxanes in glucose medium. FIG. 2A depicts oxygenated taxane production by the co-culture in glucose medium. FIG. 2B shows a significant decrease in titer of total taxanes produced in the presence of S. cerevisiae. FIG. 2C shows ethanol secretion was significantly elevated in the co-culture system, which was hypothesized to have caused the drastic reduction in taxane production. FIG. 2D confirms the inhibition of E. coli by ethanol by quantifying the E. coli cell number, which was also significantly decreased in presence of S. cerevisiae. Error bars indicate standard error (n=3). Data labeled with “E. C.” corresponds to E. coli mono-culture; and data labeled with “Co” corresponds to the co-culture system.

FIGS. 3A-3E demonstrate cooperative co-culture of E. coli and S. cerevisiae for the production of oxygenated taxanes in xylose medium. FIG. 3A shows that in xylose-limiting medium S. cerevisiae can only grow in the presence of E. coli as S. cerevisiae cannot metabolize xylose. FIG. 3B demonstrates that extracellular acetate concentrations are significantly reduced by the presence of S. cerevisiae, indicating that S. cerevisiae grows on acetate. FIG. 3C shows production of taxanes by the E. coli mono-culture was virtually unchanged by the presence of the S. cerevisiae. FIG. 3D shows no oxygenated taxanes were produced by single microbial culture. FIG. 3E shows 20 mg/L oxygenated taxanes were produced by the co-culture in 90 h after bioreactor optimization. Error bars indicate standard error (n=2). Data labeled “E.C.” correspond to E. coli mono-cultures. Data labeled “S.C.” correspond to S. cerevisiae monocultures. Data labeled “Co” correspond to the co-culture system.

FIG. 4A schematically presents a model synthetic microbial consortium comprising two E. coli strains that cooperatively synthesize oxygenated taxanes. FIG. 4B shows 0.8 mg/L oxygenated taxanes were produced by the E. coli-E. coli consortium in fed-batch bioreactor, whereas no oxygenated taxanes were produced by culture of any single E. coli strain (data not shown). Data are labeled to indicate oxygenated taxane concentration taxadiene production. Error bars indicate standard error (SD=2).

FIG. 5 schematically presents construction of the S. cerevisiae strain expressing taxadiene 5α-hydroxylase and its reductase. P_{TEF}: TEF promoter; T_{CYC}: CYC terminator; 5αCYP: taxadiene 5α-hydroxylase (a CYP); CPR: CYP reductase; URA: uracil marker; linker sequence GSTST: SEQ ID NO:105.

FIG. 6 shows taxadiene oxygenation by the strain S. cerevisiae BY4700_{—}5aCYPCPR. 1 mL of BY4700_{—}5aCYPCPR culture was inoculated into 28 mL YPD medium supplemented with 12 mg/L taxadiene. The cell culture was incubated at 22° C./250 rpm and was sampled at the indicated time points. The results show that taxadiene (circles) was efficiently converted to oxygenated taxanes (squares) by this strain. No oxygenated taxane was produced in the control experiment where this strain was replaced by wild type S. cerevisiae BY4700 (data not shown). Error bars indicate standard error (n=2).

FIG. 7 shows the S. cerevisiae strain expressing taxadiene 5α-hydroxylase and its reductase is unable to produce taxadiene (circles) nor oxygenated taxanes (squares) without co-culture with the taxadiene-producing E. coli. Additionally, the experiment also shows that the S. cerevisiae cannot metabolize xylose (diamonds) without E. coli. Ethanol concentration was also measured (triangles).

FIG. 8A shows the effect of ethanol on growth of E. coli MG1655_MEP_TG. FIG. 8B shows the effect of ethanol on taxadiene production by E. coli MG1655_MEP_TG. 50 g/L ethanol was added to culture of the taxadiene-producing E. coli in shake flask (+EtOH). Ethanol repressed both E. coli growth and taxadiene production compared to cultures that did not receive exogenous ethanol (−EtOH). Error bars indicate standard error (n=2).

FIGS. 9A-9C show the identification of oxygenated taxanes produced by the microbial consortia. FIG. 9A depicts ion chromatography traces (288 m/z, characteristic m/z of mono-hydroxylated taxadiene) that identified four oxygenated taxanes (X1-X4) in extracts from an E. coli-S. cerevisiae co-culture system. None of these peaks were detected in single culture of taxadiene-producing E. coli MG1655_MEP_TG nor in single culture of S. cerevisiae BY4700_{—}5aCYPCPR. All of the peaks were detected in single culture of S. cerevisiae BY4700_{—}5aCYPCPR which was supplemented with synthetic taxadiene, indicating that compounds X1-X4 were derived from taxadiene. FIGS. 9B-9C show mass spectra of each of the compounds X1-X4 in cell extracts.

FIG. 10 shows validation of a centrifugation protocol for estimating the cell density of S. cerevisiae in an E. coli-S. cerevisiae co-culture. 200 uL of E. coli or S. cerevisiae cell suspension was centrifuged at 100 rpm for 1 min (Beckman coulter microfuge 18). The supernatant was removed and the pellets were resuspended in 200 uL water. Optical density at 600 nm for the cell suspension before centrifugation (black bars) and of the cells resuspended in water (white bars) was measured. The results show that all S. cerevisiae cells were collected in pellets while E. coli cells cannot be pelleted by this protocol. Therefore, this centrifugation protocol can selectively separate S. cerevisiae from an E. coli-S. cerevisiae mixture for cell density determination. Error bars indicate standard error (n=3).

FIG. 11 shows a schematic representation of a synthetic cellular consortium comprising E. coli and T. chinensis cells. E. coli cells efficiently produce taxadiene and T. chinensis cells induced with methyl jasmonate efficiently convert taxadiene into Baccatin III and Taxol.

FIG. 12 shows a schematic representation of an alternative co-culture method in which E. coli and T. chinensis cells are cultured separately. E. coli cells efficiently produce taxadiene, which is isolated and flash purified from the culture of E. coli cells. The taxadiene from E. coli fermentation is then added to the culture of T. chinensis cells, which efficiently convert taxadiene into Baccatin III and Taxol.

FIG. 13 shows process engineering of the system can result in increased oxygenated taxane titer. The amount of S. cerevisiae used to inoculate the co-culture was increased and additional nutrients were supplied at 41 hours (circles). This optimization resulted in 3-fold increased production of oxygenated taxanes compared to a control co-culture (squares).

FIGS. 14A-14C present optimization of the recombinant expression systems of S. cerevisiae and the effect on oxygenated taxane production. FIG. 14A demonstrates that replacing the TEF promoter (TEFp) with other promoters affects oxygenated taxane production; the other promoters used included UAS-GPDp, GPDp, ACSp. FIG. 14B shows oxygenated taxane production in co-cultures of E. coli with either S. cerevisiae with the TEFp or with the best promoter from FIG. 14A (UAS-GPDp). FIG. 14C presents the relative amounts of taxadiene and oxygenated taxanes produced by the co-culture of E. coli and S. cerevisiae with UAS-GDPp.

FIGS. 15A-15B show genetic engineering of E. coli can affect oxygenated taxane production of the co-culture system. FIG. 15A shows overproduction of acetate by deletion of E. coli genes atpFH (black bars) results in improved S. cerevisiae growth in the co-culture compared to co-culture with E. coli with atpFH intact (white bars). FIG. 15B presents the relative amount of taxadiene and oxygenated taxanes produced by the co-culture of E. coli ΔatpFH and S. cerevisiae.

FIGS. 16A-16F present muconic acid biosynthetic gene functionality assays. FIG. 16A shows a schematic representation of a cell that is engineered to express aroZ and can convert DHS (dehydroshikimate) into protocatechuic acid. FIG. 16B shows a schematic representation of a cell that is engineered to express aroY and can convert protocatechuic acid into catechol. FIG. 16C shows a schematic representation of a cell that is engineered to express catA and can convert catechol into muconic acid. FIG. 16D shows a representative LC-MS trace indicating production of protocatechuic acid by the cell depicted in FIG. 16A. FIG. 16E shows a representative HPLC trace indicating production of catechol by the cell of FIG. 16B. FIG. 16F shows a representative HPLC trace indicating production of muconic acid by the cell of FIG. 16F.

FIG. 17 presents a schematic representation of the engineered pathways for the production of aromatic and aromatic-derived compounds, such as 3-aminobenzoate and muconic acid, using the shikimate pathway intermediate DHS as a substrate. Genes involved in a pathway competing for DHS substrate (ydiB and aroE) are not expressed, as indicated by an “X.”

FIG. 18 presents a schematic representation of recombinant expression of the muconic acid biosynthetic pathway.

FIGS. 19A-B present schematic representation of the DHS flux across the cell membrane. FIG. 19A shows a cell in which DHS is transported out of the cell into the extracellular environment and minimal transport of DHS into the cell. FIG. 19B shows a cell that has been engineered to express the ShiA transporter that imports DHS from the extracellular environment.

FIG. 20 shows the shikimate transporter, ShiA, can also transport DHS. Cells that are deficient for both aroD and shiA are unable to grow, indicated by a “−”, in the absence of DHS. Expression of shiA from a plasmid rescues growth of the cells, indicated by a “+”.

FIG. 21 presents production of muconic acid (MA), catechol (CA), and protocatechuic acid (PCA) and accumulation of dehydroshikimate (DHS) from different engineered E. coli strains. Strain KM is a wild-type E. coli strain that expresses aroY, aroZ, and catA genes. Strain P5g is derived from a tyrosine overproducing strain rpoA14 (Santos et al., 2012) but does not express ydiB and aroE. Strain P5g also expresses aroY, aroZ, and catA genes and carries a global transcription machinery engineering plasmid encoding a mutated rpoA. Strain P5s is derived from the P2g strain and also carries an over-expression plasmid encoding the E. coli ShiA transporter. All three strains contain the plasmid-borne heterologous aroY, aroZ, and catA genes for muconic acid biosynthesis. MA, CA, PCA and DHS are shown left to right in each group of bars. Error bars indicated the standard deviation.

FIG. 22 shows production of muconic acid (MA), catechol (CA), protocatechuic acid (PCA) and dehydroshikimate (DHS) by different E. coli strains, including E. coli K12, BL21 (DE3), and BL21 (DE3) expressing ShiA. All three strains also contain the plasmid-borne heterologous aroY, aroZ, and catA genes for muconic acid biosynthesis and were provided 2 g/L DHS in the culture medium for conversion. MA, CA, PCA and DHS are shown left to right in each group of bars. Error bars indicated the standard deviation.

FIGS. 23A-23B show a co-culture system that uses a second cell to improve DHS utilization. FIG. 23A presents a schematic representation of a single cell recombinant expression system that can be improved by the addition of second cell (BLS) that is able to import and convert DHS into muconic acid. FIG. 23B shows the production of muconic acid (MA), catechol (CA), protocatechuic acid (PCA) and dehydroshikimate (DHS) in monocultures of either P5S or BLS cells, and synthetic consortia of these cells at various ratios of P5S:BLS. MA, CA, PCA and DHS are shown left to right in each group of bars. Error bars indicated the standard deviation.

FIGS. 24A-24C show engineering of a co-culture system for the production of muconic acid. FIG. 24A presents a schematic representation of the muconic acid biosynthetic pathway expressed in a single cell. FIG. 24B presents a schematic representation of the muconic acid biosynthetic pathway expressed in two modules in two cells. The first cell (strain P5.2) expresses rpoA and converts glycerol into DHS. The second cell (strain BLS2) expresses genes for the uptake and conversion of DHS to muconic acid. FIG. 24C shows optimization of muconic acid production by altering the ratio of the first strain (P5.2) to the second strain (BLS2) in the synthetic consortium. Error bars indicate the standard deviation. MA, muconic acid; CA, catechol; PCA, protocatechuic acid; and DHS, dehydroshikimate. MA, CA, PCA and DHS are shown left to right in each group of bars.

FIGS. 25A-25B show differential sugar utilization by each of strains of a synthetic consortium for the production of muconic acid. FIG. 25A shows a schematic representation in which the first strain (P6.2) has been engineered to lack the glucose import system but utilizes xylose to produce DHS. The second strain (BLC) has been engineered to disrupt the xylose utilization pathway but utilizes glucose to convert DHS into muconic acid. FIG. 25B shows optimization of muconic acid production by altering the ratio of the first strain (P6.2) to the second strain (BLC) of the synthetic consortium when grown on a mixture of xylose and glucose. MA, muconic acid; CA, catechol; PCA, protocatechuic acid; and DHS, dehydroshikimate. MA, CA, PCA and DHS are shown left to right in each group of bars. Error bars indicated the standard deviation. Error bars indicate the standard deviation.

FIGS. 26A-26C show engineering of a co-culture system for the production of p-hydroxybenzoate (PHB). FIG. 26A presents a schematic representation of the PHB biosynthetic pathway expressed in a single cell. FIG. 26B presents a schematic representation of the PHB biosynthetic pathway expressed in two modules in two cells. The first cell (strain P5.2) converts glycerol into DHS. The second cell (strain BH2.2) expresses genes for the uptake and conversion of DHS to PHB (ELACU: aroE, aroL, aroA, aroC, and ubiC). FIG. 26C shows optimization of PHB production by altering the ratio of the first strain (P5.2) to the second strain (BH2.2) in the synthetic consortium. PHB, chorismate and shikimate are shown left to right in each group of bars. Error bars indicated the standard deviation. Error bars indicate the standard deviation.

FIG. 27 shows a schematic of the co-culture system in which both the E. coli and the yeast grew on glucose. The E. coli produced taxadiene which can diffuse into the yeast, where it is oxygenated. Taxadiene and oxygenated taxanes are derived from the glucose utilized by the E. coli; ethanol is derived from the glucose utilized by the yeast.

FIG. 28 shows a schematic of the mutualistic E. coli-S. cerevisiae consortium for production of oxygenated taxanes. E. coli grew on xylose and produced acetate that served as sole carbon source for the yeast to grow. The taxadiene produced by the E. coli was oxygenated in the yeast. All E. coli metabolites/cells are derived from xylose; all the carbons of the yeast were from the acetate.

FIGS. 29A-29C show that optimizing yeast growth and engineering the yeast promoters improved production of the oxygenated taxanes. FIG. 29A shows that growth optimization by increasing the yeast inoculum and feeding additional nutrients (upper line) improved the oxygenated taxanes' production by more than two-fold. FIG. 29B shows the UAS-GPDp promoter, identified by promoter screening, was better for taxadiene oxygenation than the previously used TEFp. FIG. 29C shows the co-culture using UAS-GPDp also produced significantly more oxygenated taxanes than that using TEFp. Error bars represent the standard error (s.e.).

FIGS. 30A and 30B show inactivating oxidative phosphorylation of the E. coli improved production of the oxygenated taxanes. FIG. 30A presents a schematic in which oxidative phosphorylation inactivation of the E. coli forces the production of acetate, which became the major method of generating ATP in the E. coli. FIG. 30B shows the taxadiene oxygenation efficiency was greatly improved when the S. cerevisiae was co-cultured with the acetate-overproducing E. coli. Oxygenation efficiency of the TaxE1-TaxS4 co-culture was ˜40-50% (20 mg/L oxygenated taxanes per 40 mg/L total taxanes), and that of the co-culture using the oxidative phosphorylation deficient E. coli strain (TaxE4-TaxS4 co-culture) was ˜75% (30 mg/L oxygenated taxanes per 40 mg/L total taxanes). Error bars represent the standard error (s.e.). In the left panel (“Control”), the upper line is taxadiene production and the lower line is oxygenated taxane production. In the right panel (“Knockout”), the upper line (>48 h) is oxygenated taxane production and the lower line (>48 h) is taxadiene production.

FIGS. 31A-31C show production of a monoacetylated dioxygenated taxane by the E. coli-S. cerevisiae co-culture. FIG. 31A presents a schematic of the early paclitaxel biosynthetic pathway. FIG. 31B shows the yeast co-expressing 5αCYP-CPR, TAT and 10βCYP-CPR (TaxS6) produced putative taxadien-5α-acetate-10β-ol when co-cultured with a taxadiene-producing E. coli. Extracted ion chromatography (346 m/z, molecular weight of monoacetylated dioxygenated taxane) are shown in this graph. The trace labeled 5αCYP is a TaxE4/TaxS4 co-culture. The trace labeled 5αCYP-TAT-10βCYP is a TaxE4/TaxS6 co-culture. FIG. 31C shows that using a stronger promoter (UASGPDp) to express TAT improved production titer of the monoacetylated dioxygenated taxane. Operating the bioreactor at a carbon limiting (CL) condition further improved the production titer and yield (consumed xylose was reduced by 30%). The culture labeled TEFp-TAT was a TaxE4/TaxS6 co-culture, where expression of TAT was driven by TEFp; the culture labeled UASGPDp-TAT was a TaxE4/TaxS7 co-culture, where UASGPDp was used to express TAT; and the culture labeled UASGPDp-TAT CL was a TaxE4/TaxS7 co-culture at a carbon limiting condition. Error bars represent the standard error (s.e.).

FIGS. 32A-32C show use of the E. coli-S. cerevisiae co-culture for production of other oxygenated taxanes. FIG. 32A presents an illustration of biosynthetic pathways of ferruginol and nootkatone. FIG. 32B shows an E. coli strain that was engineered to produce miltiradiene from xylose (TaxE5); TaxE5 itself cannot produce ferruginol. When this E. coli was co-cultured with a yeast expressing a specific CYP and its reductase (TaxS8), the co-culture produced 18 mg/L ferruginol (upper line, TaxE5+TaxS8). Mass spectrum of the produced ferruginol was identical to the one in the literature (data not shown). FIG. 32C shows an E. coli strain engineered to produce valencene (TaxE6); TaxE6 itself cannot produce any oxygenated valencene. When it was co-cultured with a yeast expressing a specific CYP and its reductase (TaxS9), the co-culture produced 30 mg/L nootkatol and low quantity of nootkatone. When an alcohol dehydrogenase was introduced to TaxS9, the resulting strain (TaxS10) produced 4 mg/L nootkatone in presence of TaxE6. Error bars represent the standard error (s.e.). The upper line left panel of FIG. 32C and middle line right panel of FIG. 32C, TaxE6+TaxS9; the middle line left panel of FIG. 32C and upper line right panel of FIG. 32C, TaxE6+TaxS10.

FIG. 33 presents a schematic of an S. cerevisiae cell in which the 5αCYP and its reductase were expressed as a fusion protein, and their transcription was controlled by the TEF promoter.

FIGS. 34A and 34B show feeding the E. coli-S. cerevisiae co-culture exogenous acetate did not improve production of the oxygenated taxanes. FIG. 34A shows that feeding exogenous acetate led to acetate accumulation. FIG. 34B shows production of oxygenated taxanes was not improved by feeding exogenous acetate as compared to the control (FIG. 29A). Error bars represent the standard error (s.e.). In FIG. 34B, the upper line is taxadiene production and the lower line is oxygenated taxane production.

FIGS. 35A-35C show overexpression of pta neither improved the yeast growth nor the taxadiene oxygenation. FIG. 35A presents a schematic of the major acetate production pathway in E. coli. FIG. 35B shows the effect of the overexpression on the yeast growth. Control (left bar) indicates a TaxE1-TaxS4 co-culture, Pta (right bar) indicates a TaxE2-TaxS4 co-culture. FIG. 35C shows the effect of the overexpression on the taxane production. In the left panel (“Control”), the upper line is taxadiene production and the lower line is oxygenated taxane production. In the right panel (“Knockout”), the upper line (<120 h) is taxadiene production and the lower line (<120 h) is oxygenated taxane production. The E. coli strain overexpressing both pta and ackA did not grow in LB medium at 22° C.). Error bars represent the standard error (s.e.).

FIGS. 36A and 36B show mass spectra of the monoacetylated dioxygenated taxane produced by the E. coli-S. cerevisiae co-culture. FIG. 36A shows the spectrum of the compound that was derived from non-labeled taxadiene. FIG. 36B shows the spectrum of the compound that was derived from uniformly 13C-labeled taxadiene. In the latter case, molecular weight of the compound was increased to 366 from 346, consistent with the fact that twenty 12C atoms were substituted by 13C atoms.

FIG. 37 shows optimization of xylose feeding rate improved the titer of the production of the monoacetylated dioxygenated taxane in co-culture of TaxE4 and TaxS7. Linear feeding of xylose was started at the beginning of day 3, and the volume of the culture was maintained at 500 mL through the experiments. A rate of 10 g/day was found to be optimal. In this case, the xylose concentration in the medium was always below its detection limit (0.1 g/L) after day 3, and the total amount of consumed xylose was 80 g/L. Error bars represent the standard error (s.e.).

FIGS. 38A-38C show the effect of S. cerevisiae on E. coli growth and its xylose consumption. FIG. 38A shows E. coli TaxE4 accumulated a high concentration of acetate in the absence of S. cerevisiae TaxS7, which can eliminate the acetate in co-culture. FIG. 38B shows that after acetate concentration reached 5 g/L, the E. coli mono-culture stopped growing, and the E. coli grew to much higher cell density in the co-culture. FIG. 38C shows that after reaching 5 g/L acetate concentration, the E. coli mono-culture also stopped consuming xylose while E. coli kept consuming xylose in presence of the yeast. Error bars represent the standard error (s.e.).

FIG. 39 shows production of the putative taxadien-5α-acetate-10β-ol by the E. coli-S. cerevisiae co-culture was also improved by inactivation of the oxidative phosphorylation. The control co-culture is a TaxE1-TaxS6 co-culture; the knockout co-culture is a TaxE4-TaxS6 co-culture. Error bars represent the standard error (s.e.).

FIG. 40 shows production of oxygenated taxanes by using a two-stage culture. The taxadiene-producing E. coli and 5αCYP-expressing yeast were cultured separately in the glucose medium for three days, and then mixed to produce oxygenated taxanes. This allowed accumulation of taxadiene in the first phase and efficient oxygenation of the taxadiene in the second phase. Error bars represent the standard error (s.e.).

FIGS. 41A-41D present characterization of the E. coli culture, the S. cerevisiae culture and the co-culture of E. coli and S. cerevisiae in the xylose/ethanol medium. FIG. 41A shows the S. cerevisiae strain could not utilize xylose. FIG. 41B shows the E. coli strain could not utilize ethanol. FIG. 41C shows that only E. coli strain can produce taxadiene. FIG. 41D shows that only the co-culture can produce oxygenated taxanes.

FIGS. 42A-42E show that a stable co-culture of E. coli and S. cerevisiae for production of oxygenated taxanes can be maintained by applying two carbon sources. FIG. 42A is a schematic that shows that in this co-culture, xylose can only be utilized by the E. coli strain and ethanol can only be utilized by the S. cerevisiae strain. Taxadiene produced by the E. coli can be oxygenated when it gets into the yeast. Both cells may produce acetate. FIG. 42B shows production of taxadiene and oxygenated taxanes in the co-culture. FIG. 42C shows xylose consumption in the co-culture. FIG. 42D shows ethanol consumption in the co-culture. Ethanol was periodically added. FIG. 42E shows acetate accumulation in the co-culture. Error bars represent the standard error (s.e.).

FIGS. 43A-43B show the distribution of taxadiene in E. coli, medium and yeast, and effect of taxadiene productivity of E. coli on it. FIG. 43A shows an E. coli strain carrying an unbalanced taxadiene synthetic pathway (TaxE11) was confirmed to produce less taxadiene. The control is a TaxE4 mono-culture in shake flask. p5T7TG: TaxE11 mono-culture in shake flask. FIG. 43B shows the taxadiene distribution in the E. coli and S. cerevisiae co-culture. Control: TaxE4/TaxS7 co-culture; p5T7TG is a TaxE11/TaxS11 co-culture. Taxadiene concentration in the co-culture was significantly reduced when a poor taxadiene producer (E. coli TaxE11) was used. Nevertheless, at all conditions, more than 50% of taxadiene was found to be outside E. coli cells (in medium or yeast), indicating that taxadiene can cross cell membranes efficiently (E. coli has two cell membranes), and thus its mass transfer should not be a limiting step in the isoprenoid production processes. The bars in FIG. 43B are segmented as follows: bottom segment, E. coli; middle segment, medium; top segment, yeast.

FIGS. 44A and 44B present an E. coli-E. coli consortium for production of oxygenated taxanes. FIG. 44A shows a synthetic microbial consortium comprising two E. coli strains that cooperatively synthesize oxygenated taxanes. FIG. 44B shows 0.8 mg/L oxygenated taxanes (lower line) were produced by the E. coli-E. coli consortium in fed-batch bioreactor, whereas no oxygenated taxanes was produced by culture of any single E. coli strain (data not shown). Error bars represent the standard error (s.e.).

FIG. 45 presents a schematic illustration of the yeast genome modification method used in this study. Construction of yeast TaxS1 was demonstrated here, and other yeast strains were constructed similarly. “Up” refers to the upstream homologous sequence of YPRC15. “Down” refers to the downstream homologous sequence of YPRC15.

FIGS. 46A and 46B show the five oxygenated taxanes quantified in this study. FIG. 46A shows samples of E. coli co-culture, yeast culture and co-culture were analyzed by GCMS. Multiple new peaks were identified in the co-culture sample as compared to other samples (total ion chromatography). FIG. 46B shows five of the peaks identified in the co-culture sample should be monooxygenated taxane as they also appeared on extracted ion chromatography—288 m/z (272 (taxadiene)+16 (oxygen)).

FIGS. 47A and 47B present mass spectra of the known oxygenated taxanes produced by the co-culture. FIG. 47A shows the mass spectrum of oxa-cyclotaxane (OCT). FIG. 47B shows the mass spectrum of taxadien-5α-ol.

FIG. 48 presents separation of E. coli from S. cerevisiae by using a sucrose-gradient based centrifugation method. The supernatant after the centrifugation mostly contained E. coli, and the pellets mostly contained S. cerevisiae.

FIGS. 49A-49C show that improving muconic acid production is possible by over-expression of key enzymes for the shikimate pathway in the first organism. FIG. 49A presents a schematic of the metabolic network leading from different carbon substrates to the shikimate pathway. PpsA: phosphoenolpyruvate synthetase; TktA: transketolase; AroG: feedback-resistant 2-dehydro-3-deoxyphosphoheptonate aldolase (reference). FIG. 49B shows muconic acid production by the co-culture systems grown on the sugar medium containing 3.3 g/L xylose and 6.6 g/L glucose. The specified strains were co-cultivated with BLC with the initial mixing ratio of 2:2. P6.2 is the control strain; P6.5 over-expressed PpsA and TktA; P6.6 over-expressed PpsA; P6.7 over-expressed AroG. The specified strains were co-n) cultivated with E. coli BC in the glycerol medium with the initial mixing ratio of 1:1. FIG. 49C shows high cell density co-cultivation of P6.6 and BXC to over-produce muconic acid (MA). Batch mode bioreactor was used to consume 6.6 g/L xylose and 13.4 g/L glucose.

### DETAILED DESCRIPTION OF THE INVENTION

In nature, there are many examples of microbial consortia that can efficiently accomplish chemically difficult processes through division of labor among different species, e.g. cellulose degradation (Agapakis, Boyle and Silver, 2012), whereas examples of synthetic consortia comprising genetically engineered microbes are rare. Theoretically, it would be attractive to use synthetic cellular consortia such as synthetic microbial consortia for production of valuable metabolites, especially those that are structurally very complex. Advantages of using such synthetic consortia would be, (i) segmenting long biosynthetic pathways into multiple integratable parts, each of which can be reconstituted and optimized separately in the corresponding species, (ii) combining advantages of different organisms, (iii) exploring beneficial interactions among consortium members to enhance productivity, (iv) minimizing feedback inhibition through spatial pathway segregation, (v) reducing metabolic stress on each organism of the system, and (vi) the ability to change a single module of the system to produce other compounds that share a common intermediate produced by a first organism.

Described herein are methods and compositions for the production of and use of novel synthetic cellular consortia in which biosynthetic pathways are segmented into at least two independent cells. In exemplary embodiments, enzymes of the terpenoid biosynthetic and functionalization pathways were recombinantly expressed in two or more cells that together form a consortium. In other embodiments, enzymes for the production of aromatic or aromatic-derived compounds (e.g., muconic acid, p-hydroxybenzoate, 3-aminobenzoate, alkaloids, flavonoids,) were recombinantly expressed in two or more cells that together form a consortium. In other embodiments, enzymes for the production of short chain dicarboxylic acids were recombinantly expressed in two or more cells that together form a consortium. In yet other embodiments, enzymes for the production of recombinant proteins were recombinantly expressed in two or more cells that together form a consortium. Significantly, the cells within the consortium may be bacteria, yeast and/or plant cells. A requirement for a successful consortium is that the pathway intermediate, in the examples provided, taxadiene dehydroshikimate (DHS), aromatic amino acids, short chain fatty acids, valencene, and miltiradiene cross cell membranes.

The ability of taxadiene to cross cell membranes was first confirmed in previous studies where organic solvent mixed with E. coli cell culture was found to efficiently extract taxadiene (C20) from the cells in bioreactor (Ajikumar et al., 2010). This property is shared by many isoprenoids ranging from C5 to C40, ranging from isoprene (Xue and Ahring, 2011), to limonene (Alonso-Gutierrez et al., 2013), amorphadiene (Zhou et al., 2013) and canthaxanthin (Doshi et al., 2013). Hence, the synthetic cellular consortia and co-culturing methods disclosed herein are generally applicable to production of most isoprenoids and other types of compounds whose precursors are membrane-permeable. This platform represents a new, surprisingly efficient method for production of terpenoids and other structurally complex molecules.

Similarly, intermediates of the shikimate pathway, such as DHS and shikimate, are also able to cross cell membranes (see, for example, FIG. 21), and production of compounds that utilize DHS or shikimate are compatible with the methods described herein. Aromatic amino acids, such as tyrosine, are able to cross the cell membranes and can be further processed for the production, for example, of alkaloids or flavonoids. Additionally, short chain fatty acids are able to cross the cell membranes for the production of short chain dicarboxylic acids, using the methods described herein.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

A “consortium” refers to a collection of organisms that are involved in a common process or by combining their individual processes achieve a common outcome, which in the examples provided is the biosynthesis of terpenoids, aromatic compounds, and aromatic-derived compounds. “Synthetic” refers to a process that is not occurring in nature, nor occurring by chance. For example, the organisms described herein are intentionally combined and each contributes toward the synthesis of a desired compound. Importantly, each of the organisms of the consortium directly contributes to the production of the final compound. For example, a first organism of a consortium will synthesize a first compound that is an intermediate compound of the pathway to synthesize the final compound. Then, a second organism of the consortium further converts the first compound into a second compound. In some embodiments the second compound is the final compound. In some embodiments the second compound is further converted to a third compound by a third organism of the consortium. The synthetic cellular consortium described herein is not limited to prokaryotic or eukaryotic cells. In some embodiments only prokaryotic cells or only eukaryotic cells are used to produce terpenoid compounds. In some embodiments both prokaryotic and eukaryotic cells are used to produce terpenoid compounds. In some embodiments only prokaryotic cells or only eukaryotic cells are used to produce aromatic compounds or aromatic-derived compounds. In some embodiments both prokaryotic and eukaryotic cells are used to produce aromatic compounds or aromatic-derived compounds. In some embodiments, only prokaryotic cells or only eukaryotic cells are used to produce short chain dicarboxylic acids. In some embodiments both prokaryotic and eukaryotic cells are used to produce short chain dicarboxylic acids. In some embodiments, only prokaryotic cells or only eukaryotic cells are used to produce recombinant proteins. In some embodiments both prokaryotic and eukaryotic cells are used to produce recombinant proteins.

Also described herein are methods of culturing the organisms of the consortium. “Culturing” refers to maintaining the indicated organisms within a nutritive environment. In some embodiments the organisms will be maintained within a shared environment, herein referred to as “co-culturing” and the like. In other embodiments, the organisms are maintained in separate environments. Culturing does not require that the organisms are actively replicating. In some embodiments, the organisms will be actively replicating. In other embodiments, the organisms are metabolically active but are not actively replicating.

Described herein are methods and compositions related to the segmentation of a biosynthetic pathway into two or more distinct cells or species. This allows for further independent optimization of each portion of the pathway as well as avoidance of any feedback inhibition of the pathway, which together can increase production potential. In some embodiments, the enzymes of a first portion of the biosynthetic pathway are expressed in a first organism, such that a first compound that is a membrane-permeable intermediate of the biosynthetic pathway is produced. The first compound is then further converted into a second compound by a second organism that expresses additional enzymes of the biosynthetic pathway. Some biosynthetic pathways are regulated by negative feedback such that the presence of an intermediate or the final product of the pathway inhibits expression or activity of enzymes in the first portion of the pathway. This negative feedback mechanism reduces the performance of the pathway and reduces production of the final compound. Segmenting the pathway into two or more distinct cells eliminates the ability of a final compound to inhibit the first portion of the pathway. In some embodiments, the first organism and the second organism are cultured separately. In such embodiments, the first compound is isolated from a culture of cells of the first organism and then provided to a culture of cells of the second organism that converts the first compound into a second compound.

In some embodiments, a synthetic cellular consortium is provided for the production of compounds. In some embodiments, the synthetic cellular consortium produces structurally complex compounds, including terpenoids. As used herein, a terpenoid, also referred to as an isoprenoid, is an organic chemical derived from a five-carbon isoprene unit. Several non-limiting examples of terpenoids, classified based on the number of isoprene units that they contain, include: hemiterpenoids (1 isoprene unit), monoterpenoids (2 isoprene units), sesquiterpenoids (3 isoprene units), diterpenoids (4 isoprene units), sesterterpenoids (5 isoprene units), triterpenoids (6 isoprene units), tetraterpenoids (8 isoprene units), and polyterpenoids with a larger number of isoprene units. In some embodiments, the terpenoid that is produced is taxadiene or a taxadien-5a-ol. In some embodiments, the terpenoid that is produced is an oxygenated taxane, such as taxadien-5a-ol, taxadien-5a-ol-10b-ol or taxadiene-5a-acetate-10b-ol, or an acetylated taxane. In other embodiments the terpenoid that is produced is Citronellol, Cubebol, Nootkatone, Ferruginol, Cineol, Limonene, Eleutherobin, Sarcodictyin, Pseudopterosins, Ginkgolides, Stevioside, Rebaudioside A, sclareol, labdenediol, levopimaradiene, sandracopimaradiene or isopemaradiene. In some embodiments, the compounds produced are monoacetylated deoxygenated taxanes. In other embodiments of the invention, the compounds produced by the synthetic cellular consortium include, without limitation, polyketides, alkaloids, flavonoids, short chain dicarboxylic acids, and recombinant proteins.

In some embodiments, a synthetic cellular consortium is provided for the production of aromatic compounds or aromatic-derived compounds. As used herein, an aromatic compound is an organic chemical with a conjugated ring structure of unsaturated bonds. Several non-limiting examples of aromatic compounds include 3-aminobenzoate, 4-aminobenzoate, p-hydroxybenzoate, shikimate, protocatechuic acid, catechol, vanillin, gallic acid, anthranilate, tyrosine, phenylalanine, and tryptophan. As used herein, an aromatic-derived compound is a compound for which the biosynthesis uses an aromatic intermediate. A non-limiting example of aromatic-derived compound is muconic acid. In some embodiments, the aromatic compounds or aromatic-derived compounds are produced using the shikimate biosynthetic pathway or portion thereof. In some embodiments, the aromatic compounds or aromatic-derived compounds are produced using the intermediate DHS.

As used herein “cis, cis-muconic acid” and “muconic acid” are used interchangeably and refer to cis, cis-muconic acid.

As used herein, an “intermediate” or “first compound” refers to any compound produced by the biosynthetic pathway that is not the final, intended product. Also used herein, a “second compound” refers to any compound produced by the biosynthetic pathway including the final, intended product.

Synthesis of terpenoids, such as taxadiene, taxadien-5a-ol and oxygenated or acetylated taxanes, such as monoacetylated deoxygenated taxanes; aromatic compounds, such as 3-aminobenzoate and p-hydroxybenzoate; and aromatic-derived compounds, such as muconic acid; is demonstrated herein by use of a synthetic cellular consortium. The use of a synthetic cellular consortium to synthesize complex molecules, like terpenoids, aromatics and aromatic-derived compounds, short chain dicarboxylic acids, and recombinant proteins, can dramatically reduce the cost of production of such compounds. Additionally, a synthetic cellular consortia utilizes cheap, abundant and renewable feedstocks (such as sugars and other carbohydrates) and can be used for the synthesis of numerous compounds that may exhibit far superior properties than the original compound. Additionally, the surprising success of segmenting a long biosynthetic pathway into two distinct cells, allows for independent optimization of each portion of the pathway to increase production potential.

Described herein are methods for synthesizing compounds in a modular manner by producing an intermediate compound by a first organism that is then further modified by one or more additional organisms. In some embodiments the first organism and the second organism are co-cultured within a shared environment. In such embodiments, the intermediate compound is released into the culture environment by the first organism and can be internalized and further processed by the second organism. In other embodiments, the first organism and the second organism are cultured in separate environments. In such embodiments, the intermediate compound is isolated from the culture of cells of the first organism. Then the intermediate compound is provided to the culture of cells of the second organism, which can internalized and further process the compound.

In some embodiments, methods are provided for the synthesis of complex isoprenoids using a cellular consortium. In such embodiments, the first organisms are genetically engineered to amplify the metabolic flux to the synthesis of isopentenyl pyrophosphate (IPP) and dimethylallyl pyrophosphate (DMAPP), key intermediates for the production of isoprenoid compounds, which can be further converted into geranyl geranyl diphosphate (GGPP), then taxadiene. Additionally described herein are methods that enhance functionalization of taxadiene in the second organism. Specifically, these particular organisms are genetically engineered to allow sequential hydroxylation reactions of the precursor compound to produce paclitaxel (Taxol), ginkolides, geraniol, farnesol, geranylgeraniol, linalool, isoprene, monoterpenoids such as menthol, carotenoids such as lycopene, polyisoprenoids such as polyisoprene or natural rubber, diterpenoids such as eleutherobin, sesquiterpenoids such as artemisinin, monoacetylated deoxygenated taxanes, and other oxygenated isoprenoids, such as ferruginol or nootkatone.

In embodiments for the synthesis of oxygenated isoprenoids, such as ferruginol, the first organisms are genetically engineered to produce miltiradiene, an intermediate for the production of ferruginol. in embodiments in which the first organism produces miltiradiene, any second organism that is able to convert miltiradiene into a second compound is compatible for use in the invention, such as a second organism that is engineered to oxygenate miltiradiene to ferruginol.

In embodiments for the synthesis of oxygenated isoprenoids, such as nootkatone, the first organisms are genetically engineered to produce valencene, an intermediate for the production of nootkatone. in embodiments in which the first organism produces valencene, any second organism that is able to convert valencene into a second compound is compatible for use in the invention, such as a second organism that is engineered to oxygenate valencene to nootkatone.

In other embodiments, methods are provided for the synthesis of aromatic compounds or aromatic-derived compounds using a synthetic cellular consortium. In such embodiments, the first organism is responsible for the production of DHS, a key intermediate for the production of aromatic or aromatic-derived compounds. In some embodiments, the first organism is genetically engineered to increase production of enzymes involved in the shikimate pathway. In some embodiments, the first organism is genetically engineered to increase production of DHS. Additionally described herein are methods that convert the intermediate into an aromatic or aromatic-derived compound. A benefit of this synthetic cellular consortium system is that the second organism can be varied depending on the desired product. For example, in embodiments in which the first organism produces DHS, any second organism that is able to convert DHS into a second compound is compatible for use in the invention, such as a second organism that is engineered to convert DHS into muconic acid, 3-aminobenzoate, or p-hydroxybenzoate.

In some embodiments, methods are provided for the synthesis of aromatic-derived compounds, such as alkaloids, using a synthetic cellular consortium. In such embodiments, the first organism is responsible for the production of an aromatic amino acid (e.g., tyrosine). In some embodiments, the first organism is genetic engineered to increase production of aromatic amino acids. In embodiments in which the first organism produces an aromatic amino acid, any second organism that is able to convert the aromatic amino acid into a second compound is compatible for use in the invention, such as a second organism that is engineered to convert the aromatic amino acid into a product, such as (S)-reticuline.

Cells that are genetically engineered to recombinantly express one or more genes or enzymes of the terpenoid biosynthetic pathway and methods to use such cells are provided. Cells that are genetically engineered to recombinantly express one or more genes or enzymes of the shikimate biosynthetic pathway and methods to use such cells are also provided. As used herein “genetic engineering” refers to the manipulation an organism's nucleic acid. In some embodiments genetic engineering involves insertion of a gene, deletion of a gene, or modulation of expression of a gene. “Recombinant expression” refers to enhancing or increasing the expression of genes or proteins above levels that would be achieved without such a strategy. Recombinant expression also pertains to expression of a gene or protein in an organism that does not normally express the particular gene or protein.

Embodiments of the invention described herein pertain to segmenting a biosynthetic pathway into more than one cell to produce a final compound. For example, the first organism synthesizes a first compound, or an intermediate of a biosynthetic pathway, which is then further processed by a second organism into a second compound. In some embodiments the second compound is further processed by a third organism into a third compound. In some embodiments of the invention, the first and second organisms are bacteria. In some embodiments of the invention, the first and second organisms are yeast. In some embodiments, the first and second organisms are plant cells. In some embodiments, the first organism is a bacterium and the second organism is a yeast. In some embodiments, the first organism is a yeast and the second organism is a bacterium. In some embodiments, the first organism is a bacterium and the second organism is a plant cell. In some embodiments, the first organism is a yeast and the second organism is a plant cell.

In some embodiments, the biosynthetic pathway that is segmented into at least two modules is a terpenoid synthesis pathway. In some embodiments the first compound is an intermediate of the MEP pathway. In some embodiments the second compound is a terpenoid. In some embodiments, the first compound is an intermediate of the MEP pathway, and the second compound is a monoacetylated deoxygenated taxane. In other embodiments, the first compound is amorphadiene and the second compound is artemisinin. In other embodiments, the first compound is valencene and the second compound is nootkatone. In other embodiments, the first compound is miltiradiene and the second compound is ferruginol. In still other embodiments, the biosynthetic pathway that is segmented into at least two modules is a polyketide synthesis pathway. In these embodiments, the first compound produced by the first organism is an intermediate of the polyketide pathway that is further processed by a second organism to produce a polyketide. In other embodiments, the biosynthetic pathway that is segmented into at least two modules is an alkaloid synthesis pathway. In these embodiments, the first compound produced by the first organism is an intermediate of the alkaloid pathway that is further processed by a second organism to produce an alkaloid. In some embodiments, the first compound is an aromatic amino acid, and the second compound is an alkaloid. In some embodiments, the first compound is an aromatic amino acid, and the second compound is a flavonoid.

In some embodiments, the biosynthetic pathway that is segmented into at least two modules is the shikimate pathway. In some embodiments, the first module is a portion of the shikimate pathway. In some embodiments, the second module is a second synthetic pathway or portion thereof. In some embodiments, the first compound is an intermediate of the shikimate pathway (e.g., DHS, shikimate). In some embodiments, an intermediate of the shikimate pathway is further processed by a second organism to produce an aromatic compound. In some embodiments, the aromatic compound is 3-aminobenzoate or p-hydroxybenzoate. In some embodiments, an intermediate of the shikimate pathway is further processed by a second organism to produce an aromatic-derived compound. In some embodiments, the aromatic-derived compound is muconic acid.

In yet other embodiments, the first compound is a short chain fatty acid, and the second compound is a short chain dicarboxylic acid.

Described herein are methods and compositions for production of terpenoids in a segmented manner by recombinantly expressing genes or proteins participating in steps of the biosynthetic pathway. The first portion of the pathway involves production of isopentyl pyrophosphate (IPP) and dimethylallyl pyrophosphate (DMAPP), which can be achieved by two different metabolic pathways: the mevalonic acid (MVA) pathway and the MEP (2-C-methyl-D-erythritol 4-phosphate) pathway, also called the MEP/DOXP (2-C-methyl-D-erythritol 4-phosphate/1-deoxy-D-xylulose 5-phosphate) pathway, the non-mevalonate pathway or the mevalonic acid-independent pathway. Both IPP and DMAPP must be cyclized into an intermediate compound, taxadiene. These steps are achieved by recombinant gene expression of a GGPPS enzyme that linearly couples the precursor to GGPP and a terpenoid synthase enzyme (also referred to as terpene cyclase) the cyclizes the molecule. The GGPPS enzyme belongs to a prenyltransferase type family of enzymes that can accept multiple substrates, including but not limited to DMAPP, farnesyl diphosphate (FPP), geranyl diphosphate (GPP), and farnesyl geranyl diphosphate (FGPP) to produce a variety of different terpenoids. In some embodiments, the terpenoid synthase enzyme is a diterpenoid synthase enzyme. Several non-limiting examples of terpenoid synthase enzymes include taxadiene synthase, casbene synthase, levopimaradiene synthase, abietadiene synthase, isopimaradiene synthase, ent-copalyl diphosphate synthase, syn-stemar-13-ene synthase, syn-stemod-13(17)-ene synthase, syn-pimara-7,15-diene synthase, ent-sandaracopimaradiene synthase, ent-cassa-12,15-diene synthase, ent-pimara-8(14), 15-diene synthase, ent-kaur-15-ene synthase, ent-kaur-16-ene synthase, aphidicolan-16β-ol synthase, phyllocladan-16α-ol synthase, fusicocca-2,10(14)-diene synthase, and terpentetriene cyclase. In some embodiments the terpenoid synthase and the GGPPS enzyme are expressed as a single polypeptide that retains the activities of each of the two proteins.

The terpenoid pathway intermediate taxadiene is subjected to sequential hydroxylation reactions to produce functionalized oxygenated taxanes. In some embodiments, this involves recombinant expression of components of a plant cytochrome P450. In some embodiments the plant cytochrome P450 is a taxadiene 5α hydroxylase and its reductase. In other embodiments the hydroxylation reactions involve recombinant expression of taxane-10-beta-hydroxylase. In other embodiments the hydroxylation reactions involve recombinant expression of taxa-4(20), 11(12)-dien-5α-ol O-acetyltransferase.

Embodiments of the invention described herein relate to production of terpenoids by segmenting the biosynthetic pathway into two or more cells. In some embodiments genes or proteins of the MEP pathway and/or the GGPPS and TS enzymes are expressed within a single organism, referred to as the “first organism,” such that the first organism produces an intermediate of the pathway, referred to as the “first compound”. In some embodiments the first compound produced by the first organism is taxadiene. In some embodiments. In some embodiments, the plant cytochrome P450 is expressed in the second organism such that the second organism produces a second compound. In some embodiments the second compound is an oxygenated taxane.

In some embodiments, the first organism further expresses a taxadien-5α-ol acetyltransferase. In some embodiments, the second organism expresses a taxane 10β-hydroxylase.

In some embodiments, the first organism expresses the diterpene synthases KSL and CPS and produces the first compound miltiradiene. In some embodiments, the first organism expresses the sesquiterpene synthase VALC and produces the first compound valencene.

Described herein are methods and compositions for production of aromatic compounds or aromatic-derived compounds in a segmented manner by recombinantly expressing genes or proteins participating in steps of the biosynthetic pathway. The first portion of the pathway involves production of an intermediate of the shikimate pathway, for example DHS. Production of DHS and components of the shikimate pathway can be enhanced by recombinantly expressing global transcription machinery genes, including engineered global transcription machinery genes. In some embodiments, the production of DHS and components of the shikimate pathway are enhanced by recombinantly expressing an RNA polymerase with one or more mutations. In some embodiments, the global transcription machinery gene is rpoA encoding an a subunit of RNA polymerase. Mutations in rpoA that enhance production of desired compounds will be evident to one of skill in the art and can be found, for example in Santos et al. PNAS 2012, 109(34): 13538-43. In some embodiments, the production of DHS is enhanced by deleting or mutating one or more genes encoding a shikimate dehydrogenase. Production of the aromatic-derived compound, muconic acid, from the intermediate DHS is achieved by recombinant expression of a DHS dehydratase (EC 4.2.1.118) to convert DHS to protocatechuic acid (PCA); a PCA decarboxylase (EC 4.1.1.63) to convert PCA to catechol; and a catechol 1,2-dioxygenase (EC 1.13.11.1) to convert catechol into muconic acid. In some embodiments, a first organism is engineered to produce DHS and a second organism is engineered to recombinantly express a DHS dehydratase, a PCA decarboxylase, and a catechol 1,2-dioxygenase in order to convert DHS to muconic acid. Production of p-hydroxybenzoate (PHB) from the intermediate DHS is achieved by recombinant expression of a shikimate dehydrogenase (EC 1.1.1.282 or EC 1.1.1.25) to convert DHS to shikimate; a shikimate kinase (EC 2.7.1.71) to convert shikimate to shikimate-3-phosphate (S3P); a 5-enolpyruvyl shikimate 3-phosphate synthase (EC 2.5.1.19) to convert S3P to enolpyruvyl shikimate 3-phosphate (EPSP); a chorismate synthase (EC 4.2.3.5) to convert EPSP to chorismate; and a chorismate pyruvate lyase (EC 4.1.3.40) to convert chorismate to PHB. In some embodiments, a first organism is engineered to produce DHS and a second organism is engineered to recombinantly express a shikimate dehydrogenase, shikimate kinase, 5-enolpyruvyl shikimate 3-phosphate synthase, chorismate synthase, and a chorismate pyruvate lyase in order to convert DHS to PHB. Production of 3-aminobenzoate is achieved by recombinant expression of an amino transferase to convert DHS to 3-aminobenzoate. In some embodiments, a first organism is engineered to produce DHS and a second organism is engineered to recombinantly express an amino transferase in order to convert DHS to 3-aminobenzoate. In any of the methods described herein, it may be advantageous to further recombinantly express a transporter in the second organism to improve uptake of the intermediate. In some embodiments, the transporter is the ShiA permease that can import DHS.

In some embodiments of the invention, there also is a mutualistic relationship between the first and second organisms of the consortium. For example, the first organism utilizes a nutritional source provided in the liquid culture medium and a byproduct produced by the degradation of the first nutritional source serves a nutritional source for the second organism. In some embodiments, the nutritional source for the first organism provided in the liquid culture medium is a carbon source. In some embodiments the carbon source for the first organism is xylose. In some embodiments the byproduct produced by the first organism may be a carbon source for the second organism. In some embodiments, the carbon source for the second organism is acetate.

In some embodiments, the first and second organisms of the consortium utilize different nutritional sources provided in the liquid culture medium. In some embodiments, the nutritional source for the first organism provided in the liquid culture medium is a carbon source that is not utilized by the second organism. In some embodiments the carbon source for the first organism is xylose. In some embodiments, the nutritional source for the second organism provided in the liquid culture medium is a carbon source that is not utilized by the first organism. In some embodiments the carbon source for the second organism is glucose. In some embodiments, the first organism may be genetically engineered to not utilize the carbon source that used by the second organism. In some embodiments, a glucose uptake system is mutated or deleted in the first organism. In some embodiments, the second organism may be genetically engineered to not utilize the carbon source that used by the first organism. In some embodiments, a xylose utilization system is mutated or deleted in the second organism.

In some embodiments, the first and second organisms are cultured independently. In some embodiments the first organism produces an intermediate in its own culture environment. The intermediate, or first compound, is then isolated or purified from the culture of the first organism and added to the culture of the second organism where the first compound is converted into a second compound.

Aspects of the invention relate to expression of recombinant genes in a first organism. In some embodiments, the invention relates to recombinant expression of genes in two or more organisms. The invention encompasses any type of cell that recombinantly expresses genes associated with the invention, including prokaryotic and eukaryotic cells. In some embodiments the cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an Escherichia coli (E. coli) cell, or a Gram-positive cell such as a species of Bacillus. In other embodiments, the cell is a fungal cell such as a yeast cell, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp., and industrial polyploid yeast strains. Preferably the yeast strain is a S. cerevisiae strain or a Yarrowia spp. strain. Other examples of fungi include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In other embodiments, the cell is an algal cell, or a plant cell, e.g. Taxus spp. In some embodiments, the plant cell is a Taxus cuspidata cell. It should be appreciated that some cells compatible with the invention may express an endogenous copy of one or more of the genes associated with the invention as well as a recombinant copy. In some embodiments, if a cell has an endogenous copy of one or more of the genes associated with the invention then the methods will not necessarily require adding a recombinant copy of the gene(s) that are endogenously expressed. In some embodiments the cell may endogenously express one or more enzymes from the pathways described herein and may recombinantly express one or more other enzymes from the pathways described herein for efficient production of a desired compound (e.g., terpenoid, taxanes, aromatic, aromatic-derived compound).

Aspects of the invention relate to controlling the expression of genes and proteins of the upstream and downstream pathways for production of a desired compound such as a terpenoid (e.g., taxadiene, oxygenated taxanes), aromatic compound (e.g. PHB, 3-aminobenzoate), an aromatic-derived compound (e.g., muconic acid, alkaloids, flavonoids), short chain dicarboyxlic acids, or recombinant proteins. Recombinant expression refers to enhancing or increasing the expression of genes or proteins above levels that would be achieved without such a strategy. Recombinant expression also pertains to expression of a gene or protein in an organism that does not ordinarily express the particular gene or protein. It should be appreciated that any gene and/or protein within the MEP pathway is encompassed by methods and compositions described herein. In some embodiments, a gene within the MEP pathway is one of the following: dxs, ispC, ispD, ispE, ispF, ispG, ispH, idi, ispA or ispB. Expression of genes within the MEP pathway can be regulated in a modular method. One or more genes and/or proteins for the production of oxygenated isoprenoids (e.g., ferruginol and nootkatone) are encompassed by the methods and compositions described herein. In some embodiments, genes involved in the production of ferruginol are ksl and cps. In some embodiments, a gene involved in the production of nootkatone is valc. It should also be appreciated that any gene and/or protein within the shikimate pathway is encompassed by methods and compositions described herein. In some embodiments, a gene within the shikimate pathway may be a gene involved in the production of DHS, such as aroA, aroB, or aroC. In some embodiments, a gene within the shikimate pathway may be a gene involved in the production of PHB, such as aroE, aroL, aroA, aroC, or ubiC. Furthermore, one or more genes and/or proteins for the production of muconic acid are encompassed by methods and compositions described herein. In some embodiments, a gene involved in the production of muconic acid may be aroZ, aroY, or catA. Additionally, one or more genes and/or proteins for the production of 3-aminobenzaote are also encompassed by methods and compositions described herein. In some embodiments, a gene involved in the production of 3-aminobenzoate is pctV.

As used herein, regulation by a modular method refers to regulation of multiple genes together. For example, in some embodiments, multiple genes within of a pathway are recombinantly expressed on a contiguous region of DNA, such as an operon. It should be appreciated that a cell that expresses such a module can also express one or more other genes within the same pathway or a different pathway either recombinantly or endogenously.

A non-limiting example of a module of genes within the MEP pathway is a module containing the genes dxs, idi, ispD and ispF, and referred to herein as dxs-idi-ispDF. It should be appreciated that modules of genes within the MEP pathway, consistent with aspects of the invention, can contain any of the genes within the MEP pathway, in any order. A non-limiting example of a module of genes within the shikimate pathway for the production of PHB is a module containing the genes aroE, aroL, aroA, aroC, and ubiC, referred to herein as ELACU. A non-limiting example of a module of genes for the production of muconic acid is a module containing the genes aroZ, aroY, and catA.

Expression of genes and proteins within any of the pathways described herein can be regulated in order to optimize production of a desired compound. For example, the synthetic downstream terpenoid synthesis pathway involves recombinant expression of a terpenoid synthase enzyme and a GGPPS enzyme. Any terpenoid synthase enzyme, as discussed above, can be expressed with GGPPS depending on the downstream product to be produced. For example, taxadiene synthase is used for the production of taxadiene. Recombinant expression of the taxadiene synthase enzyme and the GGPPS enzyme can be regulated independently or together. In some embodiments the two enzymes are regulated together in a modular fashion

Expression of genes and proteins within the functionalization/oxygenation pathways can also be regulated to optimize terpenoid production. This functionalization/oxygenation pathway involves recombinant expression of components of the taxadiene 5α hydroxylase and its reductase. Recombinant expression of the taxadiene 5α hydroxylase and reductase can be regulated independently or together. In some embodiments, the two enzymes can be regulated together in a modular fashion. In some embodiments, expression of the genes and proteins within the functionalization/oxygenation pathway may be endogenous.

Manipulation of the expression of genes and/or proteins, including modules can be achieved through methods known to one of ordinary skill in the art. For example, expression of the genes or operons can be regulated through selection of promoters, such as constitutively active or inducible promoters. Several non-limiting examples of constitutively active promoters include T7, sigma 70, the translation elongation factor 1α promoter (TEF), the glyceraldehyde-3-phophate dehydrogenase promoter (GPD), the glyceraldehyde-3-phophate dehydrogenase promoter including upstream activation sequence elements (UAS-GPD), and the acyl-coenzyme A synthetase (ACS) promoter. Several non-limiting examples of inducible promoters include a lactose or IPTG-inducible promoter, an L-arabinose-inducible promoter, a L-rhamnose-inducible promoter, tetracycline-inducible promoter, tryptophan-inducible promoter, and a phosphate-inducible promoter.

It should be appreciated that the genes associated with the invention can be obtained from a variety of sources. In some embodiments, the genes within the MEP pathway are bacterial genes such as Escherichia coli genes. In some embodiments, the gene encoding for GGPPS is a plant gene. For example, the gene encoding for GGPPS can be from a species of Taxus such as Taxus canadensis (T. canadensis). In some embodiments, the gene encoding for taxadiene synthase is a plant gene. For example, the gene encoding for taxadiene synthase can be from a species of Taxus such as Taxus brevifolia (T. brevifolia). In some embodiments, the genes encoding for the plant cytochrome P450 components taxadiene 5 hydroxylase and its reductase are plant genes. For example, the gene encoding for taxadiene 5 hydroxylase and its reductase can be from a species of Taxus such as Taxus cuspidata. Representative GenBank Accession numbers for T. canadensis GGPPS, T. brevifolia taxadiene synthase, and T. cuspidata taxadiene 5 hydroxylase and its reductase are provided by AF081514, U48796, AY289209, and AY571340 the sequences of which are incorporated by reference herein in their entireties. In some embodiments, the genes within the shikimate pathway are bacterial genes. In some embodiments, the aroZ and/or aroY genes are Klebsiella pneumoniae genes. In some embodiments, the catA gene is an Acinetobacter calcoaceticus gene. In some embodiments, the aroE, aroL, aroA, aroC, and ubiC genes are Escherichia coli genes. In some embodiments, the pctV gene is a Streptomyces pactum gene.

In some embodiments, the gene encoding the taxadien-5-αol acetyl-transferase (TAT) is from a species of Taxus, such as Taxus cuspidata. In some embodiments, the gene encoding the taxadien-5-αol acetyl-transferase (TAT) is provided by SEQ ID NO: 96. In some embodiments, the gene encoding the taxane 10 β-hydroxylase (10βCYP) is from a species of Taxus, such as Taxus cuspidata. In some embodiments, the gene encoding the taxane 10 β-hydroxylase (10βCYP) is provided by SEQ ID NO: 97. In some embodiments, the gene encoding KSL is from a species of Salvia, such as Salvia miltiorrhiza. In some embodiments, the gene encoding KSL is provided by SEQ ID NO: 98. In some embodiments, the gene encoding CPS is from a species of Salvia, such as Salvia miltiorrhiza.

In some embodiments, the gene encoding CPS is provided by SEQ ID NO: 99. In some embodiments, the gene encoding SmCYP is from a species of Salvia, such as Salvia miltiorrhiza. In some embodiments, the gene encoding SmCYP is provided by SEQ ID NO: 100. In some embodiments, the gene encoding SmCYP is from a species of Salvia, such as Salvia miltiorrhiza. In some embodiments, the gene encoding SmCPR is provided by SEQ ID NO: 101. In some embodiments, the gene encoding ValC is from a species of Callitropsis, such as Callitropsis nootkatensis. In some embodiments, the gene encoding ValC is provided by SEQ ID NO: 102. In some embodiments, the gene encoding HmCYP is from a species of Hyoscyamus, such as Hyoscyamus muticus. In some embodiments, the gene encoding HmCYP is provided by SEQ ID NO: 103. In some embodiments, the gene encoding AtCPR is from a species of Arabidopsis, such as Arabidopsis thaliana. In some embodiments, the gene encoding AtCPR is provided by SEQ ID NO: 104.

As one of ordinary skill in the art would be aware, homologous genes for use in methods associated with the invention can be obtained from other species and can be identified by homology searches, for example through a protein BLAST search, available at the National Center for Biotechnology Information (NCBI) internet site (www.ncbi.nlm.nih.gov). Genes and/or operons associated with the invention can be cloned, for example by PCR amplification and/or restriction digestion, from DNA from any source of DNA which contains the given gene. In some embodiments, a gene and/or operon associated with the invention is synthetic. Any means of obtaining a gene and/or operon associated with the invention is compatible with the instant invention.

In some embodiments, further optimization of terpenoid production is achieved by modifying a gene before it is recombinantly expressed in a cell. In some embodiments, the GGPPS enzyme has one or more of the follow mutations: A162V, G140C, L182M, F218Y, D160G, C184S, K367R, A151T, M185I, D264Y, E368D, C184R, L331I, G262V, R365S, A114D, S239C, G295D, I276V, K343N, P183S, I172T, D267G, I149V, T234I, E153D and T259A. In some embodiments, the GGPPS enzyme has a mutation in residue S239 and/or residue G295. In certain embodiments, the GGPPS enzyme has the mutation S239C and/or G295D.

In some embodiments, modification of a gene before it is recombinantly expressed in a cell involves codon optimization for expression in a bacterial, yeast, or plant cell. Codon usages for a variety of organisms can be accessed in the Codon Usage Database (www.kazusa.or.jp/codon/). Codon optimization, including identification of optimal codons for a variety of organisms, and methods for achieving codon optimization, are familiar to one of ordinary skill in the art, and can be achieved using standard methods.

In some embodiments, modifying a gene before it is recombinantly expressed in a cell involves making one or more mutations in the gene before it is recombinantly expressed in a cell. For example, a mutation can involve a substitution or deletion of a single nucleotide or multiple nucleotides. In some embodiments, a mutation of one or more nucleotides in a gene will result in a mutation in the protein produced from the gene, such as a substitution or deletion of one or more amino acids.

In some embodiments “rational design” is involved in constructing specific mutations in proteins such as enzymes. As used herein, “rational design” refers to incorporating knowledge of the enzyme, or related enzymes, such as its three dimensional structure, its active site(s), its substrate(s) and/or the interaction between the enzyme and substrate, into the design of the specific mutation. Based on a rational design approach, mutations can be created in an enzyme which can then be screened for increased production of a terpenoid relative to control levels. In some embodiments, mutations can be rationally designed based on homology modeling. As used herein, “homology modeling” refers to the process of constructing an atomic resolution model of one protein from its amino acid sequence and a three-dimensional structure of a related homologous protein.

In some embodiments, random mutations can be made in a gene, such as a gene encoding for an enzyme, and these mutations can be screened for increased production of a terpenoid relative to control levels. For example, screening for mutations in components of the MEP pathway, the shikimate pathway, short chain fatty acid oxidation pathways, or components of other pathways, that lead to enhanced production of a desired compound may be conducted through a random mutagenesis screen, or through screening of known mutations. In some embodiments, shotgun cloning of genomic fragments could be used to identify genomic regions that lead to an increase in production of a desired compound, through screening cells or organisms that have these fragments for increased production of the compound. In some cases one or more mutations may be combined in the same cell or organism.

In some embodiments, production of a desired compound (e.g., terpenoid, aromatic or aromatic-derived compound, alkaloids, flavonoids, short chain dicarboxylic acids, recombinant proteins) in a cell can be increased through manipulation of enzymes that act in the same pathway as the enzymes associated with the invention. For example, in some embodiments it may be advantageous to increase expression of an enzyme or other factor that acts upstream of a target enzyme such as an enzyme associated with the invention. This could be achieved by over-expressing the upstream factor using any standard method.

Optimization of protein expression can also be achieved through selection of appropriate promoters and ribosome binding sites. In some embodiments, this may include the selection of high-copy number plasmids, or low or medium-copy number plasmids. The step of transcription termination can also be targeted for regulation of gene expression, through the introduction or elimination of structures such as stem-loops.

Further aspects of the invention relate to screening for bacterial cells or strains that exhibit optimized production of a desired compound (e.g., terpenoid, aromatic or aromatic-derived compound, alkaloid, flavonoid, short chain dicarboxylic acid, recombinant protein). As described above, methods associated with the invention involve generating cells that recombinantly express one or more genes of a synthetic pathway. Production of a desired compound for culturing such cells can be measured and compared to another cell. The cell can be further modified by increasing or decreasing expression of one or more genes or recombinantly expressing one or more additional genes. Production of a desired compound for culturing such cells can be measured again, leading to the identification of an improved cell.

In some embodiments, methods associated with the invention involve generating cells that overexpress one or more genes in the MEP pathway. Terpenoid production from culturing of such cells can be measured and compared to a control cell wherein a cell that exhibits a higher amount of a terpenoid production relative to a control cell is selected as a first improved cell. The cell can be further modified by recombinant expression of a terpenoid synthase enzyme and a GGPPS enzyme. The level of expression of one or more of the components of the non-mevalonate (MEP) pathway, the terpenoid synthase enzyme, the GGPPS enzyme, the 5 taxadiene hydroxylase and/or its reductase in the cell can then be manipulated and terpenoid production can be measured again, leading to selection of a second improved cell that produces greater amounts of a terpenoid than the first improved cell. In some embodiments, the terpenoid synthase enzyme is a taxadiene synthase enzyme. Similarly, methods associated with the invention involve generating cells that recombinantly express one or more genes for the production of an aromatic or aromatic-derived compound. In such embodiments, production of an aromatic or aromatic-derived compound by the cell can be measured. The cell can be further engineered to improve production of the compound.

Some aspects of the invention pertain to optimizing growth or metabolism of cells of the consortium as a method to optimize production of the desired compound. In some embodiments, optimizing growth or metabolism of cells requires increasing the availability of a nutrient in the culture medium. In some embodiments, the first organism is genetically engineered to increase production of a byproduct that can be used as a carbon source by the second organism.

Further aspects of the invention relate to chimeric P450 enzymes. Functional expression of plant cytochrome P450 has been considered challenging due to the inherent limitations of bacterial platforms, such as the absence of electron transfer machinery, cytochrome P450 reductases, and translational incompatibility of the membrane signal modules of P450 enzymes due to the lack of an endoplasmic reticulum.

In some embodiments, the taxadiene-5α-hydroxylase associated with methods of the invention is optimized through N-terminal transmembrane engineering and/or the generation of chimeric enzymes through translational fusion with a CPR redox partner, as has been described in depth (see US 2011/0189717).

As used herein, the terms “protein” and “polypeptide” are used interchangeably and thus the term polypeptide may be used to refer to a full-length polypeptide and may also be used to refer to a fragment of a full-length polypeptide. As used herein with respect to polypeptides, proteins, or fragments thereof, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may be, but need not be, substantially pure. The term “substantially pure” means that the proteins or polypeptides are essentially free of other substances with which they may be found in production, nature, or in vivo systems to an extent practical and appropriate for their intended use. Substantially pure polypeptides may be obtained naturally or produced using methods described herein and may be purified with techniques well known in the art. Because an isolated protein may be admixed with other components in a preparation, the protein may comprise only a small percentage by weight of the preparation. The protein is nonetheless isolated in that it has been separated from the substances with which it may be associated in living systems, i.e. isolated from other proteins.

The invention also encompasses nucleic acids that encode for any of the polypeptides described herein, libraries that contain any of the nucleic acids and/or polypeptides described herein, and compositions that contain any of the nucleic acids and/or polypeptides described herein.

In some embodiments, one or more genes or modules of the invention including the genes of the MEP pathway, GGPPS, terpenoid synthase, components of the P450 cytochrome, e.g., nucleic acid encoding taxadiene 5α hydroxylase and NADPH-cytochrome P450 reductase, genes of the shikimate pathway, and/or any genes involved in the production of aromatic or aromatic-derived compounds, alkaloids, flavonoids, short chain dicarboxylic acids, recombinant proteins may be integrated into the genome of an organism. In some embodiments, the genes may be integrated at a specific site within the genome, such as at the YPRCΔ15 locus.

In some embodiments, one or more of the genes associated with the invention is expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA, although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the enzymes of the claimed invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. Cells are genetically engineered by the introduction into the cells of heterologous DNA (RNA). That heterologous DNA (RNA) is placed under operable control of transcriptional elements to permit the expression of the heterologous DNA in the host cell. Heterologous expression of genes associated with the invention, for production of a terpenoid, such as taxadiene, is demonstrated in the Examples section using E. coli. The novel method for producing terpenoids can also be expressed in other bacterial cells, fungi (including yeast cells), plant cells, etc.

A nucleic acid molecule that encodes an enzyme associated with the invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

In some embodiments one or more genes associated with the invention is expressed recombinantly in a bacterial cell. Bacterial cells according to the invention can be cultured in media of any type (rich or minimal) and any composition. As would be understood by one of ordinary skill in the art, routine optimization would allow for use of a variety of types of media. The selected medium can be supplemented with various additional components. Some non-limiting examples of supplemental components include glucose, xylose, antibiotics, IPTG for gene induction, ATCC Trace Mineral Supplement, and glycolate. Similarly, other aspects of the medium, and growth conditions of the cells of the invention may be optimized through routine experimentation. In some embodiments, the selected medium can be supplemented with lignocellulose or any other complex mixture of carbon sources. For example, pH and temperature are non-limiting examples of factors which can be optimized. In some embodiments, factors such as choice of media, media supplements, and temperature can influence production levels of the desired compound (e.g., terpenoids, such as taxadiene, aromatics or aromatic-derived compounds, alkaloids, flavonoids, short chain dicarboxylic acids, recombinant proteins). In some embodiments the concentration and amount of a supplemental component may be optimized. In some embodiments, how often the media is supplemented with one or more supplemental components, and the amount of time that the media is cultured before harvesting the desired compound, is optimized.

According to aspects of the invention, high titers of a terpenoids such as taxadiene, taxadien-5a-ol or oxygenated taxanes are produced through the recombinant expression of genes associated with the invention, in a synthetic cellular consortium. As used herein “high titer” refers to a titer in the milligrams per liter (mg L⁻¹) scale. The titer produced for a given product will be influenced by multiple factors including choice of media. In some embodiments, the total titer of taxadiene, taxadien-5a-ol or oxygenated taxane is at least 1 mg L⁻¹. In some embodiments, the total titer of taxadiene, taxadien-5a-ol or oxygenated taxane is at least 10 mg L⁻¹. In some embodiments, the total titer of taxadiene, taxadien-5a-ol or oxygenated taxane is at least 250 mg L⁻¹. In some embodiments, the total titer of taxadiene, taxadien-5a-ol or oxygenated taxane is at least 2500 mg L⁻¹. For example, the total titer of taxadiene, taxadien-5a-ol or oxygenated taxane can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900 or more than 900 mg L⁻¹ including any intermediate values. In some embodiments, the total titer of taxadiene, taxadien-5a-ol or oxygenated taxane can be at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, or more than 25.0 g L⁻¹ including any intermediate values. In some embodiments, the total titer of taxadiene, taxadien-5a-ol or oxygenated taxane comprises 20-25000 mg/L, such as 20-1000 mg/L, 50-1000 mg/L, 100-1000 mg/L, 20-5000 mg/L, 50-5000 mg/L, 1000-5000 mg/L, 2000-5000 mg/L, 20-10000 mg/L, 100-10000 mg/L, 1000-10000 mg/L, 2000-10000 mg/L, 20-25000 mg/L, 100-25000 mg/L, 1000-25000 mg/L, 2000-25000 mg/L, or 5000-25000 mg/L. In some embodiments, the taxadiene, taxadien-5a-ol or oxygenated taxane is present in a supernatant of a culture of a synthetic cellular consortium, and can be isolated or purified therefrom.

In some aspects of the invention, high titers of an aromatic-derived compound, such as muconic acid, are produced through the recombinant expression of genes associated with the invention, in a synthetic cellular consortium. For example, the total titer of muconic acid can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900 or more than 900 mg L⁻¹ including any intermediate values. In some embodiments, the total titer of muconic acid comprises 20-2500 mg/L, such as 20-1000 mg/L, 50-1000 mg/L, 100-1000 mg/L, 50-2500 mg/L, 100-2500 mg/L, 1000-2500 mg/L, 2000-2500 mg/L, 20-10000 mg/L, 100-10000 mg/L, 1000-5000 mg/L, 2000-5000 mg/L, 20-5000 mg/L, 100-5000 mg/L, or 500-5000 mg/L. In some embodiments, the muconic acid is present in a supernatant of a culture of a synthetic cellular consortium, and can be isolated or purified therefrom.

In other aspects of the invention, high titers of an aromatic compound, such as PHB or 3-aminobenzoate, are produced through the recombinant expression of genes associated with the invention, in a synthetic cellular consortium. For example, the total titer of PHB or 3-aminobenzoate can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900 or more than 900 mg L⁻¹ including any intermediate values. In some embodiments, the total titer of PHB or 3-aminobenzoate comprises 5-500 mg/L, 3-100 mg/L, 3-50 mg/L, 50-250 mg/L, 5-250 mg/L, 20-2500 mg/L, such as 20-1000 mg/L, 50-1000 mg/L, 100-1000 mg/L, 50-2500 mg/L, 100-2500 mg/L, 1000-2500 mg/L, 2000-2500 mg/L, 20-10000 mg/L, 100-10000 mg/L, 1000-5000 mg/L, 2000-5000 mg/L, 20-5000 mg/L, 100-5000 mg/L, or 500-5000 mg/L. In some embodiments, the PHB or 3-aminobenzoate is present in a supernatant of a culture of a synthetic cellular consortium, and can be isolated or purified therefrom.

In other aspects of the invention, high titers of an alkaloid or flavonoid, are produced through the recombinant expression of genes associated with the invention, in a synthetic cellular consortium. For example, the total titer of the alkaloid or flavonoid can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900 or more than 900 mg L⁻¹ including any intermediate values. In some embodiments, the total titer of the alkaloid or flavonoid comprises 5-500 mg/L, 3-100 mg/L, 3-50 mg/L, 50-250 mg/L, 5-250 mg/L, 20-2500 mg/L, such as 20-1000 mg/L, 50-1000 mg/L, 100-1000 mg/L, 50-2500 mg/L, 100-2500 mg/L, 1000-2500 mg/L, 2000-2500 mg/L, 20-10000 mg/L, 100-10000 mg/L, 1000-5000 mg/L, 2000-5000 mg/L, 20-5000 mg/L, 100-5000 mg/L, 500-5000 mg/L, 2000 mg/L-20 g/L, or 5000 mg/L-50 g/L. In some embodiments, the alkaloid or flavonoid is present in a supernatant of a culture of a synthetic cellular consortium, and can be isolated or purified therefrom.

In yet other aspects of the invention, high titers of a short chain dicarboxylic acid, are produced through the recombinant expression of genes associated with the invention, in a synthetic cellular consortium. For example, the total titer of the short chain dicarboxylic acid can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900 or more than 900 mg L⁻¹ including any intermediate values. In some embodiments, the total titer of the short chain dicarboxylic acid comprises 5-500 mg/L, 3-100 mg/L, 3-50 mg/L, 50-250 mg/L, 5-250 mg/L, 20-2500 mg/L, such as 20-1000 mg/L, 50-1000 mg/L, 100-1000 mg/L, 50-2500 mg/L, 100-2500 mg/L, 1000-2500 mg/L, 2000-2500 mg/L, 20-10000 mg/L, 100-10000 mg/L, 1000-5000 mg/L, 2000-5000 mg/L, 20-5000 mg/L, 100-5000 mg/L, 500-5000 mg/L, 2000 mg/L-20 g/L, or 5000 mg/L-50 g/L. In some embodiments, the short chain dicarboxylic acid is present in a supernatant of a culture of a synthetic cellular consortium, and can be isolated or purified therefrom.

In still other aspects of the invention, high titers of a recombinant protein, are produced through the recombinant expression of genes associated with the invention, in a synthetic cellular consortium. For example, the total titer of the a recombinant protein can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900 or more than 900 mg L⁻¹ including any intermediate values. In some embodiments, the total titer of the a recombinant protein comprises 5-500 mg/L, 3-100 mg/L, 3-50 mg/L, 50-250 mg/L, 5-250 mg/L, 20-2500 mg/L, such as 20-1000 mg/L, 50-1000 mg/L, 100-1000 mg/L, 50-2500 mg/L, 100-2500 mg/L, 1000-2500 mg/L, 2000-2500 mg/L, 20-10000 mg/L, 100-10000 mg/L, 1000-5000 mg/L, 2000-5000 mg/L, 20-5000 mg/L, 100-5000 mg/L, or 500-5000 mg/L. In some embodiments, the a recombinant protein is present in a supernatant of a culture of a synthetic cellular consortium, and can be isolated or purified therefrom.

In some embodiments the synthetic cellular consortium may consist of any combination of bacterial cells, yeast cells and/or plant cells. Each of the cells according to the invention can be cultured in media of any type (rich or minimal) or any composition. As would be understood by one of ordinary skill in the art, a variety of types of media can be used in culturing the synthetic cellular consortium. The selected medium can be supplemented with various additional components. Some non-limiting examples of supplemental components include one or more carbon sources such as glucose, xylose and/or glycerol; antibiotics; and IPTG for gene induction. Similarly, other aspects of the medium, and growth conditions of the cells of the invention may be optimized through routine experimentation. For example, pH and temperature are non-limiting examples of such factors.

The liquid cultures used to maintain the first and second organisms associated with the invention either together or separately can be housed in any of the culture vessels known and used in the art. In some embodiments large scale production in an aerated reaction vessel such as a stirred tank reactor can be used to produce large quantities of a desired compound (e.g., terpenoid, aromatic, aromatic-derived compound, alkaloid, flavonoid, short chain dicarboxylic acid, recombinant protein), that can be recovered from the cell culture. In some embodiments, the desired compound is recovered from the gas phase of the cell culture, for example by adding an organic layer such as dodecane to the cell culture and recovering the compound from the organic layer. In some embodiments, terpenoids can be recovered from the cell culture. In some embodiments, oxygenated taxanes can be recovered from the cell culture. In some embodiments, monoacetylated deoxygenated taxanes can be recovered from the cell culture. In some embodiments, ferruginol can be recovered from the cell culture. In some embodiments, nootkatone can be recovered from the cell culture. In some embodiments, muconic acid can be recovered from the cell culture. In some embodiments, PHB can be recovered from the cell culture. In some embodiments, 3-aminobenzoate can be recovered from the cell culture. In some embodiments, alkaloids can be recovered from the cell culture. In some embodiments, flavonoids can be recovered from the cell culture. In some embodiments, short chain dicarboxylic acids can be recovered from the cell culture. In some embodiments, recombinant proteins can be recovered from the cell culture.

### EXAMPLES

### Example 1

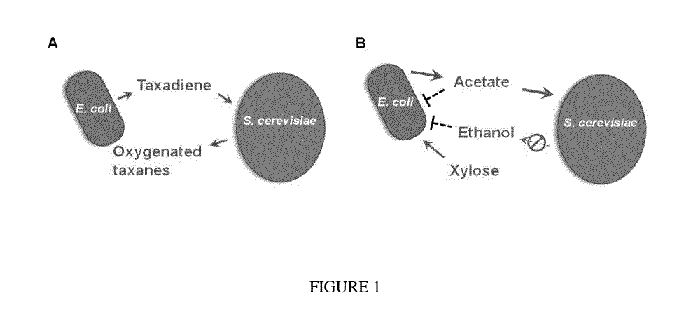

We investigated the properties of a synthetic microbial consortium to produce precursors of the anti-cancer drug paclitaxel by using two model laboratory microbes, E. coli and Saccharomyces cerevisiae. E. coli is a fast growing bacterium that has been previously engineered to produce taxadiene, the scaffold molecule of paclitaxel (Ajikumar et al., 2010; see US 2012/0164678, US 2012/0107893, and US 2011/0189717). S. cerevisiae, having advanced protein expression machinery and abundant intracellular membranes, may be preferable for expressing cytochrome P450s (CYPs), which functionalize taxadiene by catalyzing multiple oxygenation reactions (Guerra-Bubb et al., 2012). Integration of the two species combines rapid production of taxadiene in E. coli with efficient oxygenation of taxadiene by S. cerevisiae (FIG. 1A).

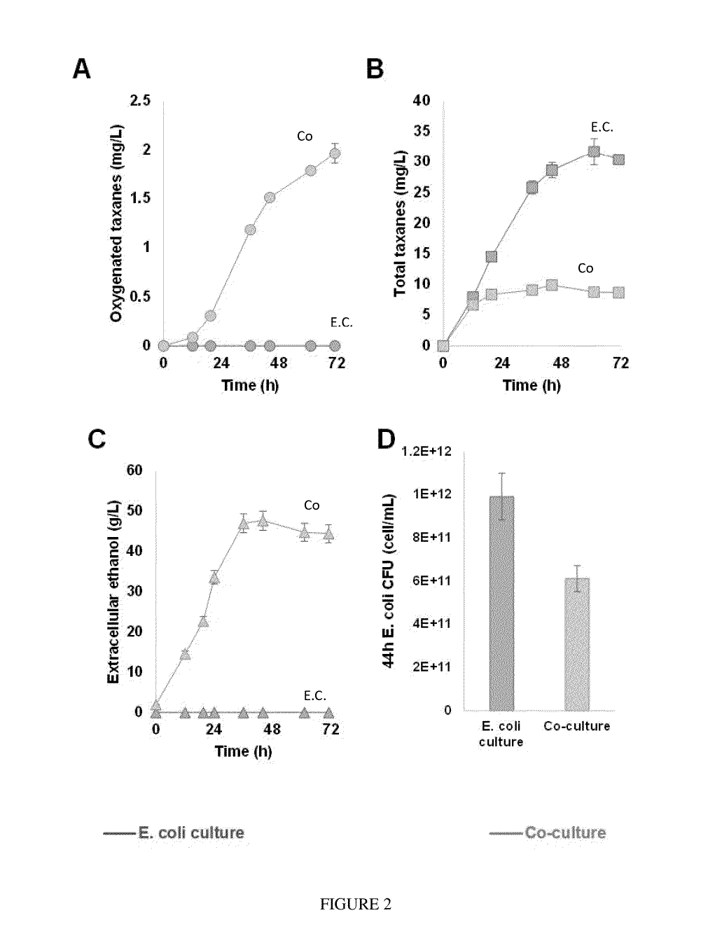

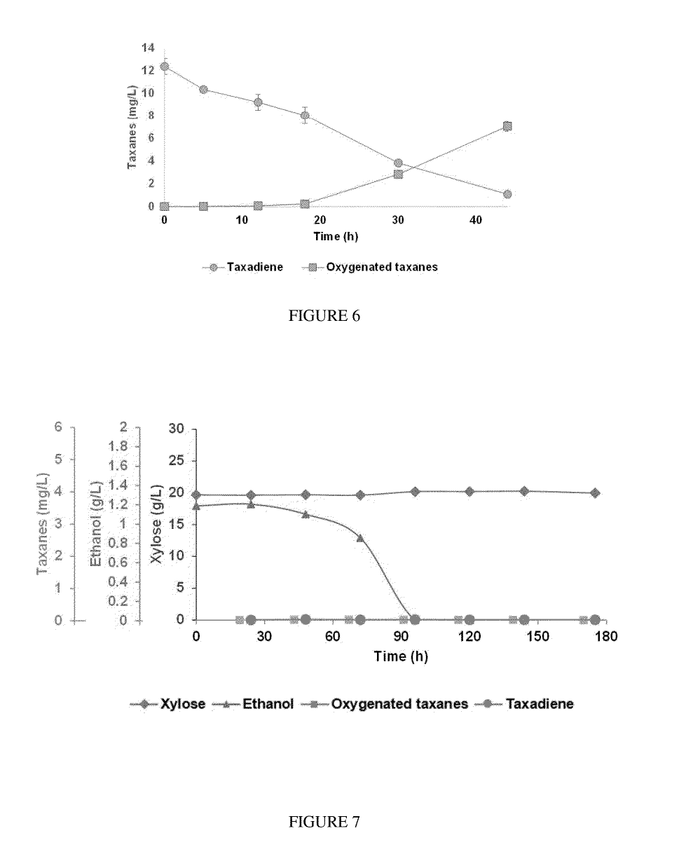

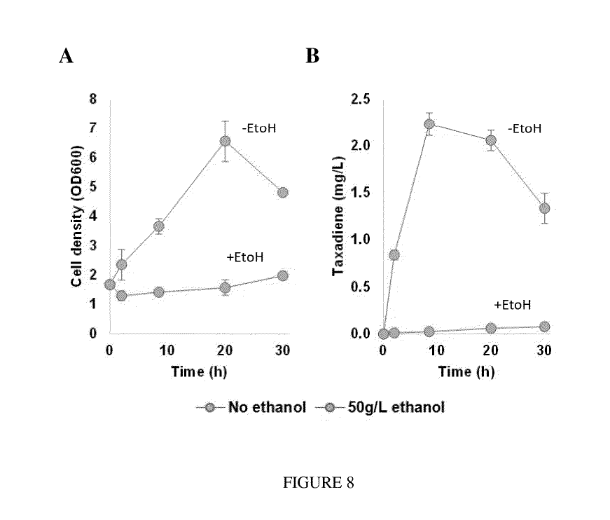

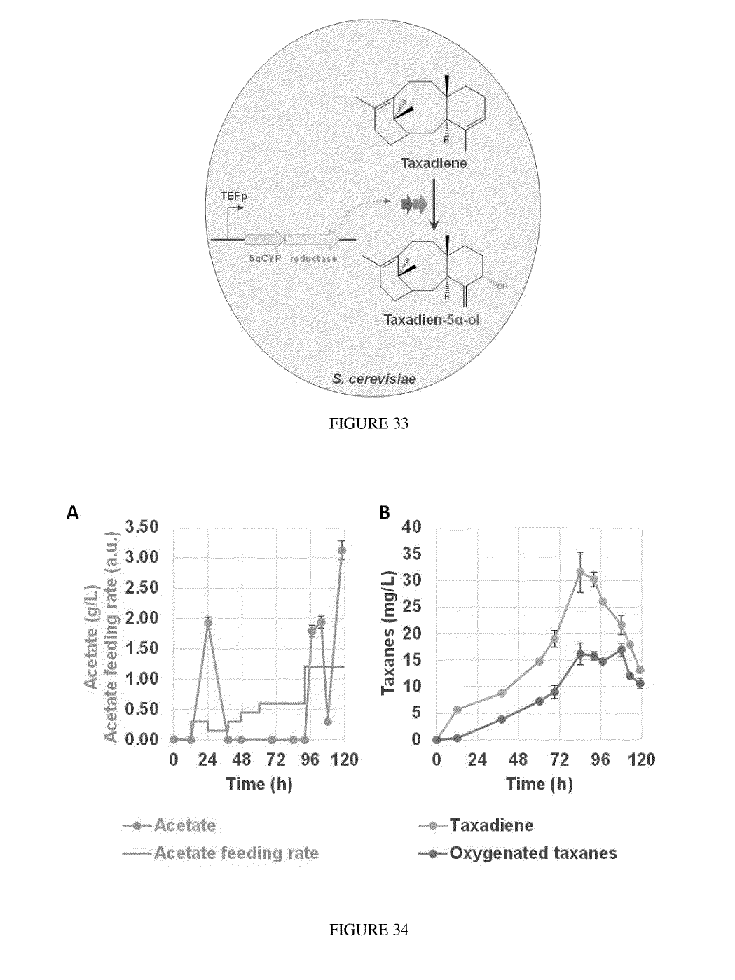

To test this concept, we engineered S. cerevisiae BY4700 to express taxadiene 5α-hydroxylase and its reductase (5αCYP-CPR, FIG. 5, FIG. 33), which catalyze the first oxygenation reaction in the pathway of paclitaxel biosynthesis (Hefner et al., 1996). Taxadiene was found to be efficiently oxygenated by this yeast (named as TaxS1) when it was fed into its culture medium (FIG. 6), confirming that the 5αCYP was functionally expressed in S. cerevisiae. Next, we co-cultured this yeast with a taxadiene-producing E. coli (named as TaxE1) in a fed-batch bioreactor with glucose as carbon and energy source. The mixed culture produced 2 mg/L of oxygenated taxanes in 72 h (FIG. 2A), whereas in control experiments where only E. coli (FIG. 2A) or S. cerevisiae (FIG. 7) was cultured, no oxygenated taxanes were produced. This result supported the hypothesis that taxadiene produced by E. coli can diffuse into S. cerevisiae and be subsequently oxygenated. However, the titer of total taxanes (FIG. 2B) and cell number of E. coli (FIG. 2D) were significantly reduced in the presence of S. cerevisiae. The cause was most likely inhibition of E. coli by accumulated ethanol produced by yeast when grown on glucose (FIG. 2C). As shown in FIG. 2C, ethanol at the highest concentration observed (50 g/L) completely inhibited E. coli cell growth and taxadiene production (FIG. 8). Such inhibition has been frequently observed in natural systems when microbes compete for common resources (Nowak et al., 2006).

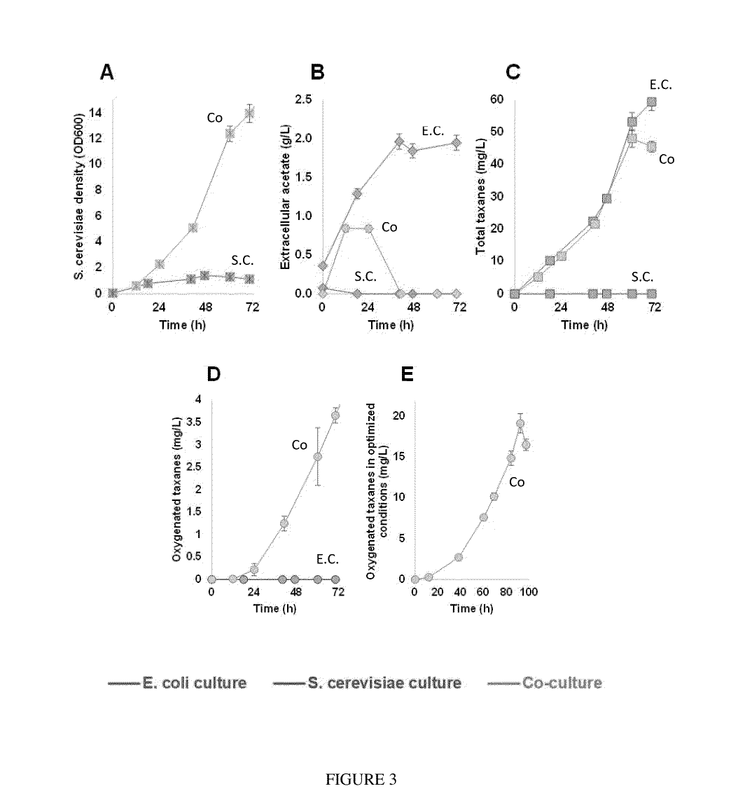

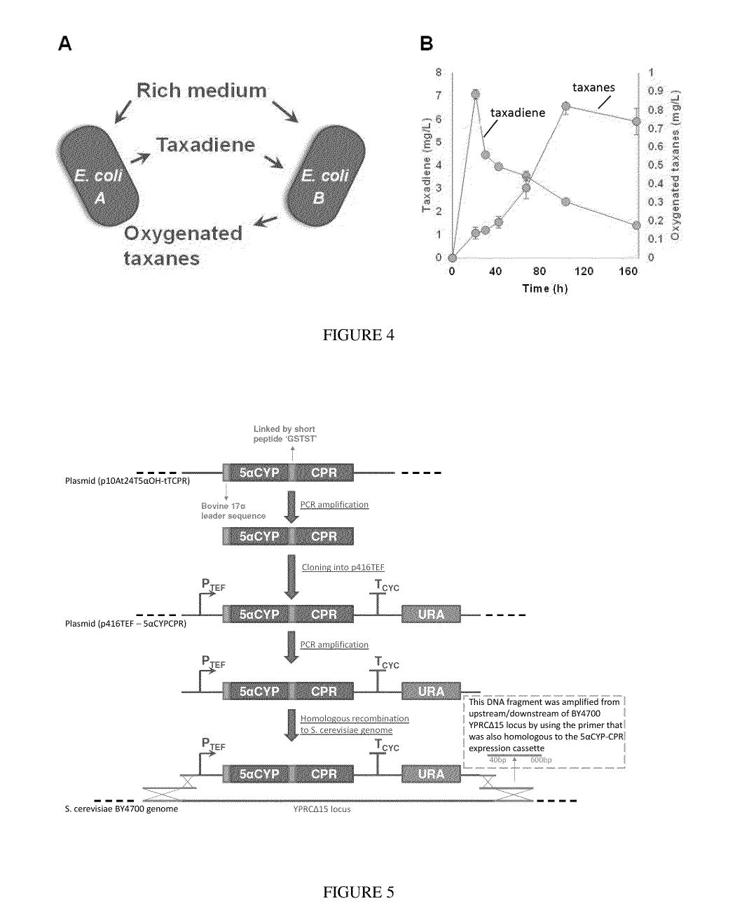

To make the cells function cooperatively, a mutualistic interaction was designed between the two microbes whereby each species benefits from the presence of the other (Nowak et al., 2006). It is known that E. coli can metabolize xylose and secrete acetate as a product, which inhibits its own growth (Xia et al., 2012). S. cerevisiae, on the other hand, cannot metabolize xylose but can utilize acetate as sole carbon source for growth without producing ethanol (FIG. 1B, FIG. 28). We thus switched the carbon source of the co-culture from glucose to xylose and, as predicted, S. cerevisiae grew in this xylose medium only in the presence of E. coli (FIG. 3A), while extracellular acetate in the co-culture was significantly lower than that in the E. coli culture (FIG. 3B). More importantly, this arrangement succeeded in maintaining ethanol concentration below detection limit (0.1 g/L) throughout the experiment. In addition, the titer of total taxanes produced by E. coli was not significantly affected by the presence of S. cerevisiae (FIG. 3C), suggesting that the ethanol inhibition of E. coli was successfully eliminated and taxadiene production proceeded unabated by the presence of yeast. The titer of oxygenated taxanes produced by the co-culture was also increased (FIG. 3D) as compared to the previous co-culture (2 mg/L in 72 hrs, FIG. 2A), but the taxadiene oxygenation efficiency was still low (only 8% of total taxadiene produced). After further optimization of bioreactor conditions (including the size of S. cerevisiae inoculum and maintaining sufficient carbon (xylose) and nitrogen (ammonium) sources for cell growth), 20 mg/L oxygenated taxanes were produced by the co-culture in 90 h (FIG. 3E).

A major goal of the co-culture concept is to introduce modularity in the design of pathways for microbial metabolite production by assigning a different part of the metabolic pathway to each member of the synthetic consortium. As such, pathway segments can be optimized separately and assembled together for optimal functioning of the overall pathway. To achieve this modular construction, pathway modules in different cells should not directly interact with each other to minimize feedback regulation. For example, CYPs and their reductase involved in taxane oxygenation generate reactive oxygen species (Pillai et al., 2011; Reed et al., 2011), which inhibit two enzymes (ISPG and ISPH) in the taxadiene biosynthetic pathway containing iron-sulfur clusters that are hyper-sensitive to ROS (Artsatbanov et al., 2012). Theoretically, spatial segregation of the pathway of taxadiene production from its oxygenation pathway should prevent inactivation of ISPG/ISPH by ROS generated by CYPs.

### Materials and Methods

E. coli Strains Used in the E. coli-S. cerevisiae Co-Culture

Four rate-limiting genes in the E. coli MEP pathway (dxs-idi-ispD-ispF) were previously cloned into araA locus of a modified E. coli MG1655, under control of a T7 promoter (FIG. 5). The resulting strain was named as MG1655_T7MEP. Codon optimized genes coding for Taxus Canadensis geranylgeranyl diphosphate synthase and Taxus brevifolia taxadiene synthase were cloned into lacY locus of MG1655_T7MEP as an operon, which was also controlled by a T7 promoter (unpublished data). The resulting strain (named as MG1655_MEP_TG) was used as taxadiene producing E. coli in the E. coli-S. cerevisiae co-culture. Strains used in these studies are summarized in Table 3.

S. cerevisiae Strains Used in the E. coli-S. cerevisiae Co-Culture

This section is illustrated in FIG. 5. The gene coding for fusion protein of taxadiene 5α-hydroxylase and its reductase was PCR amplified by using primers XbaI-bovine17a/CPR-his-HindIII (details of the primers used in this study have been summarized in Table 1). The plasmid p10At24T5αOH-tTCPR (Ajikumar et al, 2010)(FIG. 5) was used as template in this PCR reaction. The PCR product was digested by restriction enzymes XbaI/HindIII and cloned into XbaI/HindIII sites of p416-TEF (ATCC 87368) (using primers P24 and P25). The resulting plasmid containing the taxadiene 5α-hydroxylase expression cassette and the uracil marker was PCR amplified by using primers pBR322_origin_{—}607F/CEN6_{—}479F. The DNA fragments in the upstream and downstream of the YPRCΔ15 locus of S. cerevisiae genome were also PCR amplified by using primers YPRCΔ15_up/YPRCA15_up-p414 and p414-YPRCA15_down/YPRCΔ15_down respectively. The three PCR products were then co-transformed into S. cerevisiae BY4700 (ATCC 200866, MATa ura3Δ0), where the taxadiene 5α-hydroxylase expression cassette was integrated into the YPRCΔ15 locus via homologous recombination. The resulting strain (named as BY4700_SaCYPCPR, also referred to as TaxS1) was used to oxygenate taxadiene in the E. coli-S. cerevisiae co-culture.

E. coli Strains Used in the E. coli-E. coli Co-Culture

Strain EDE3Ch1TrcMEPp5T7TG (named TaxE10 in this study) was previously constructed (FIG. 5), and it was used for producing taxadiene in the E. coli-E. coli co-culture (Ajikumar et al., 2010). Plasmid p10At24T5αOH-tTCPR (FIG. 5) was transformed into E. coli MG1655ΔrecAΔendADE3 (a gift from Prof. Kristala Prather, MIT). The resulting strain (named as MG1655_{—}5aCYPCPR) was used to oxygenate taxadiene in the E. coli-E. coli co-culture.

### Characterization of the Yeast Cultures by Feeding Taxadiene.

All S. cerevisiae strains were characterized in absence of E. coli prior to co-culture experiment. We used 14 mL glass tubes (Pyrex) for this type of characterizations. A colony of the S. cerevisiae was inoculated into 1 mL YPD medium (10 g/L yeast extract, 20 g/L peptone, 20 g/L glucose) and grown at 30° C./250 rpm until cell density OD600 reached 2. Then, 10 μL of 6 g/L synthetic taxadiene stock solution (in DMSO) was added to start the experiments, and the cultures were then incubated at 22° C./250 rpm. To compare yeast growth and activity when growing on glucose or acetate, the same procedure was used except the medium was the one used in bioreactor experiments with indicated carbon source.

Bioreactor Experiments for the E. coli-S. cerevisiae Co-Culture

A 1 L Bioflo bioreactor (New Brunswick) was used for this study. Seed cultures of E. coli and S. cerevisiae were inoculated into 500 mL of defined medium (5 g/L yeast extract, 13.3 g/L KH₂PO₄, 4 g/L (NH4)₂HPO₄, 1.7 g/L citric acid, 0.0084 g/L EDTA, 0.0025 g/L CoCl₂, 0.015 g/L MnCl₂, 0.0015 g/L CuCl₂, 0.003 g/L H₃BO₃, 0.0025 g/L Na₂MoO₄, 0.008 g/L Zn(CH₃COO)₂), 0.06 g/L Fe(III) citrate, 0.0045 g/L thiamine, 1.3 g/L MgSO4, pH 7.0) containing 5 g/L yeast extract and 40 g/L glucose (or 20 g/L xylose). To prepare seed culture of E. coli, a colony of the E. coli was inoculated into Luria-Bertani (LB) medium (10 g/L tryptone, 5 g/L yeast extract, 10 g/L NaCl, pH=7) and grown at 37° C./250 rpm overnight. 5 mL of the grown cell suspension (OD of ˜6) was inoculated into the bioreactor. To prepare seed culture of S. cerevisiae, a colony of the S. cerevisiae was inoculated into YPD medium (10 g/L yeast extract, 20 g/L peptone, 20 g/L glucose) and grown at 30° C./250 rpm until cell density OD600 reached 20. 10 mL of the grown cell suspension were centrifuged at 3,000 g for 2 min, and pellets were resuspended in phosphate buffered saline (PBS) and inoculated into the bioreactor. In the control experiments, only E. coli or S. cerevisiae was inoculated into the bioreactor.

During the fermentation, oxygen was supplied by filtered air at 0.5 L/min and agitation was adjusted to maintain dissolved oxygen levels above 30%. The pH of the culture was controlled at 7.0 using 10% NaOH and 0.5M HCl. The temperature of the culture in the bioreactor was controlled at 30° C. until the dissolved oxygen level dropped below 40%. The temperature of the bioreactor was reduced to 22° C. and the E. coli was induced with 0.1 mM IPTG. During the course of the fermentation the concentration of glucose (or xylose), acetate and ethanol was monitored with constant time intervals. As the glucose concentration dropped below 20 g/L, 20 g/L of glucose was introduced into the bioreactor. As the xylose concentration dropped below 10 g/L, 10 g/L of xylose was introduced into the bioreactor.

To improve growth of the microbes, ammonium (nitrogen source) was also monitored with constant time intervals in the last experiment of the E. coli-S. cerevisiae co-culture (FIG. 3E). Ammonium phosphate was co-fed with xylose (1 g (NH₄)₂HPO₄ per 5 g xylose). As ammonium concentration dropped below 0.5 g/L, 4 g/L (NH4)₂HPO₄ was introduced to the bioreactor. In this experiment (FIG. 3E), more inoculum of the S. cerevisiae (pellets of 50 mL of grown cell suspension, OD600=20) was also used to minimize acetate accumulation, which indeed eliminated acetate accumulation (acetate concentration constantly <0.1 g/L).

Bioreactor Experiments for the E. coli-E. coli Co-Culture

A 1 L Bioflo bioreactor (New Brunswick) was used for this study. Half liter of rich medium (5 g/L yeast extract, 10 g/L tryptone, 10 g/L NaCl, 5 g/L K2HPO4, 8 g/L glycerol, pH7) containing 50 mg/L spectinomycin, was inoculated with 5 mL of grown culture (OD of 4) of E. coli EDE3Ch1TrcMEPp5T7TG (TaxE9) and 5 mL of grown culture (OD of 4) of E. coli MG1655_{—}5aCYPCPR (TaxE10). During the fermentation, oxygen was supplied by filtered air at 0.5 L/min and agitation was adjusted (280-800 rpm) to maintain dissolved oxygen levels above 20% (e.g., at 30%). The pH of the culture was controlled at 7.0 using 10% NaOH. The temperature of the culture in the bioreactor was controlled at 30° C. until the dissolved oxygen level dropped below 40%. The temperature of the bioreactor was reduced to 22° C. and the E. coli was induced with 0.1 mM IPTG. During the course of the fermentation, the concentration of glycerol and acetate was monitored with constant time intervals. Glycerol was fed into the bioreactor at the rate of 0.65 g/h.

Test Tube Experiments for Characterizing Acetate Production of E. coli.

A colony of E. coli was inoculated into LB medium, and incubated at 37° C./250 rpm overnight. 10 μL of grown cells were inoculated into the same medium as the one used in E. coli-S. cerevisiae bioreactors. The cell suspension was incubated at 22° C./250 rpm for 96 h and samples were taken for extracellular acetate measurement.

### Quantification of Isoprenoids, Including Taxanes

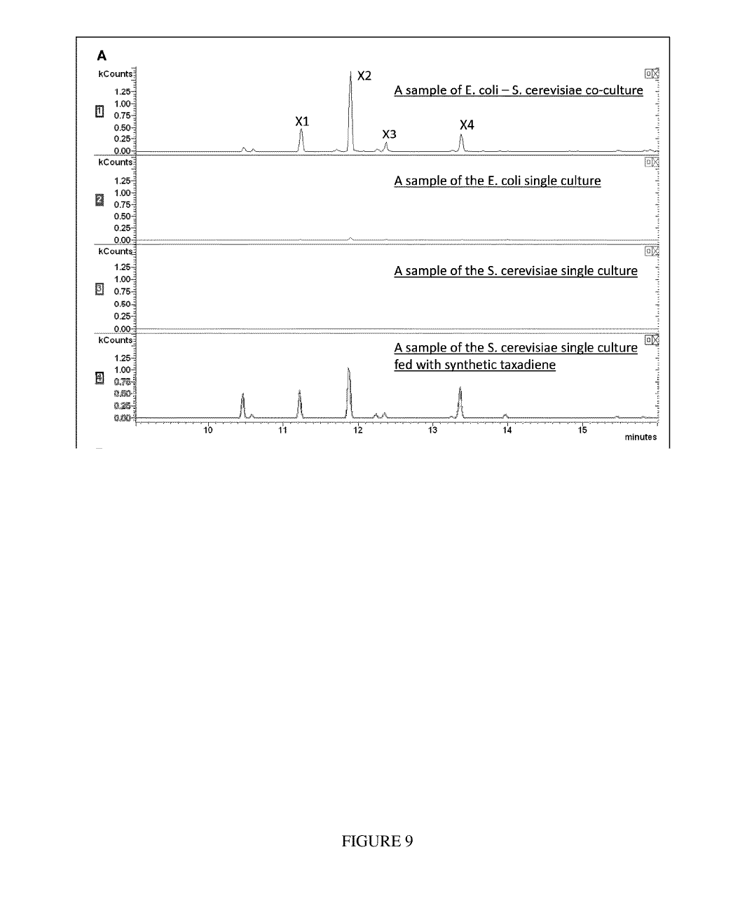

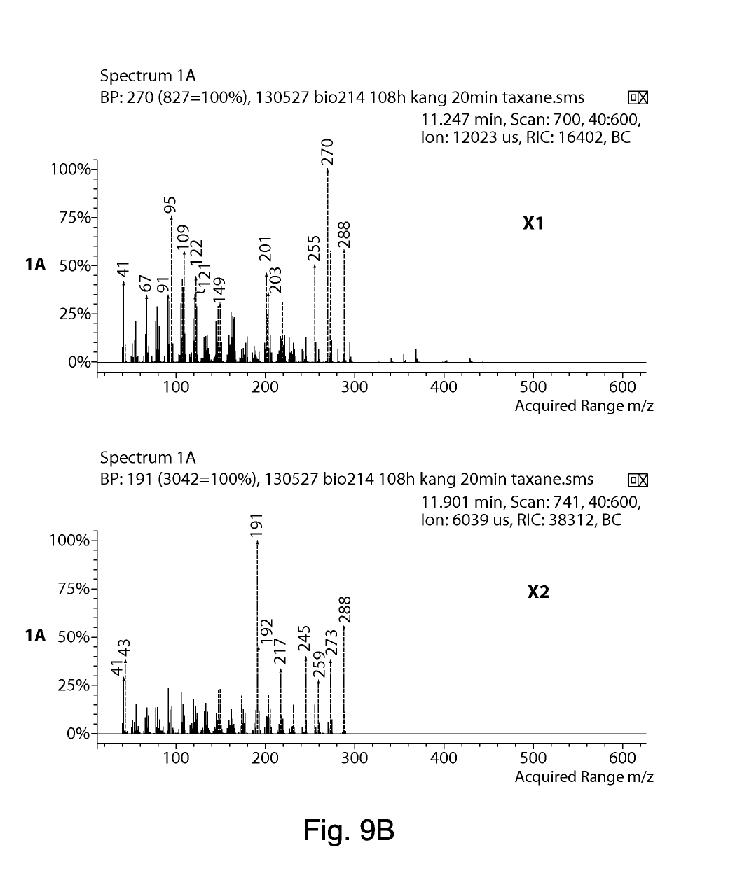

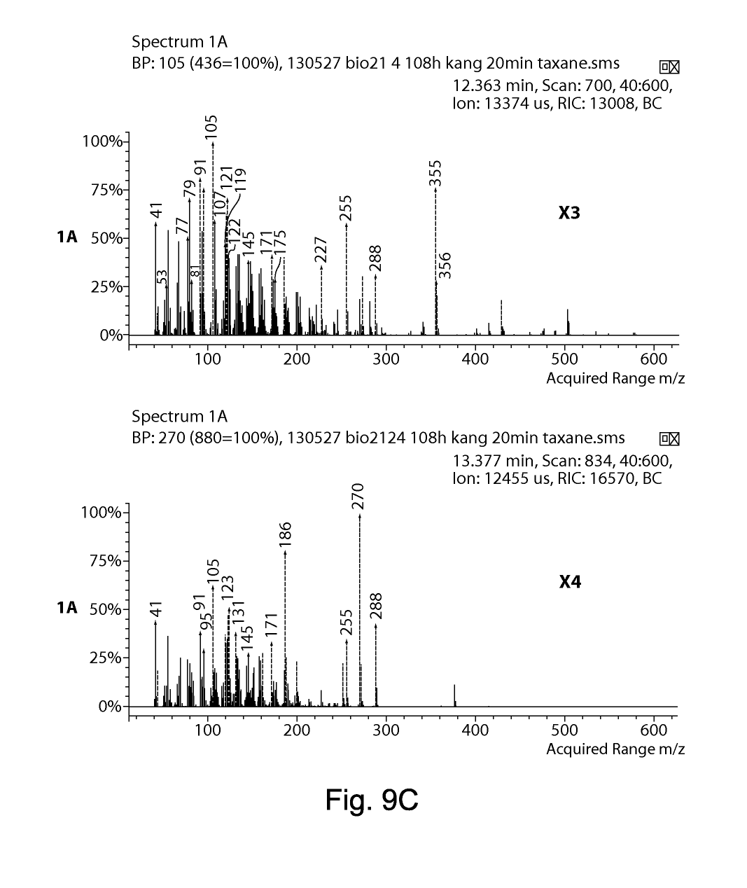

At indicated time points, 100 μL of cell suspension was sampled and mixed with 300 μL ethyl acetate and 100-200 uL 0.5 mm glass beads. Alternatively, 200 μL of cell suspension can be sampled and mixed with 200 μL ethyl acetate and 100 μL 0.5 mm glass beads. The mixture was vortexed at room temperature for 20 min, and clarified by centrifugation at 18,000 g for 2 min 1 μL of the ethyl acetate phase was analyzed by GCMS (Varian saturn 3800 GC attached to a Varian 2000 MS). The samples were injected into a HP5ms column (30 m×250 uM×0.25 uM thickness) (Agilent Technologies USA). Helium (ultra purity) at a flow rate 1.0 mL/min was used as the carrier gas. The oven temperature was kept at 100° C. for 1 min, then increased to 175° C. at the increment of 15° C./min, then increased to 220° C. at the increment of 4° C./min, then increased to 290° C. at the increment of 50° C./min and finally held at this temperature for 1 min. The injector and transfer line temperatures were both set at 250° C. The MS was operated under scan mode (40-600 m/z) and total ion count of taxanes was used for the quantification. Taxadiene, nootkatol and nootkatone were quantified using the calibration curve (total ion count vs. concentration) constructed with authentic standard. As standards of oxygenated taxanes were not available, oxygenated taxanes were also quantified by using the taxadiene calibration curve. Oxygenated taxanes were identified according to the characteristic m/z of mono-hydroxylated taxadiene (288 m/z, details are shown in FIGS. 9A-C).

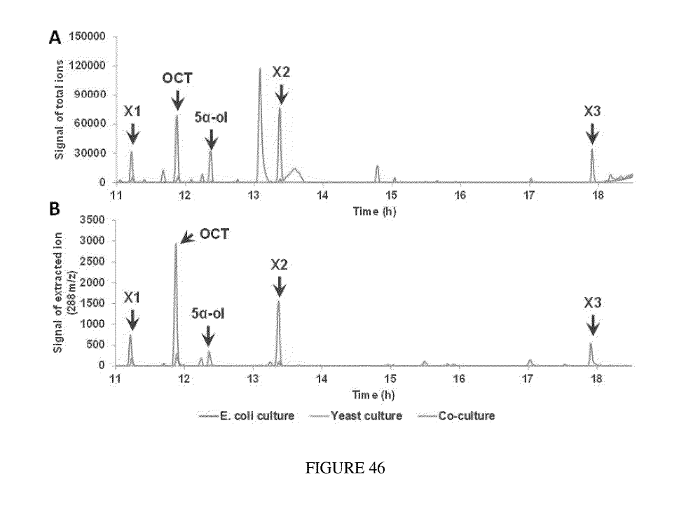

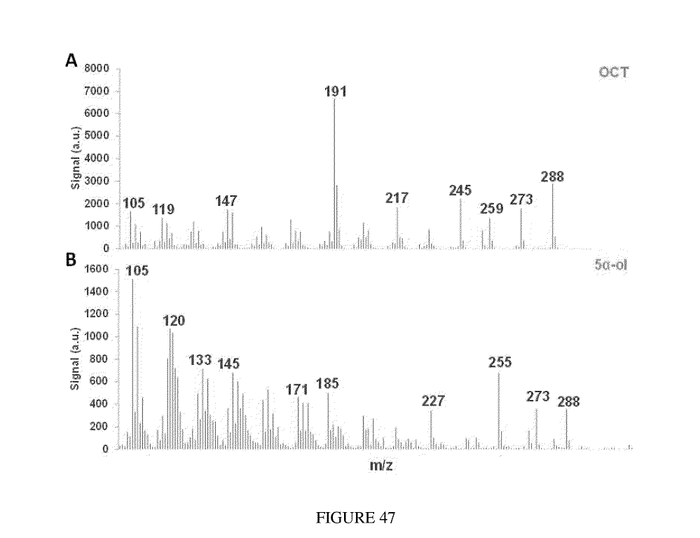

The 5αCYP was reported to produce multiple oxygenated taxanes in S. cerevisiae (Rontein et al., 2008). After analyzing co-culture samples, we also observed many peaks on total ion chromatography (40-400 m/z, GCMS) between 11-18.5 min, where we did not observe any peak when sample of the single cultures was analyzed (FIG. 46A). Five of the major peaks contained significant amount of 288 m/z signal (characteristic mass of monooxygenated taxane, 272 (taxadiene)+16 (oxygen) (FIG. 46A). Among them, two were previously identified as oxa-cyclotaxane (OCT) and taxadien-5α-ol (Ronstein et al., 2008) (FIG. 47). As a conservative estimate, we only considered these five oxygenated taxanes for calculating titer of total oxygenated taxanes. As standards of these five monooxygenated taxanes, the monoacetylated dioxygenated taxane and ferruginol were not available, they were quantified by using the taxadiene calibration curve.

### Quantification of Extracellular Metabolites

At indicated time points, 1.1 mL of cell suspension was sampled and centrifuged at 18,000 g for 1 min. The supernatant was sterilized by using 0.2 μm filter. 0.1 mL of filtered supernatant was analyzed by a Yellow Springs Instruments (YSI) 7100 (ammonium/potassium sensor) to measure extracellular ammonium concentration. 1 mL of filtered supernatant was analyzed a HPLC (Waters 2695 separation module coupled to Waters 410 differential refractometer) to measure concentration of extracellular glucose, xylose, acetate and ethanol. Bio-rad HPX-87H column was used and 14 mM sulfuric acid was used as mobile phase at the flow rate of 0.7 mL/min.

Quantification of E. coli and S. cerevisiae Cell Number

To measure cell number of viable E. coli in the E. coli-S. cerevisiae co-cultures, 2 μL of cell suspension was diluted in 200 μL sterile phosphate buffered saline (PBS), and 2 μL of the diluted cell suspension was further diluted in 200 μL sterile PBS. 50 μL of the repeatedly diluted cell suspension was plated on LB agar plate (1.5% agar) and incubated at 37° C. for 20 h. After the incubation, only E. coli colonies were visible on the plate (S. cerevisiae colonies cannot be formed at this condition because the growth temperature and carbon source are not ideal for its growth). The yeast colonies were only visible after at least 48 hrs in these conditions.

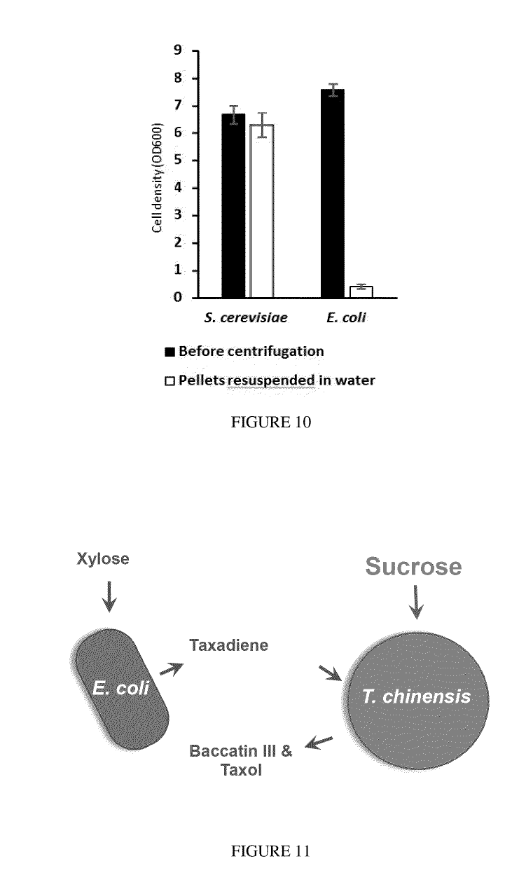

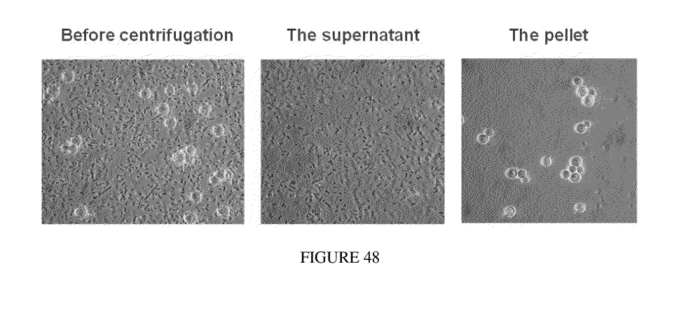

To estimate cell number of S. cerevisiae in the E. coli-S. cerevisiae co-cultures, S. cerevisiae was separated from the mixed culture by centrifugation at 100 rpm for 1 min (Beckman coulter microfuge 18). As shown in FIG. 10 only S. cerevisiae can be efficiently centrifuged at this speed. The pellet containing mostly S. cerevisiae was resuspended in water and optical density 600 of the resuspended cells was measured (FIG. 48). After this separation, cell number of the two microbes could be quantified by measuring optical density at 600 nm.

Table 1 presents primers used in the example

| Primer | Sequence |
|---|---|
| XbaI-bovine17a | GCtctagaAAAATGGCTCTGTTATTAGCAGTT |
| | (SEQ ID NO: 1) |
| CPR-his-HindIII | GCaagcttTTAgtgatggtgatgatgatgCCA |
| | AATATCCCGTAAGTAGC (SEQ ID NO: 2) |
| YPRCΔ15_up | GCCAGGCGCCTTTAT (SEQ ID NO: 3) |
| YPRCΔ15_up-p14 | gcaaaaggccaggaaccgtaaaaaggccgcgt |
| | tgctggcgtTTTGCGAAACCCTATGC |
| | (SEQ ID NO: 4) |
| p414-YPRCΔ15_ | ggacggatcgcttgcctgtaacttacacgcgc |
| down | ctcgtatcAATGGAAGGTCGGGATG |
| | (SEQ ID NO: 5) |
| YPRCΔ15_down | ATAAAGCAGCCGCTACC (SEQ ID NO: 6) |
| pBR322_origin_ | ACGCCAGCAACGCGG (SEQ ID NO: 7) |
| 607F | |
| CEN6_479F | GATACGAGGCGCGTGT (SEQ ID NO: 8) |

### Example 2

Methyl jasmonate (MeJA) induction is able to induce paclitaxel synthesis in Taxus sp. suspension cells (Li et al., 2012). Although MeJA induction does result in transcriptional up-regulation of the cytochrome P450s and other enzymes which functionalize taxadiene, MeJA treatment also leads to concurrent down-regulation of the taxadiene synthetic pathway (Li et al., 2012). Thus, availability of taxadiene in the plant cells may be restricting the paclitaxel production in plant cell culture.

In effort to harness the efficient taxadiene oxygenation capacity of MeJA-induced Taxus cells but circumvent the limitation of taxadiene availability in these cells, a synthetic cellular consortium is established using Taxus cells and E. coli cells (FIG. 11). To form the co-culture, taxadiene-producing E. coli are inoculated to a culture of Taxus chinensis cells that are induced with MeJA to up-regulate cytochrome P450 and other enzymes (FIG. 11).

In some cases, there is competition within the two species of cells of the consortium for a carbon source. This can lead to overgrowth of the culture with E. coli cells due to their fast growth rate. To maintain a defined ratio of E. coli and the T. chinensis cells, a medium is used that contains both xylose and sucrose, which can exclusively be utilized by the E. coli and the T. chinensis cells respectively. Controlling availability of xylose and sucrose allows maintenance of a stable co-culture of the two species. Taxadiene produced by E. coli serves as an exogenous taxadiene source for T. chinesis cells that are able to internalize and functionalize the intermediate to Baccatin III and Taxol (FIG. 11). To further increase efficiency of the consortium and mass transfer of taxadiene between two cells, lipids that can be taken up by the Taxus suspension cells can be supplied to the culture as taxadiene carriers.

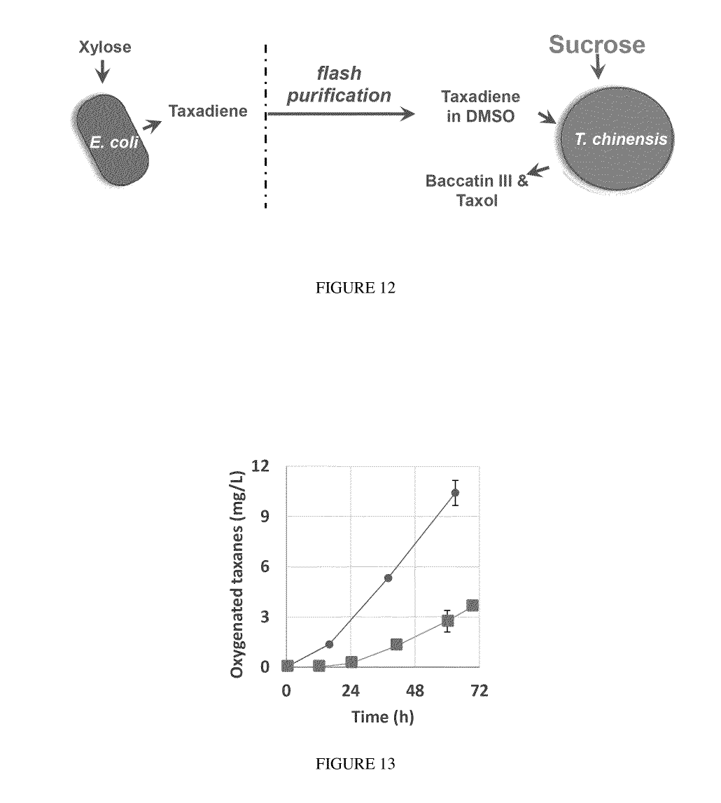

In some cases, one or both species of the co-culture cannot grow optimally in the co-culture conditions. To circumvent this challenge, the cellular consortium can be cultured in separate environments (FIG. 12). Taxadiene-producing E. coli are grown in medium containing xylose that supported bacterial growth and synthesis of the intermediate compound. Taxadiene is isolated from the culture, flash purified, and used to supplement the MeJA-induced T. chinensis culture. In its own optimal conditions, T. chinensis internalizes the taxadiene and further functionalizes the compound to efficiently produce Baccatin III and Taxol (FIG. 12).

### Example 3

After the surprising success of the co-culture system in producing oxygenated taxanes, components of the system, as well as the process of the system, were further optimized to increase production of the final product. As demonstrated in FIG. 3D, a mutualistic co-culture was achieved, although the oxygenation efficiency of S. cerevisiae could be improved. Thus, several measures were taken to improve the process, including increasing the amount of S. cerevisiae used to inoculate the co-culture and supplying additional nutrients to the culture at 41 hrs. Prior to inoculation, S. cerevisiae was grown in YPD medium until the cells reached an optical density at 600 nm (OD₆₀₀) of 20. Increasing the inoculum volume from 10 mL to 50 mL of the OD₆₀₀ 20 culture as well as providing additional nutrients to the culture resulted in a 3-fold increase in oxygenated taxane titer and no residual detectable acetate in the culture (FIG. 13).

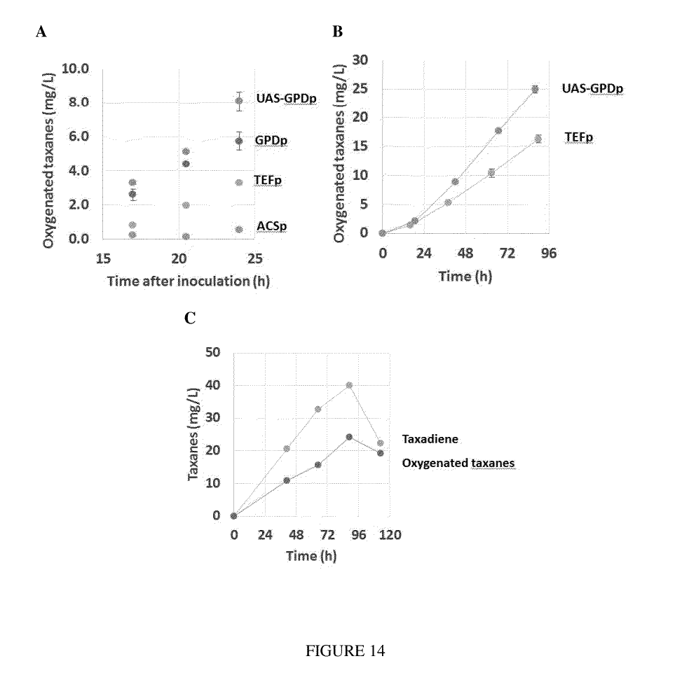

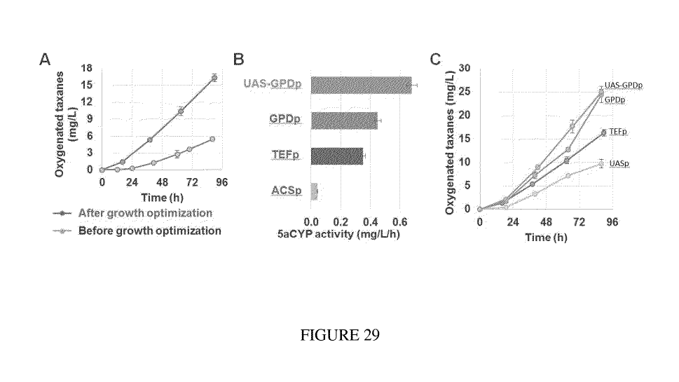

In addition to process improvement, the system can be further genetically engineered to increase production of oxygenated taxanes. To increase functionalization of taxanes in S. cerevisiae, the expression of the taxadiene 5α hydroxylase and NADPH-cytochrome P450 reductase (5αCYP-CPR, fused as a single polypeptide) was modulated by replacing the promoter sequence (FIG. 14A, FIG. 33A). S. cerevisiae was initially genetically modified to encode the taxadiene 5α hydroxylase and NADPH-cytochrome P450 reductase under control of the translation elongation factor 1a (TEF) promoter (TEFp). Substitution of the TEF promoter with the glyceraldehyde-3-phosphate dehydrogenase (GPD) promoter (GPDp), the glyercaldehyde-3-phosphate dehydrogenase promoter including the upstream activation sequence elements (UAS-GPDp), and ACSp (a promoter from the acetate assimilation pathway (De Virgilio et al., 1992; Kratzer et al., 1997). The efficiency of taxadiene oxygenation was tested by the corresponding strains. Both GDPp and UAS-GPDp resulted in increased production of oxygenated taxanes, whereas replacement with the acyl-coenzyme A synthetase (ACS) promoter resulted in a decrease (FIG. 14A). The strains were also cultured without E. coli and the oxygenation rate of exogenously supplied taxadiene was measured (FIG. 29B

Because it contained the best-performing promoter, S. cerevisiae expressing the taxadiene 5α hydroxylase and NADPH-cytochrome P450 reductase under control of the UAS-GPD promoter was expressed in strain TaxS4 and was selected for co-culture with taxadiene-producing E. coli (TaxE1). Use of the UAS-GPD promoter resulted in production of 60% more oxygenated taxanes compared to S. cerevisiae with the TEF promoter (FIG. 14B). Further analysis of the total taxanes produced by S. cerevisiae with the UAS-GPD promoter revealed that more than 50% of the taxanes were taxadiene rather than the desired product, oxygenated taxanes (FIG. 14C).

These results indicated there was a limitation of the system, presumably the growth or metabolism of S. cerevisiae which is dependent on the availability of acetate. It was noted that acetate accumulated in the co-culture during the first 24 h (FIG. 3B), indicating that the initial yeast population was insufficient to convert all available substrate in the medium. This was corrected by increasing the initial inoculum of yeast and also periodically feeding additional carbon (xylose), nitrogen (ammonium) and phosphorous (phosphate) sources to ensure that these major nutrients were not limiting yeast growth. After these modifications, no acetate was detected throughout the entire fermentation and the oxygenated taxane titer was improved ˜3 fold (16 mg/L in 90 h, FIG. 29A). At this condition, as growth of S. cerevisiae was strictly limited by the amount of acetate secreted by E. coli, further increase of the relative amount of yeast in the culture relied on engineering the acetate pathway in E. coli (see below). We opted to not feed exogenous acetate in order to preserve the autonomous nature of the co-culture (FIG. 34).

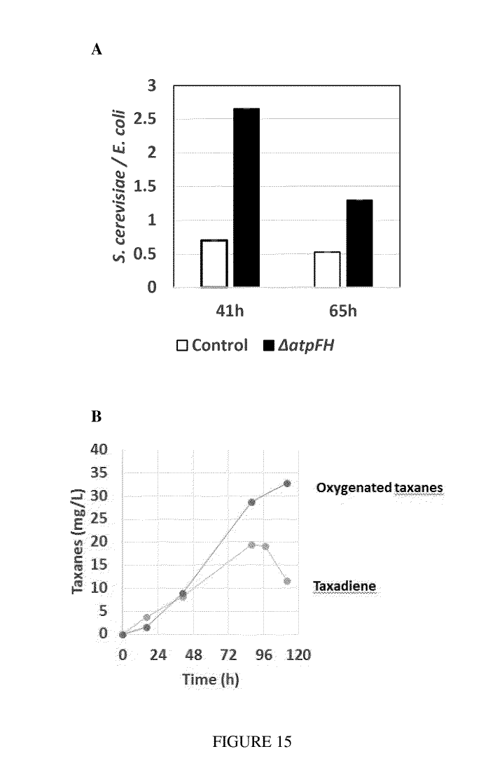

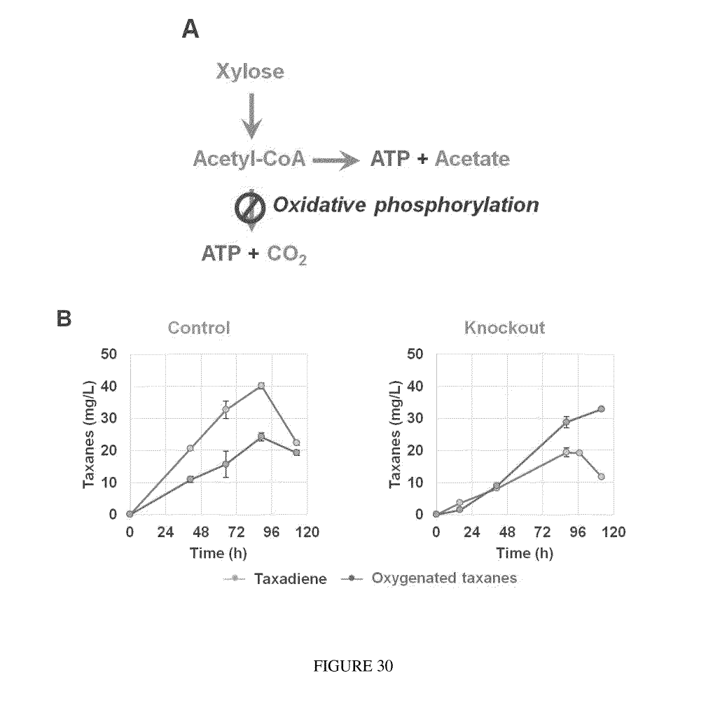

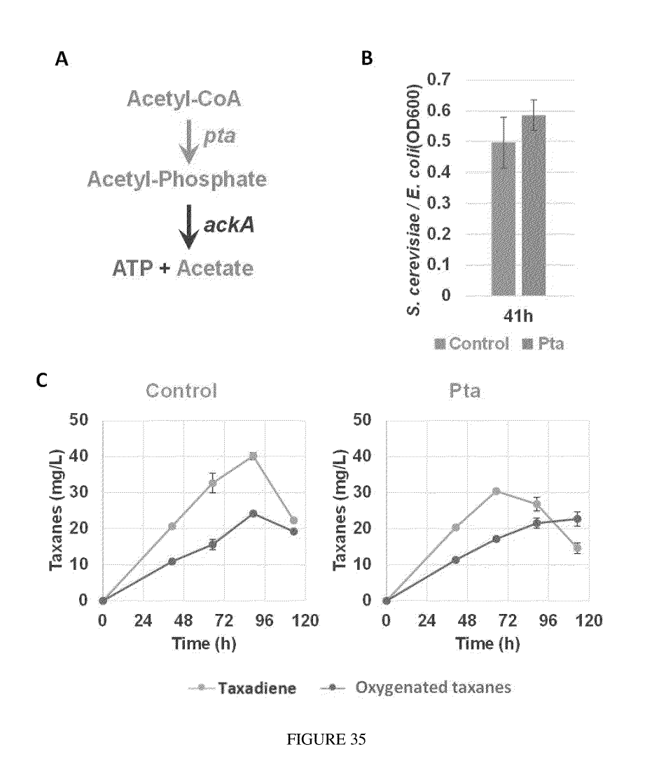

To further improve S. cerevisiae growth and metabolism, the taxadiene-producing E. coli was engineered to over-produce acetate. Production of acetate by E. coli is auto-regulated: when acetate accumulates, E. coli growth is inhibited, resulting in lower acetate production. To over-produce acetate, first, genes in the E. coli acetate production pathway (phosphate acetyltransferase, pta, and acetate kinase, ackA) were overexpressed. This neither increased the S. cerevisiae population nor the oxygenation efficiency significantly (FIG. 35). To overcome this regulation, oxidative phosphorylation was inactivated by the deletion of the membrane bound F₁F₀ H⁺-ATP synthase subunits encoded by atpFH. (FIG. 15A, FIG. 30A). Not only was the ratio of S. cerevisiae cells to E. coli cells higher when using E. coli ΔatpFH in the co-culture (FIG. 15A), but it also resulted in a higher portion of oxygenated taxanes (up to 75%) and higher titer of oxygenated taxanes (33 mg/L in 120 h), indicating a more efficient utilization of taxadiene (FIG. 15B, FIG. 30B).

### Materials and Methods

E. coli Strains Used in the E. coli-S. cerevisiae Co-Culture

To engineer E. coli TaxE1 to overproduce acetate, the pta or pta-ackA operon was overexpressed by using a pSC101 based plasmid containing trc promoter (p5trc1). Pta or ackA amplified from E. coli MG1655 chromosome was assembled with part of p5trc by using the recently developed Cross-Lapping In Vitro Assembly (CLIVA) method (primer P1-P6 used), yielding plasmid p5trc-pta and p5trc-ackA respectively (Zou et al., 2013). Primers used in this study are summarized in Table 4. All the plasmids constructed in this study were validated via sequencing. Plasmid p5trc-pta was transformed into E. coli TaxE1, described in Example 1, yielding E. coli TaxE2. AckA with trc promoter and terminator was amplified from p5trc-ackA and cloned into p5trc-pta via CLIVA (primer P7-P10 used), yielding plasmid p5trc-pta-trc-ackA. This plasmid was transformed into E. coli TaxE1, yielding E. coli TaxE3. After overexpression of pta and pta-ackA, oxidative phosphorylation of E. coli TaxE1 was inactivated by knocking out atpFH as described previously (primer P11 and P12 used), yielding E. coli TaxE4 (Causey et al., 2003).

S. cerevisiae Strains Used in the E. coli-S. cerevisiae Co-Culture

To replace the TEFp with GPDp and ACSp, GPDp amplified from plasmid p414-GPD (ATCC 87356) or ACSp amplified from BY4700 chromosome was combined with part of pUC-YPRC15-URA-TEFp-5αCYP-CPR via CLIVA (primers P34-P41 were used), yielding plasmid pUC-YPRC15-URA-GPDp-5αCYP-CPR-CYCt and pUC-YPRC15-URA-ACSp-5αCYP-CPR-CYCt, respectively. These two plasmids were linearized by using NotI and transformed into BY4700 (YPRC15 locus), yielding yeast TaxS2 and TaxS3 respectively (Flagfeldt et al., 2009). To add upstream activation sequence (UAS) to GPDp, the UASTEF-UASCIT1-UASCLB223 was synthesized (as gblock gene fragment, Integrated DNA Technologies) and cloned into pUC-YPRC15-URA-GPDp-5αCYP-CPR-CYCt via CLIVA (primer P42-P45 used), yielding pUC-YPRC15-URA-UAS-GPDp-5αCYP-CPR-CYCt. This plasmid was linearized by using NotI and transformed into BY4700 (YPRC15 locus), yielding yeast TaxS4. Sequences of all the synthetic genes used in this study are summarized in Table 5.

### Example 4

### Expression of the Muconic Acid Biosynthetic Pathway in a Single E. coli Cell

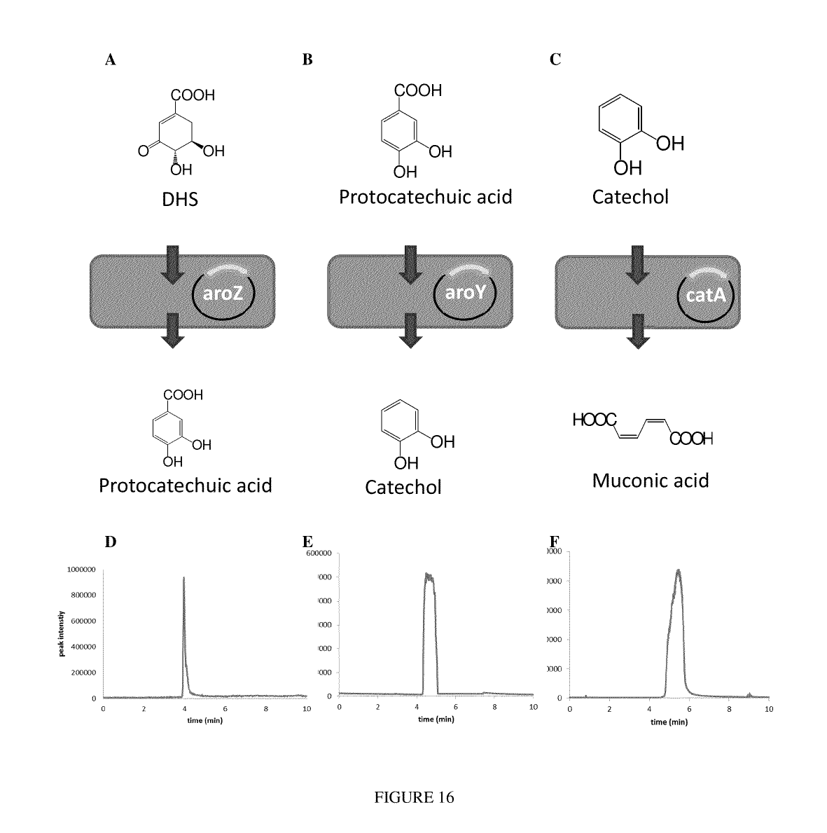

Heterologous gene candidates encoding enzymes involved in the production of muconic acid from dehydroshikimate (DHS) were identified in different organisms. Each gene was cloned into an expression vector for recombinant expression in E. coli. Each candidate enzyme expressed in E. coli was screened for optimal activity. As shown in FIGS. 16A and 16D, expression of the DHS dehydratase AroZ resulted in the conversion of DHS to protocatechuic acid (PCA). As shown in FIGS. 16B and 16E, expression of the PCA decarboxylase AroY resulted in the conversion of PCA to catechol. Finally, as shown in FIGS. 16C and 16F, expression of the catechol 1, 2-dioxygenase CatA resulted in conversion of catechol to muconic acid. The genes encoding aroZ and aroY were isolated from Klebsiella pneumoniae. The gene encoding catA was isolated from Acinetobacter calcoaceticus.

Having demonstrated the ability of each of the enzymes to perform its predicted function, three of the genes for muconic acid synthesis, aroZ, aroY, and catA, were recombinantly expressed in E. coli strain rpoA14, a strain that was previously engineered to overproduce tyrosine (Santos et al., 2012).

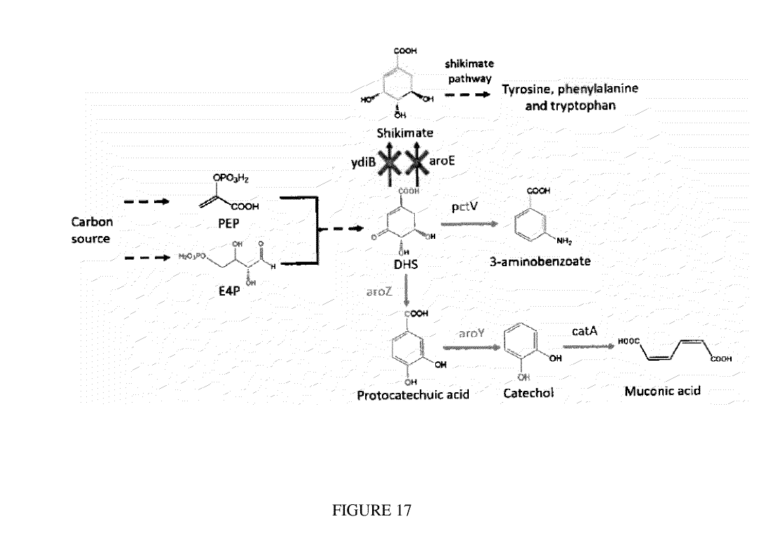

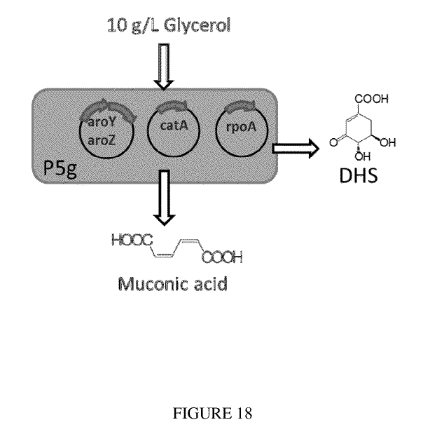

DHS, the substrate for muconic acid biosynthesis, can also be utilized in a cell in a competing pathway for the production of shikimate and aromatic amino acids. To reduce the flux of DHS away from the production of shikimate and aromatic amino acids and towards the recombinant biosynthetic pathway and production of muconic acid, genes of the competing pathway (ydiB and aroE) were knocked out (FIG. 17), resulting in generation of the strain E. coli P5g (FIG. 18A). This strain as also engineered to contain a plasmid that expresses a mutated global transcription machinery protein, RpoA.

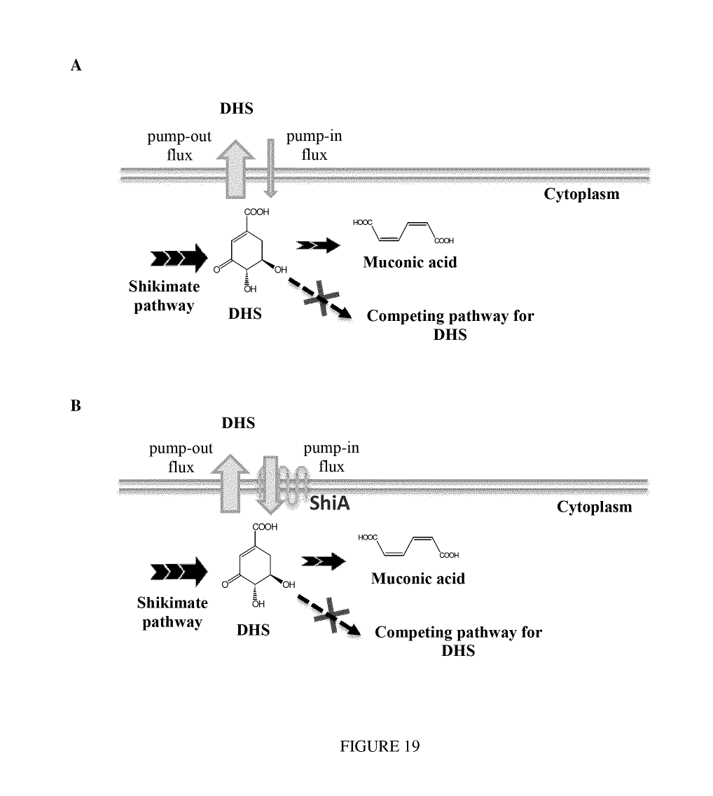

E. coli strains KM and P5g were cultured in the presence of 10 g/L glycerol as the carbon source and the biosynthesis of muconic acid, catechol, PCA and DHS was assessed by liquid chromatography-mass spectrometry after 4 days of cultivation on glycerol (FIG. 21). Reconstituting the muconic acid biosynthesis pathway in E. coli strain KM only resulted in the production of 28 mg/L muconic acid in the test tube. Deletion of the competing pathway from E. coli metabolism in strain P5g improved the muconic acid titer to approximately 270 g/L muconic acid. Surprisingly, it was found that the intermediate DHS was efficiently exported and accumulated to a relatively high titer in the supernatant of the culture during the biosynthesis process (FIGS. 19A and 21).

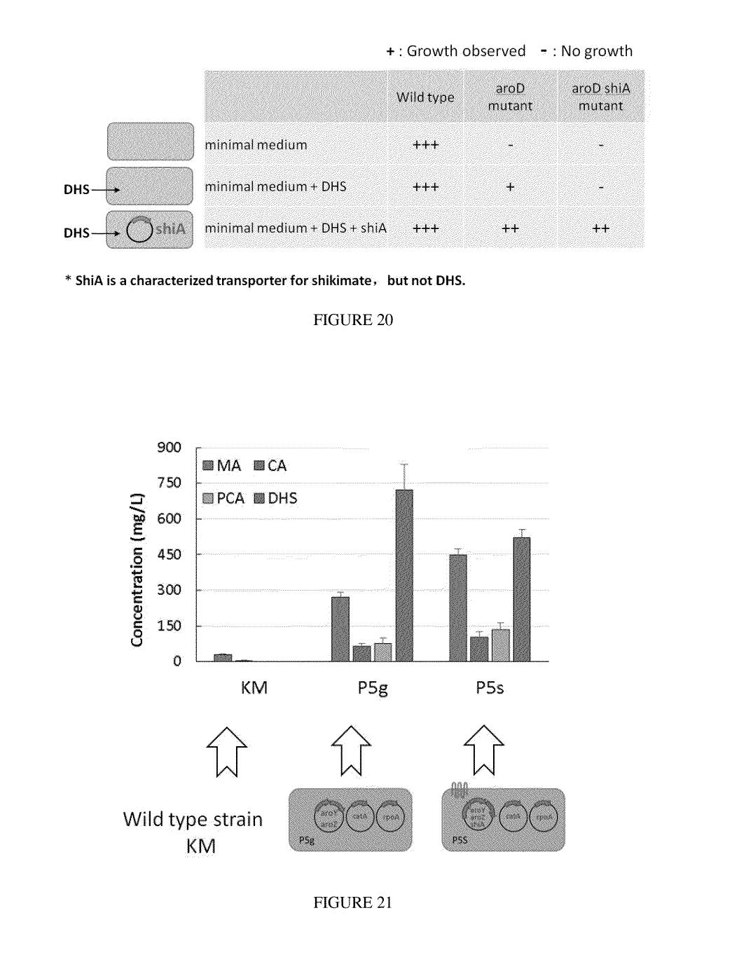

To overcome this issue and increase the flux of DHS into the cell for improved efficiency in using the abundantly available substrate, potential transporters for DHS were explored. The transmembrane permease ShiA is a characterized transporter for shikimate, though its potential for transporting DHS has not been evaluated. The E. coli permease shiA was cloned into an over-expression vector and transformed into E. coli strain deficient in aroD and thereby unable to produce DHS. This strain was tested for its ability to import DHS (FIG. 19B). As shown in the FIG. 20, the over-expression of ShiA in combination with exogenous DHS was able to rescue growth of an E. coli mutant lacking aroD and shiA expression, indicating ShiA is also a DHS transporter.

ShiA was then expressed to facilitate the DHS importation and to improve the muconic acid production by expressing the permease in P5g, resulting in the generation of E. coli strain P5S. E. coli strains KM, P5g, and P5S were cultured in the presence of 10 g/L glycerol as the carbon source and the biosynthesis of muconic acid, catechol, PCA and DHS was assessed by liquid chromatography-mass spectrometry after 4 days of cultivation on glycerol. As compared to E. coli strain P5g that did not express ShiA, the over-expression of the importer ShiA in E. coli strain P5S resulted in a decrease in DHS accumulation as well as a 40% improvement in muconic acid production, approximately 500 mg/L muconic acid from 10 g/L glycerol in a test tube (FIG. 21). These results indicated the single cell expression system was functioning at 9% of the theoretical maximum yield of the system.

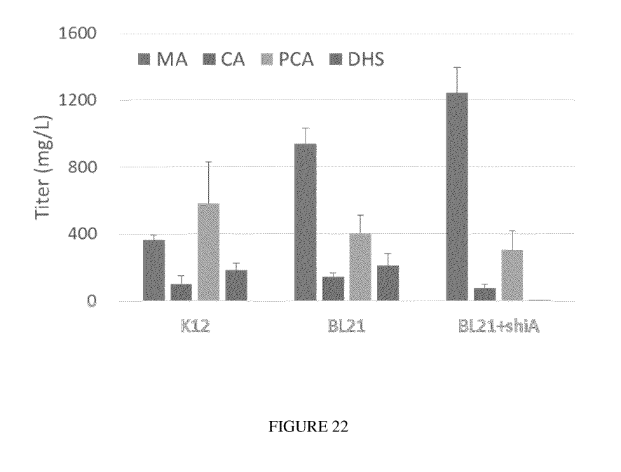

Different E. coli strains were also tested for their ability to express the enzymes of the recombinant pathway and produce muconic acid. E. coli K12 and E. coli BL21 (DE3) were engineered to express aroY, aroZ, and catA. E. coli BL21 (DE3) was further engineered to also express ShiA (BL21+shiA). Each of the strains was cultures in the presence of 2 g/L exogenous DHS and production of muconic acid, catechol, PCA and DHS was assessed. As shown in FIG. 22, E. coli BL21 (DE3) was found to be a better host for expression of the downstream biosynthetic genes compared to E. coli K12.

### Co-Culture for the Production of Muconic Acid

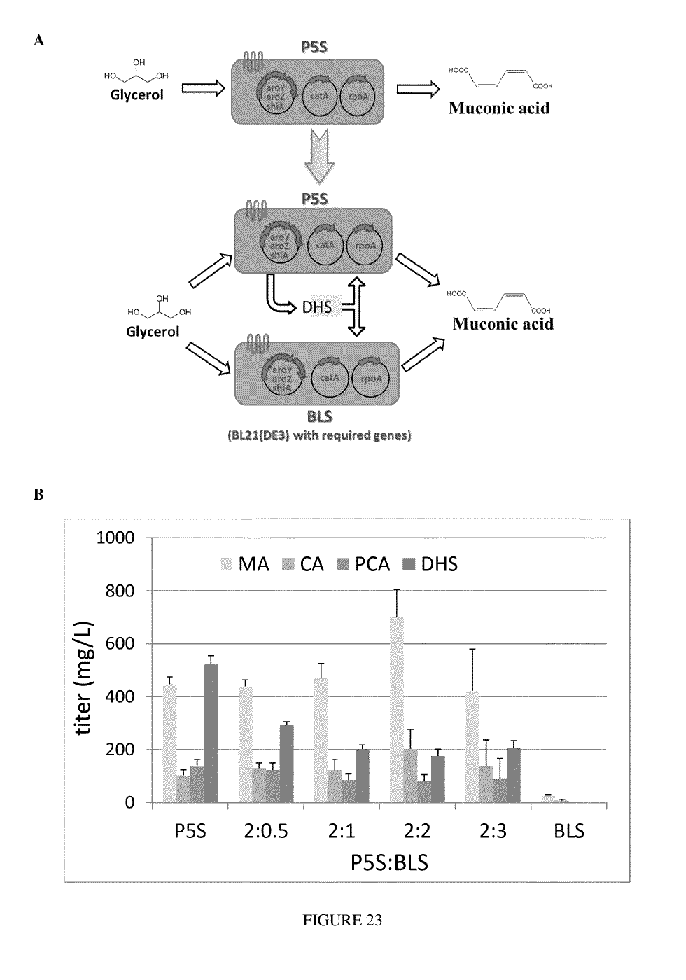

To further reduce the DHS intermediate accumulation in the supernatant, a two strain co-culture approach was employed. The E. coli strain P5S used in the single strain studies was co-cultured in the presence of a second E. coli strain, BLS, that expresses the genes for importing DHS and converting DHS into muconic acid, including shiA, aroZ, aroY, and catA (FIG. 23A). In such a co-culture system, the DHS intermediate that is produced and secreted by the first cell can be utilized by the second cell to enhance muconic acid production levels. The initial ratio of the two cells (P5S:BLS) was further varied to achieve optimal muconic acid titers. Following co-culture, production of muconic acid, catechol, PCA and DHS was assessed (FIG. 23B). An initial ratio of 2:2 resulted in the highest muconic acid titers and lowest DHS titers, indicating the system was functioning efficiently to utilize available substrates.

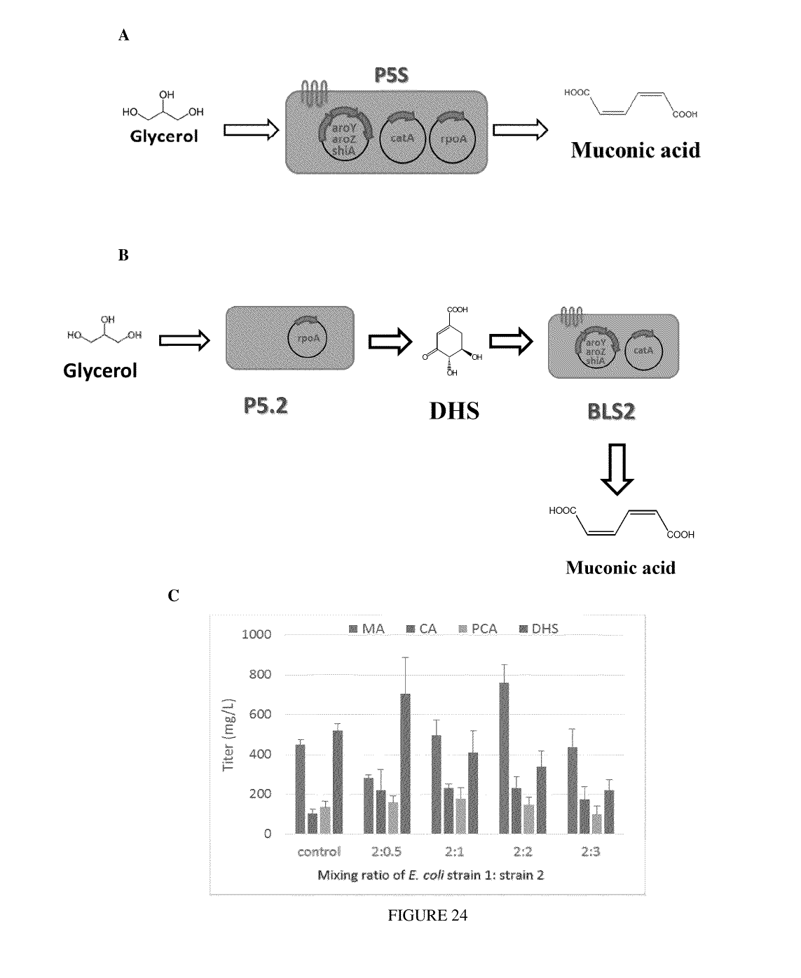

To further explore the potential of the co-culture strategy, a modular engineering approach was taken to divide the biosynthetic pathway into two modules, each of which was expressed in a distinct E. coli strain (FIG. 24B). The first module/strain (P5.2) was engineered produce the intermediate DHS from simple carbon sources. The second strain (BLS2) was engineered to import DHS produced by the first strain and convert the intermediate into muconic acid. This modular approach reduces the metabolic burden on each individual cell, as each cell is only responsible for half of the biosynthetic pathway. Furthermore, any detrimental interference between the upstream and downstream modules (e.g., feedback inhibition) is eliminated by physically separating the modules in distinct cells.

The two strains were co-cultured together at varying ratios in the presence of glycerol as a carbon source, then the production of muconic acid, catechol, PCA and DHS was assessed. As shown in FIG. 24C, dividing the biosynthetic pathway into two strains resulted in improved muconic acid production to nearly 800 mg/L from 10 g/L glycerol and also reduced the amount of DHS in the supernatant. These results indicated the modular co-culture system was functioning at 12% of the theoretical maximum yield of the system.

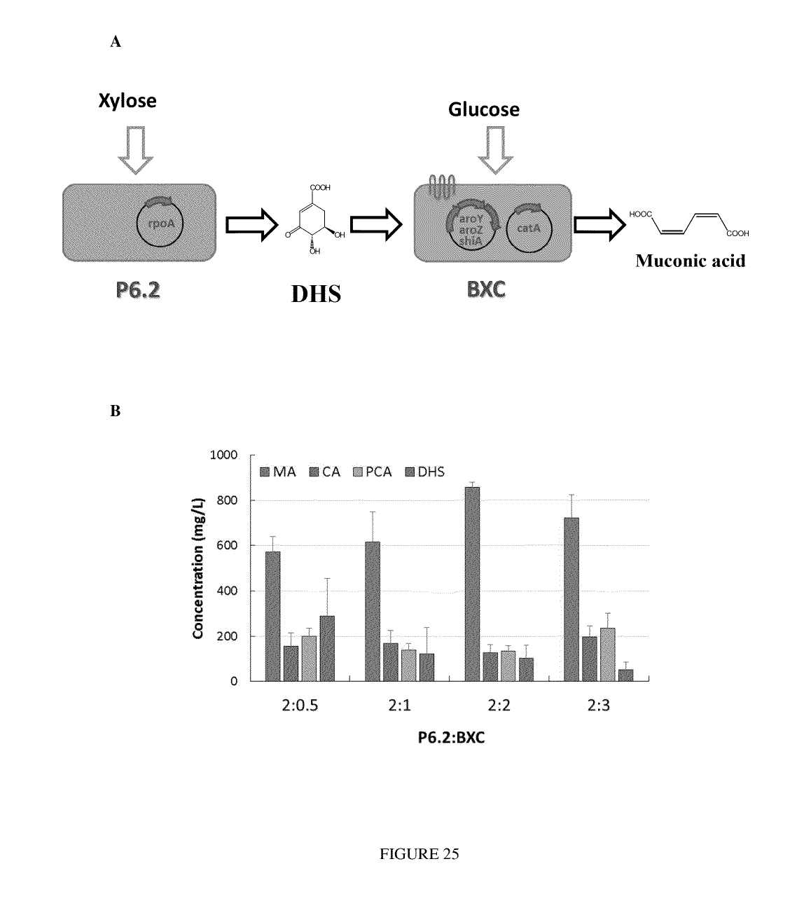

Simultaneous carbon source uptake is difficult to achieve due to the catabolite repression effect in which catabolism of one carbon source inhibits the catabolism of other carbon sources. Each strain was then further engineered to utilize a different carbon source in the co-culture environment to eliminate competition between the strains for a single carbon source (FIG. 25A). The glucose import system was knocked-out from the first strain (P6.2); consequently, the first strain was not able to consume glucose. The xylose utilization pathway was disrupted in the second strain (BLC), resulting in a second strain that did not consume xylose. The two strains were co-cultured in the presence of a mixture of 6.6 g/L glucose and 3.3 g/L xylose. The production of muconic acid, catechol, PCA and DHS was assessed (FIG. 25B). By optimization of the co-culture system through modulating carbon utilization, the system produced 300 mg/L muconic acid from a mixture of glucose and xylose, which are the major components of the naturally abundant and renewable biomass resource lignocellulose. These results indicated that the modular co-culture system was functioning at 9% of the theoretical maximum yield of the system.

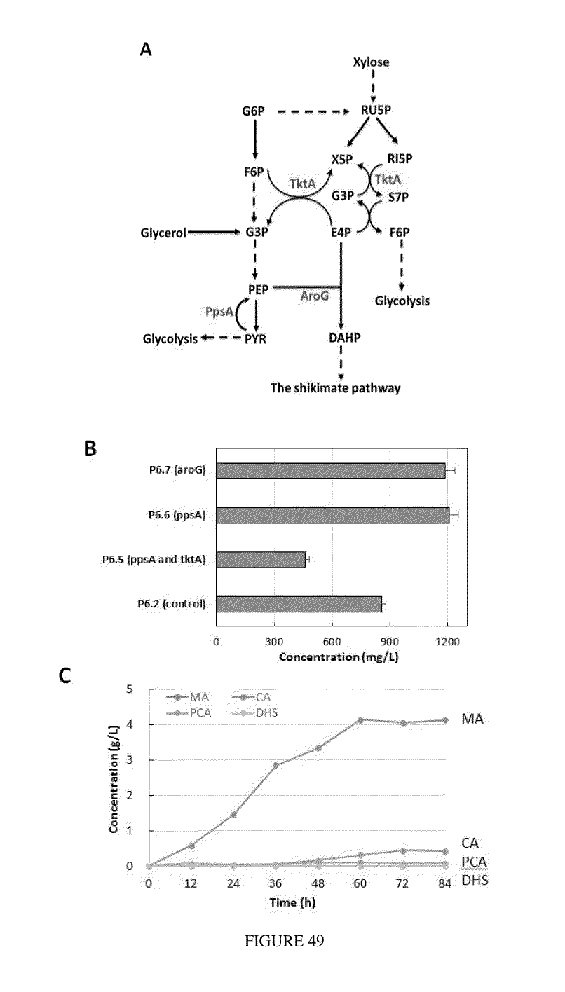

The production of muconic acid was further improved by over-expression of the upstream pathways, particular aroG and ppsA (FIG. 49A). The resulting new strain, referred to as P6.6, was able to be co-cultured with the BLC strain to produce 1.2 g/L muconic acid (FIG. 49B). When high cell density cultivation was performed, co-culture of strains P6.6 and BLC were able to produce 4 g/L muconic acid from 13.4 g/L glucose and 6.6 g/L xylose, which corresponded to 20% mass yield (FIG. 49C). This yield is higher than any previously reported system.

### Example 5

### Co-Culture for the Production of PHB

A modular co-culture system can also be utilized to produce other aromatic compounds derived from DHS. PHB is a native E. coli metabolite, whose biosynthesis uses the shikimate pathway including the intermediate DHS.

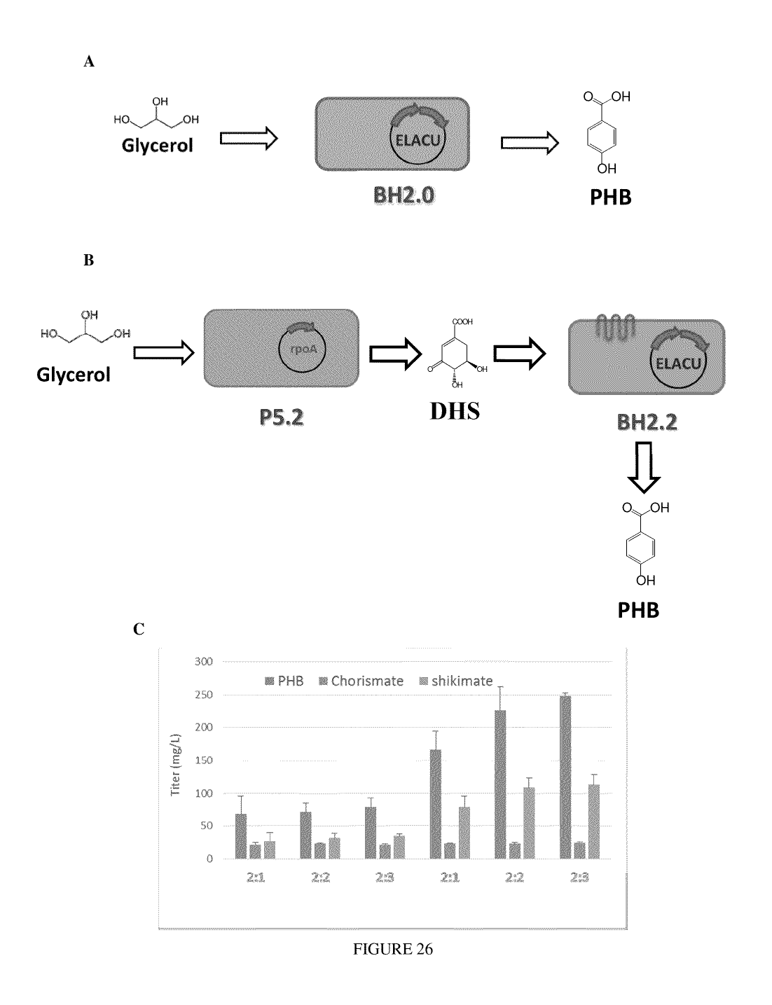

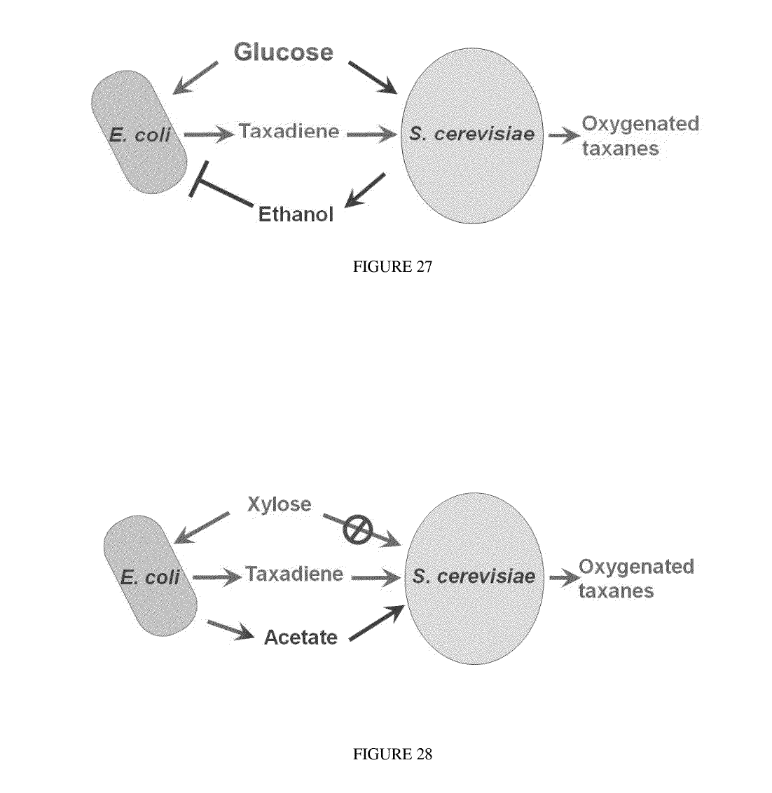

The biosynthetic pathway for the production of PHB from DHS was recombinantly expressed in a single cell (FIG. 26A), as well as divided into more than one cell (FIG. 26B). To engineer a modular co-culture system for the production of PHB, the same first strain/module that secretes the DHS intermediate (P5.2) as described in Example 4 was used. The second strain/module (BH2.2) was engineered to import DHS produced by the first cell by over-expressing ShiA and then convert DHS to PHB through recombinant expression of aroE, aroL, aroA, aroC and ubiC (FIG. 26B). The strains were co-cultured after which production of PHB, chorismate and shikimate were assessed (FIG. 26C). PHB was produced by the co-culture system at a level of 75 mg/L in the absence of ShiA, which was improved to 250 mg/L in the presence of the ShiA permease. The level of DHS accumulation can be reduced and the overall efficiency of the system improved by further optimization of the co-culture system.

### Example 6

### Co-Culture for the Production of 3-Aminobenzoate

One of the advantages of using the modular co-culture system is the ability to use the same first organism that produces an intermediate compound, but vary the second organism that is able to use the intermediate compound to produce a desired compound.

To engineer a modular co-culture system for the production of 3-aminobenzoate, the same first strain/module that secretes the DHS intermediate (P5.2) as described in Example 4 is used. The second strain/module is engineered to import DHS produced by the first cell by over-expressing ShiA and then convert DHS to 3-aminobenzoate through recombinant expression of pctV. The strains are co-cultured after which production of 3-aminobenzoate can be assessed. The level of DHS accumulation can be reduced and the overall efficiency of the system can be improved by further optimization of the co-culture system.

### Example 7

### Production of a Monoacetylated Dioxygenated Taxane by the Co-Culture System

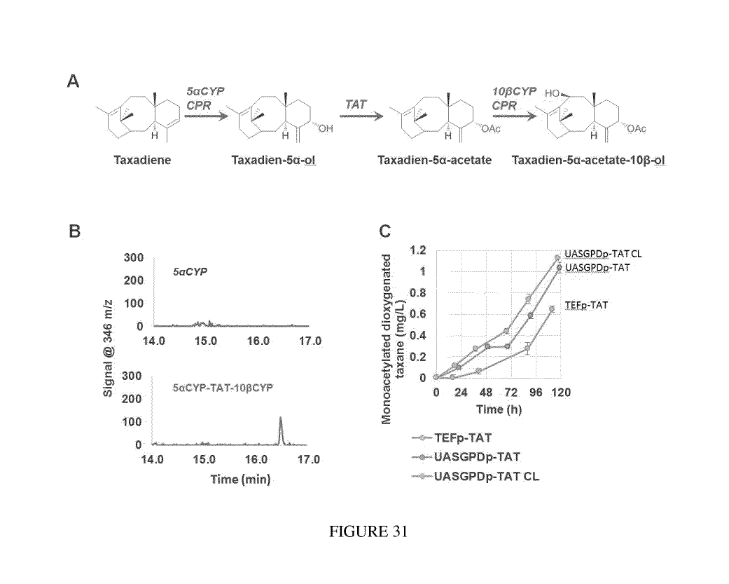

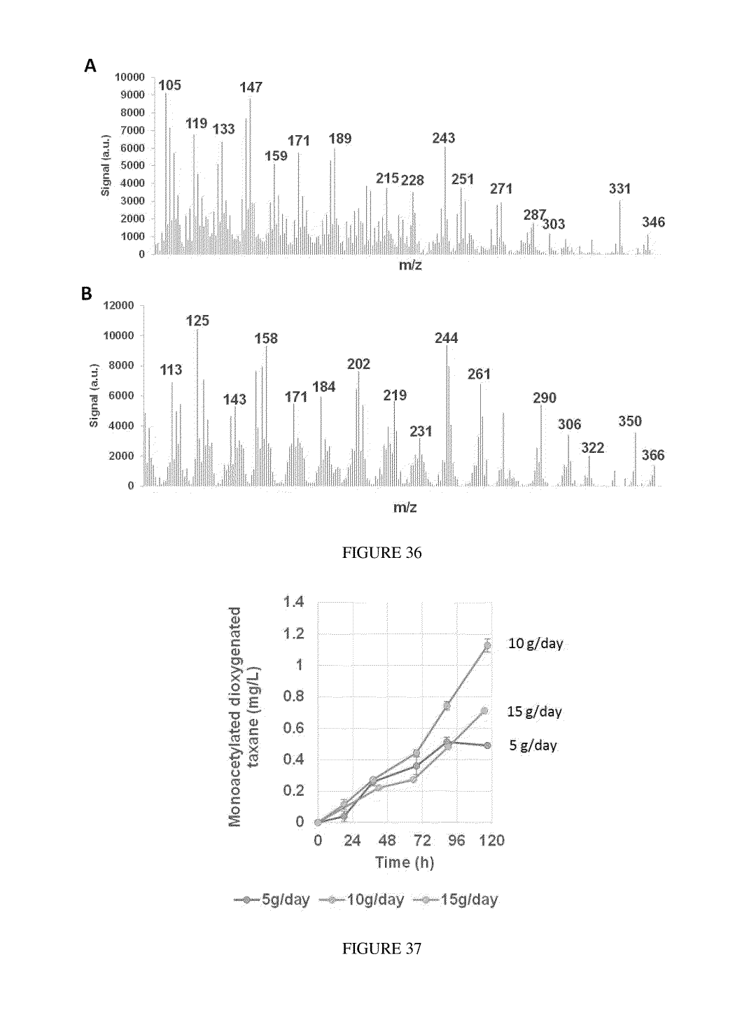

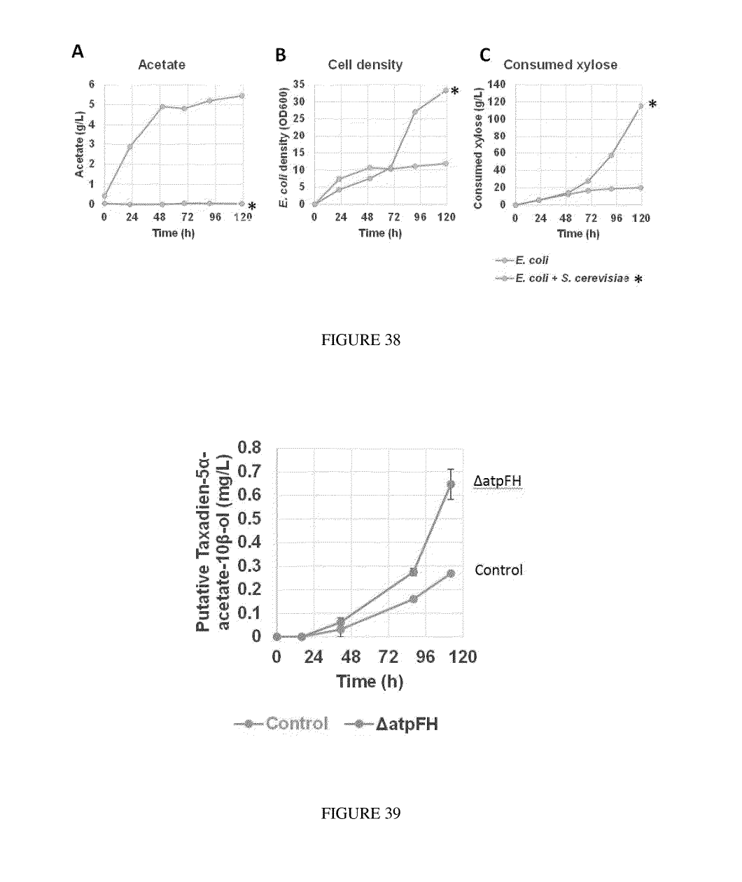

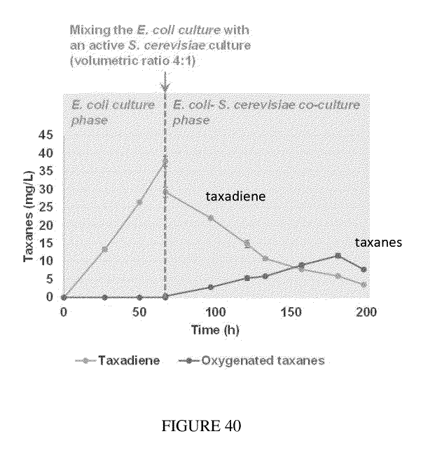

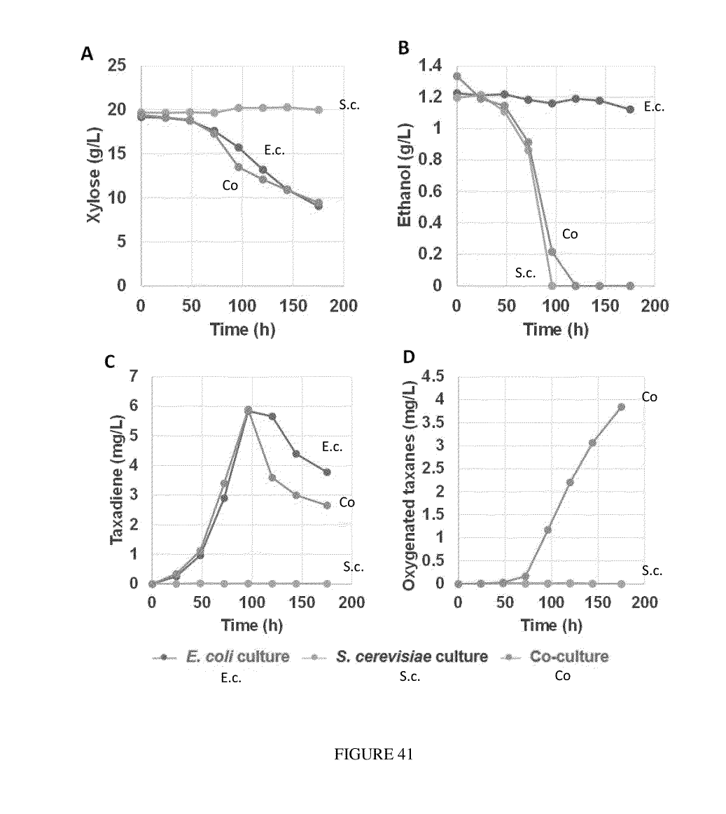

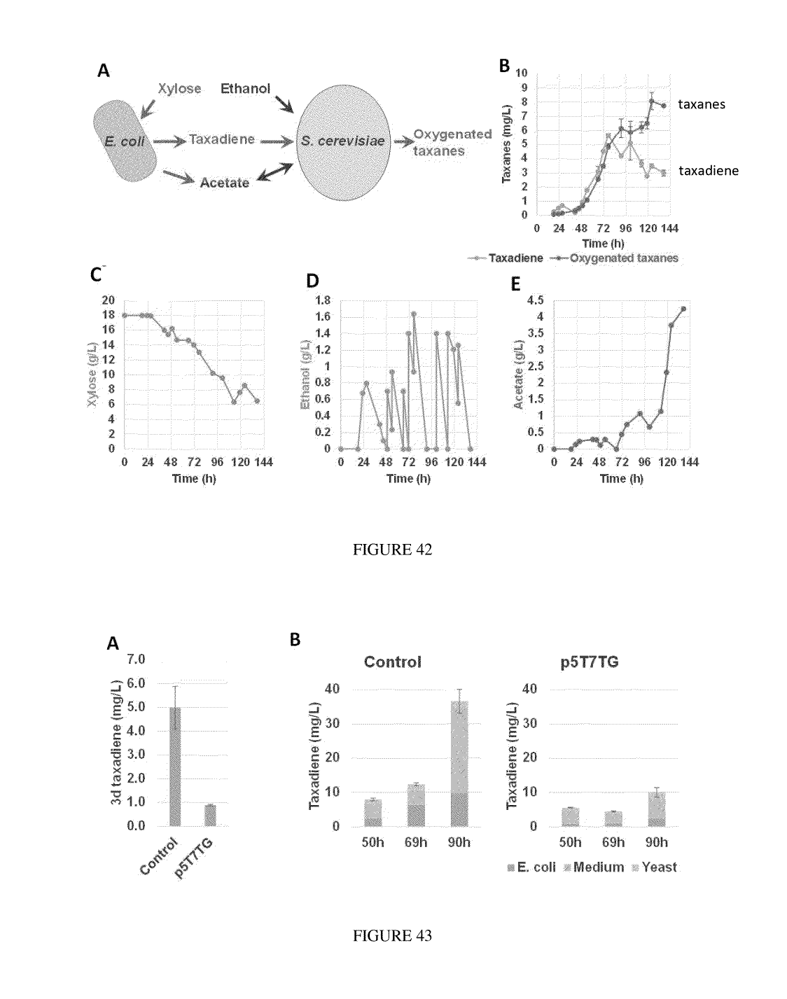

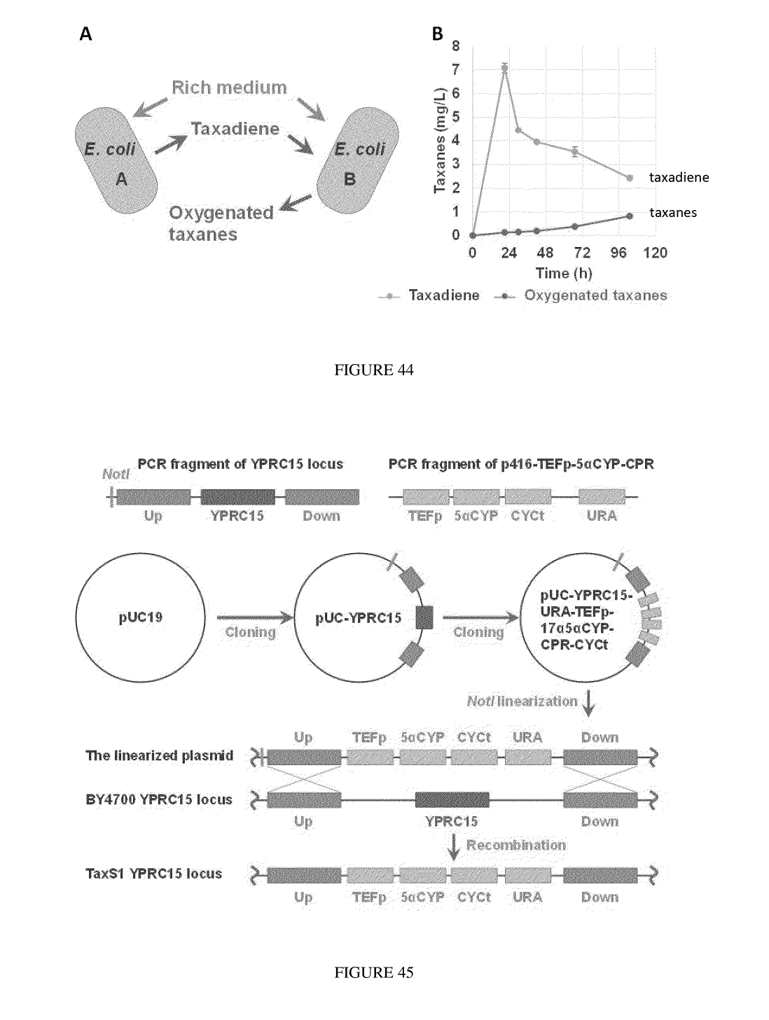

The co-culture system was further engineered to produce more advanced paclitaxel precursors. A prevailing theory of paclitaxel early-synthesis suggests taxadien-5α-ol to be acetylated at its C-5α position, followed by oxygenation at the C-10β position (FIG. 31A) (Guerra-Bubb et al., 2012). Because of the modular nature of the microbial consortium, such ability to functionalize taxadien-5α-ol could be conferred to the consortium by only modifying its yeast module. Taxadien-5α-ol acetyl-transferase (TAT) and taxane 10β-hydroxylase (10βCYP, fused with a CYP reductase) were co-expressed in yeast TaxS4 (Walker et al., 2007; Schoendorf et al., 2001; Ajikumar et al., 2010). When the resulting yeast (named as TaxS6) was co-cultured with E. coli TaxE4, the co-culture produced a monoacetylated dioxygenated taxane (molecular weight 346), which was identified as a single peak on the extracted ion chromatography (346 m/z, GCMS) and was absent in the control co-culture not expressing the TAT and 10βCYP (FIG. 31B). Subsequent ¹³C labeling experiments further confirmed that the monoacetylated deoxygenated taxane was indeed derived from taxadiene (FIG. 36). The identified compound could be taxadien-5α-acetate-10β-ol, an important intermediate in the paclitaxel synthesis because its spectrum contained many of its fragment ions (346, 303, 286, 271 and 243 m/z (FIG. 36)(Guerra-Bubb et al., 2012). To improve the titer and yield of this compound, we used a stronger promoter for expressing TAT (strain TaxS7), and the change of promoter indeed improved the titer from 0.6 mg/L to 1 mg/L (FIG. 31C), confirming the hypothesis that this step was limiting. We then operated the bioreactor under a xylose limiting condition, which further slightly increased the titer and also significantly improved the yield, by reducing the xylose consumption (from ˜120 g/L to 80 g/L, FIG. 31C, FIG. 37). This is the first report of producing a monoacetylated dioxygenated taxane from simple substrate (xylose) in microbes, which demonstrates the usefulness of the cellular consortium's modularity for synthesis of complex metabolites.

### Materials and Methods

E. coli Strains Used in the E. coli-S. cerevisiae Co-Culture

S. cerevisiae BY4719 (ATCC 200882, MATa trp1Δ463 ura3Δ0) was used to co-express 5αCYP-CPR, taxadien-5α-ol acetyl-transferase (TAT) and taxane 10β-hydroxylase with its reductase (10βCYP-CPR, as a fusion protein). Plasmid pUC-YPRC15-URA-GPDp-5αCYP-CPR-CYCt was linearized by using NotI and first transformed into BY4719 (YPRC15 locus), yielding yeast TaxS5. To further express TAT and 10βCYP-CPR in TaxS5, an integration vector (pUC-PDC6-TRP) was constructed that targeted locus PDC6 and contained TRP marker. First, plasmid pUC19 was combined with PCR fragment of BY4700 PDC6 locus via CLIVA (primer P46-P49 used), yielding integration plasmid pUC-PDC6. The auxotrophic marker (TRP) of plasmid p414-GPD was then cloned into pUC-PDC6 via CLIVA (primer P50-P53 used), yielding integration plasmid pUC-PDC6-TRP. After the construction of the integration vector, coding gene of Taxus cuspidata TAT was synthesized (Genscript) and cloned into plasmid pJA115 via CLIVA (primers P54-P57 were used), yielding p426-TEFp-TAT-ACTt (Avalos et al., 2013). Coding gene of Taxus cuspidata 10βCYP was synthesized (as gblocks gene fragments, Integrated DNA Technologies) and cloned into pUC-YPRC15-URA-GPDp-5αCYP-CPR to replace the 5αCYP via CLIVA (primers P58-P63 were used), yielding pUC-YPRC15-URA-GPDp-10βCYP-CPR-CYCt. The expression cassettes of these two plasmids (TEFp-TAT-ACTt and GPDp-10βCYP-CPR-CYCt) were assembled with part of the integration vector pUC-PDC6-TRP via CLIVA (primer P64-P69 used), yielding pUC-PDC6-TRP-(GPDp-10βCYP-CPR-CYCt)-(TEFp-TAT-ACTt). This plasmid was linearized by using NotI and transformed into TaxS5 (PDC6 locus), yielding yeast TaxS6 (Flagfeldt et al., 2009).

### Example 8

### Production of Other Oxygenated Isoprenoids by the Co-Culture

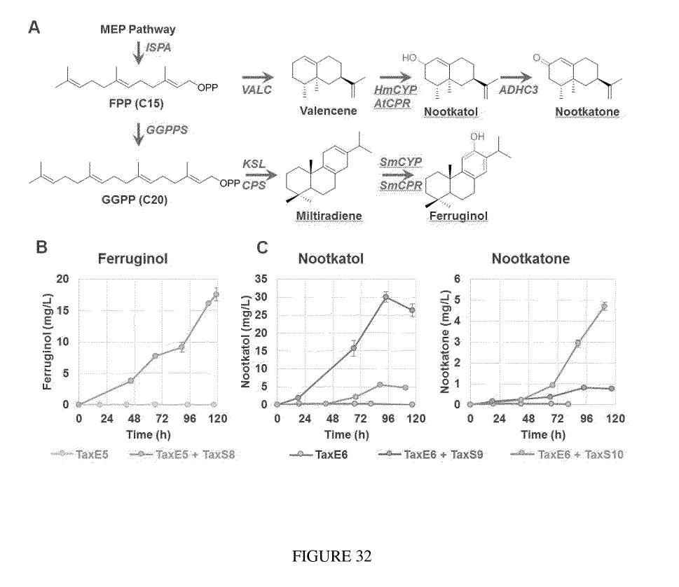

The cellular consortia described herein can be used for production of any metabolite if one of its precursors can cross cell membranes. Because the scaffold molecules for isoprenoids, the largest class of natural products, are generally membrane-permeable, the co-culture system should be applicable to synthesis of these molecules. To test this hypothesis, we examined the synthesis of another diterpene, ferruginol, the precursor of tanshinone, which is in clinical trial for treating heart disease (Zhou et al., 2012; Guo et al., 2013). The taxadiene synthase in E. coli TaxE4 was replaced with two enzymes (KSL and CPS, resulting in strain TaxE7) that are required for synthesizing miltiradiene, a membrane-crossing molecule (Zhou et al., 2012). S. cerevisiae BY4700 was also engineered to overexpress a specific CYP and its reductase (SmCYP and SmCPR, resulting in strain TaxS8), which were reported to oxygenate miltiradiene into ferruginol (FIG. 32A) (Guo et al., 2013. When E. coli TaxE5 and yeast TaxS8 were co-cultured in the medium containing xylose, the co-culture successfully produced 18 mg/L ferruginol (FIG. 32B), which exceeds the highest titer reported in the literature (10 mg/L by S. cerevisiae (Guo et al., 2013). This result not only supports that the co-culture strategy is generally applicable to diterpenes, but also demonstrates the advantages of co-culture over mono-culture systems, including the modular aspect in which one is able to construct parts of the pathway in parallel and achieve higher titer in virtue of cellular cooperation.

Finally, co-culture concept was applied to the synthesis of a sesquiterpene, nootkatone, which is a high-end fragrance molecule (Wriessnegger et al., 2014). The taxadiene synthase and geranylgeranyl diphosphate synthase in E. coli TaxE4 was replaced with a sesquiterpene synthase (VALC, resulting in strain TaxE8) to produce valencene, and in yeast S. cerevisiae BY4700 a specific CYP and its reductase (HmCYP and AtCPR, resulting in strain TaxS9) that can oxygenate valencene was expressed (FIG. 32A)(Wriessnegger et al., 2014). When these two cells were co-cultured, they produced 30 mg/L nootkatol and a small quantity of nootkatone (0.8 mg/L, FIG. 32C). Recently, a Pichia alcohol dehydrogenase (PpADH3C) was found to be able to oxidize nootkatol in its native host (Wriessnegger et al., 2014). This enzyme was introduced into yeast strain TaxS9, yielding strain TaxS10 which when co-cultured with E. coli TaxE8, increased the nootkatone titer by a factor of 5 (4 mg/L, FIG. 32C). Again, these results supported the hypothesis that the co-culture concept could be widely applicable to production of oxygenated isoprenoids.

### Materials and Methods

E. coli Strains Used in the E. coli-S. cerevisiae Co-Culture

To construct E. coli to produce miltiradiene, atpFH was knocked out of E. coli TaxE5, as described previously (primers P11 and P12 were used), resulting in strain TaxE6 (Causey et al., 2003). Then the plasmid p5T7-KSL-CPS-GGPPS was transformed into E. coli TaxE6, resulting in strain TaxE7. To obtain plasmid p5T7-KSL-CPS-GGPPS, KSL and CPS were amplified from synthetic DNA were assembled with part of p5T7TG1 via CLIVA (primer P13-P18 were used).

To construct E. coli to produce valencene, ISPA amplified from the E. coli genome, and VALC amplified from synthetic DNA were assembled with part of p5T7TG via CLIVA (primer P18-P23 were used), yielding plasmid p5T7-ISPA-VALC, which was transformed into E. coli TaxE6, resulting in strain TaxE8.

E. coli Strains Used in the E. coli-S. cerevisiae Co-Culture

To construct the yeast that can oxygenate miltiradiene, SmCYP and SmCPR amplified synthetic DNA were assembled with part of plasmid pUC-YPRC15-URA-UAS-GPDp-5αCYP-CPR-CYCt via CLIVA (primers P77-P82 were used), resulting in plasmid pUC-YPRC15-URA-UAS-GPDp-SmCYP-SmCPR-CYCt, which was transformed into S. cerevisiae BY4700, resulting in strain TaxS8. To construct the yeast that can produce nootkatone from valencene, HmCYP and AtCPR amplified from synthetic DNA were assembled with part of plasmid pUC-YPRC15-URA-UAS-GPDp-5αCYP-CPR-CYCt via CLIVA (primer P81-P86 used), resulting in plasmid pUC-YPRC15-URA-UAS-GPDp-HmCYP-AtCPR-CYCt, which was linearized by NotI and transformed into S. cerevisiae BY4700, resulting in strain TaxS9. To improve the nootkatone production, PpADHC3 amplified from Pichia pastoris genomic DNA was assembled with part of plasmid p426-TEFp-TAT-ACTt via CLIVA (primer), resulting in plasmid p426-TEFp-PpADHC3-ACTt; expression operon of this plasmid was further assembled with plasmid pUC-YPRC15-URA-UAS-GPDp-HmCYP-AtCPR-CYCt via CLIVA (primers P56, P57, P87 and P88 were used), resulting in plasmid pUC-YPRC15-URA-(UAS-GPDp-HmCYP-AtCPR-CYCt)-(TEFp-PpADHC3-ACTt), which was linearized by NotI and transformed into S. cerevisiae BY4700, resulting in strain TaxS10.

TABLE 2
Characterization of Yeast TaxS7 on two carbon sources

| | | Specific productivity of the |
|---|---|---|
| | Biomass yield (OD600/ | monoacetylated dioxygenated |
| | (g/L carbon source)) | taxane (μg/L/h/OD600) |
| Glucose | 2.35 ± 0.05 | 0.281 ± 0.037 |
| Acetate | 1.81 ± 0.08 | 0.190 ± 0.025 |

TABLE 3
Strains used in the studies

| Strain | Genotype |
|---|---|
| TaxE1 | MG1655_ΔrecA_ΔendA_DE3 ΔaraA::T7-MEP ΔlacY::T7-TG |
| TaxE2 | MG1655_ΔrecA_ΔendA_DE3 ΔaraA::T7-MEP ΔlacY::T7-TG |
| | p5trc-pta |
| TaxE3 | MG1655_ΔrecA_ΔendA_DE3 ΔaraA::T7-MEP ΔlacY::T7-TG |
| | p5trc-pta-trc-ackA |
| TaxE4 | MG1655_ΔrecA_ΔendA_DE3 ΔaraA::T7-MEP ΔlacY::T7-TG |
| | ΔatpFH::FRT-KanR-FRT |
| TaxE5 | MG1655_ΔrecA_ΔendA_DE3 ΔaraA::T7-MEP |
| TaxE6 | MG1655_ΔrecA_ΔendA_DE3 ΔaraA::T7-MEP ΔatpFH::FRT- |
| | KanR-FRT |
| TaxE7 | MG1655_ΔrecA_ΔendA_DE3 ΔaraA::T7-MEP ΔatpFH::FRT- |
| | KanR-FRT p5T7-KSL-CPS-GGPPS |
| TaxE8 | MG1655_ΔrecA_ΔendA_DE3 ΔaraA::T7-MEP ΔatpFH::FRT- |
| | KanR-FRT p5T7-ISPA-VALC |
| TaxE9 | MG1655_ΔrecA_ΔendA_DE3 p5trc-5αCYP-CPR |
| TaxE10 | MG1655_ΔrecA_ΔendA_DE3 p5T7TG |
| TaxE11 | MG1655_ΔrecA_ΔendA_DE3 ΔaraA::T7-MEP ΔlacY::T7-TG |
| | ΔatpFH::FRT-KanR-FRT p5T7TG |
| TaxS1 | MATa ura3Δ0::URA-TEFp-5αCYP-CPR-CYCt |
| TaxS2 | MATa ura3Δ0::URA-GPDp-5αCYP-CPR-CYCt |
| TaxS3 | MATa ura3Δ0::URA-ACSp-5αCYP-CPR-CYCt |
| TaxS4 | MATa ura3Δ0::URA-UAS-GPDp-5αCYP-CPR-CYCt |
| TaxS5 | MATa ura3Δ0::URA-GPDp-5αCYP-CPR-CYCt trp1Δ63 |
| TaxS6 | MATa ura3Δ0::URA-UAS-GPDp-5αCYP-CPR-CYCt |
| | trp1Δ63::TRP-(GPDp-10βCYP-CPR-CYCt)-(TEFp-TAT- |
| | ACTt) |
| TaxS7 | MATa ura3Δ0::URA-UAS-GPDp-5αCYP-CPR-CYCt |
| | trp1Δ63::TRP-(TEFp-10βCYP-CPR-ACTt-(UAS-GPDp- |
| | TAT-CYCt) |
| TaxS8 | MATa ura3Δ0::URA-UAS-GPDp-SmCYP-SmCPR-CYCt |
| TaxS9 | MATa ura3Δ0::URA-UAS-GPDp-HmCYP-AtCPR-CYCt |
| TaxS10 | MATa ura3Δ0::URA-(UAS-GPDp-HmCYP-AtCPR-CYCt)- |
| | (TEFp-PpADHC3-ACTt) |

TABLE 4
Primers used in the studies

| No. | Oligo | Sequence | |
|---|---|---|---|
| P1 | p5 LIC F | GTCGA*CCATC*ATCATCATC | SEQ ID NO: |
| | | | 9 |
| P2 | p5 LIC R | ATGTT*ATTCC*TCCTTATTT | SEQ ID NO: |
| | | | 10 |
| P3 | p5 - Ec.pta F | GGAAT*AACAT*GTGTCCCGTATTATT | SEQ ID NO: |
| | | | 11 |
| P4 | Ec.pta - p5 R | GATGG*TCGAC*TTACTGCTGCTGTGC | SEQ ID NO: |
| | | | 12 |
| P5 | p5 - Ec.ackA F | GGAAT*AACAT*ATGTCGAGTAAGTTA | SEQ ID NO: |
| | | | 13 |
| P6 | Ec.ackA - p5 R | GATGG*TCGAC*TCAGGCAGTCAGGCG | SEQ ID NO: |
| | | | 14 |
| P7 | p5 site 1 LIC F | ACTGG*GCCTT*GGCGTTTAAGGGCAC | SEQ ID NO: |
| | | | 15 |
| P8 | p5 site1 LIC R | GGAGT*CGCAT*TGGTGCTACGCCTGT | SEQ ID NO: |
| | | | 16 |
| P9 | p5 site1 - operon F | ATGCG*ACTCC*TGCATTAGG | SEQ ID NO: |
| | | | 17 |
| P10 | operon - p5 site1 R | AAGGC*CCAGT*CTTTCGACT | SEQ ID NO: |
| | | | 18 |
| P11 | atpF(KO)-FRT F | TAGTAAGCGTTGCTTTTATTTAAAGAGCAATAT | SEQ ID NO: |
| | | CAGAACGTTAACGGCTGGAGCTGCTTC | 19 |
| P12 | FRT2-atpH(KO) R | AGCGCGCCAGAGAGAAGCTCTGCCATTTGTTC | SEQ ID NO: |
| | | GTTTTTGGTTACCAATTAGCCATGGTCC | 20 |
| P13 | T7 RBS 1 - ksl F | TACCATGG*GCATGATG*AGCCTGGCATTTAAT | SEQ ID NO: |
| | | | 21 |
| P14 | ksl - GGGS_cps R | TTGCAGAA*CCACCACC*TTTACCGCGAACATT | SEQ ID NO: |
| | | | 22 |
| P15 | GGGS_cps F | GGTGGTGG*TTCTGCAA*GCCTGAGCAGC | SEQ ID NO: |
| | | | 23 |
| P16 | cps - tail of tds R | AGACCTGG*ATTGGATC*TTAGGCAACCGGCTC | SEQ ID NO: |
| | | | 24 |
| P17 | tail of tds F | GATCCAAT*CCAGGTCT*AA | SEQ ID NO: |
| | | | 25 |
| P18 | T7 RBS 1 LIC R | CATCATGC*CCATGGTA*TA | SEQ ID NO: |
| | | | 26 |
| P19 | T7 RBS 1 - valC_3-1 | TACCATGG*GCATGATG*GCCGAGATGTTCAAC | SEQ ID NO: |
| | F | GGC | 27 |
| P20 | ValC_3-1 - GS.LK | GATCCGG*TGCTGCC*GGGGATGATGGGCTCGA | SEQ ID NO: |
| | | C | 28 |
| P21 | GS.LK_Ec.ispA F | GGCAGCA*CCGGATC*CGACTTTCCGCAGCAA | SEQ ID NO: |
| | | | 29 |
| P22 | Ec. ispA - tail of | AGTTTTGA*CGAAAGGC*TTATTTATTACGCTG | SEQ ID NO: |
| | ggpps R | GATG | 30 |
| P23 | tail of ggpps F | GCCTTTCG*TCAAAACT*AA | SEQ ID NO: |
| | | | 31 |
| P24 | XbaI-bovinel7a F | GCtctagaAAAATGGCTCTGTTATTAGCAGTT | SEQ ID NO: |
| | | | 1 |
| P25 | CPR-his-HindIII R | GCaagcttTTAgtgatggtgatgatgatgCCAAA | SEQ ID NO: |
| | | TATCCCGTAAGTAGC | 2 |
| P26 | YPRC-p4xx F | TCGCAAA*ACTAAAG*GGAACAAAAGCTG | SEQ ID NO: |
| | | | 32 |
| P27 | p4xx-YPRC R | TTCCATT*ATCAGAG*CAGATTGTACTGAGA | SEQ ID NO: |
| | | | 33 |
| P28 | p4xx-YPRC F | CTCTGAT*AATGGAA*GGTCGGGATGA | SEQ ID NO: |
| | | | 34 |
| P29 | YPRC-p4xx R | CTTTAGT*TTTGCGA*AACCCTATGCT | SEQ ID NO: |
| | | | 35 |
| P30 | NotI-YPRC15 F | GCgcggccgcTTTATATCATATAATTAAGACACAA | SEQ ID NO: |
| | | AAG | 36 |
| P31 | YPRC15-EcoRI R | GCgaattcATAAAGCAGCCGCTACCA | SEQ ID NO: |
| | | | 37 |
| P32 | EcoRI-pUC19 F | GCgaattcAAAGCCTGGGGTGCCTAA | SEQ ID NO: |
| | | | 38 |
| P33 | pUC19-NotI R | GCgcggccgcAGGTGGCACTTTTCGGGG | SEQ ID NO: |
| | | | 39 |
| P34 | GPDp-17a F | AACAAA*ATGGCT*CTGTTATTAGCAG | SEQ ID NO: |
| | | | 40 |
| P35 | p4xx-GPDp R | TTTTTTAT*CAGCTT*TTGTTCCCTTT | SEQ ID NO: |
| | | | 41 |
| P36 | p4xx-GPDp F | AAGCTG*ATAAAAAA*CACGCTTTTTC | SEQ ID NO: |
| | | | 42 |
| P37 | GPDp-17a R | AGCCAT*TTTGTT*TGTTTATGTGTGT | SEQ ID NO: |
| | | | 43 |
| P38 | ACS1p-17a F | TGTGCT*ATGGCT*CTGTTATTAGCAG | SEQ ID NO: |
| | | | 44 |
| P39 | p4xx-ACS1p R | ATTGTT*CAGCTT*TTGTTCCCTTTAG | SEQ ID NO: |
| | | | 45 |
| P40 | p4xx-ACS1p F | AAGCTG*AACAAT*CTGTTTATTACCC | SEQ ID NO: |
| | | | 46 |
| P41 | ACS1p-17a | AGCCAT*AGCACA*GTGGGCAATG | SEQ ID NO: |
| | | | 47 |
| P42 | p4xx - UAS(TEF) F | ACAAA*AGCTG*AATGTTTCTACTCCT | SEQ ID NO: |
| | | | 48 |
| P43 | UAS(CLB) - GPD R | TGTTT*TTTAT*GGGACAGGCACCGAA | SEQ ID NO: |
| | | | 49 |
| P44 | GPD LIC F | ATAAA*AAACA*CGCTTTTTC | SEQ ID NO: |
| | | | 50 |
| P45 | p4xx (Seq-F) R | CAGCT*TTTGT*TCCCTTTAG | SEQ ID NO: |
| | | | 51 |
| P46 | pUC-PDC6 F | GCGG*CCGC*CTTT*CAAG*GGTGGGGG | SEQ ID NO: |
| | | | 52 |
| P47 | pDC6-pUC R | CAGG*CTTT*GGCT*GAAC*AACAGTCTCTCC | SEQ ID NO: |
| | | | 53 |
| P48 | pDC6-pUC F | GTTC*AGCC*AAAG*CCTG*GGGTGCCT | SEQ ID NO: |
| | | | 54 |
| P49 | pUC-PDC6 R | CTTG*AAAG*GCGG*CCGC*AGGTGGCA | SEQ ID NO: |
| | | | 55 |
| P50 | Inter - Express F | TAAAGGGA*ACAAAAGC*TG | SEQ ID NO: |
| | | | 56 |
| P51 | Marker - Inter R | GCAGATTG*TACTGAGA*GT | SEQ ID NO: |
| | | | 57 |
| P52 | Marker - Inter F | TCTCAGTA*CAATCTGC*TC | SEQ ID NO: |
| | | | 58 |
| P53 | Inter - Express R | GCTTTTGT*TCCCTTTA*GT | SEQ ID NO: |
| | | | 59 |
| P54 | p4xx RE - TAT F | TAGAA*CTATG*GAAAAAACTGATCTG | SEQ ID NO: |
| | | | 60 |
| P55 | TAT - Myc LIC R | TCAAT*TTTTG*AACTTTGGCCACGTA | SEQ ID NO: |
| | | | 61 |
| P56 | Myc LIC F | CAAAA*ATTGA*TTTCTGAAG | SEQ ID NO: |
| | | | 62 |
| P57 | p4xx RE LIC R | CATAG*TTCTA*GAGCTAGC | SEQ ID NO: |
| | | | 63 |
| P58 | Linker CPR LIC F | GGCAG*CACCG*GATCC | SEQ ID NO: |
| | | | 64 |
| P59 | GPD LIC R | CATTT*TGTTT*GTTTATGTG | SEQ ID NO: |
| | | | 65 |
| P60 | GPD - CO.10bCYP F | AAACA*AAATG*GACTCCTTCATCTTC | SEQ ID NO: |
| | | | 66 |
| P61 | Co.10bCYP part1 - | GACAAGAT*TTCGTCCA*ACTTCAATC | SEQ ID NO: |
| | part2 R | | 67 |
| P62 | Co.10bCYP part1 - | TGGACGAA*ATCTTGTC*CTCCTTGAT | SEQ ID NO: |
| | part2 F | | 68 |
| P63 | CO.10bCYP - Linker | CGGTG*CTGCC*AGATCTTGGGAACAA | SEQ ID NO: |
| | CPR R | | 69 |
| P64 | Inter - GPDp F | AAAGCT*AGTTTATC*ATTATCAATAC | SEQ ID NO: |
| | | | 70 |
| P65 | CYC1t - ACT1t rc R | TATCATAT*CAAATTAA*AGCCTTCGA | SEQ ID NO: |
| | | | 71 |
| P66 | CYC1t - ACT1t rc F | TTAATTTG*ATATGATA*CACGGTCCA | SEQ ID NO: |
| | | | 72 |
| P67 | TEFp rc - Inter R | ATCGGC*ATAGCTTC*AAAATGTTTCT | SEQ ID NO: |
| | | | 73 |
| P68 | TEFp rc - Inter F | GAAGCTAT*GCCGAT*TTCGGCCTATT | SEQ ID NO: |
| | | | 74 |
| P69 | Inter - GPDp | GATAAACT*AGCTTT*TGTTCCCTTT | SEQ ID NO: |
| | | | 75 |
| P70 | GPD - TAT F | AAACA*AAATG*GAAAAAACTGATCTG | SEQ ID NO: |
| | | | 76 |
| P71 | TAT - CYCt R | TAAGC*TTTTA*AACTTTGGCCACGTA | SEQ ID NO: |
| | | | 77 |
| P72 | CYCt LIC F | TAAAA*GCTTA*TCGAT | SEQ ID NO: |
| | | | 78 |
| P73 | p4xx RE - Co.10CYP | TAGAA*CTATG*GACTCCTTCATCTTC | SEQ ID NO: |
| | F | | 79 |
| P74 | CPR - Myc LIC R | TCAAT*TTTTG*CCAAATATCCCGTAA | SEQ ID NO: |
| | | | 80 |
| P75 | Inter - UAS (45-F) | CAAAAGCT*AATGTTTC*TACTCCTTT | SEQ ID NO: |
| | | | 81 |
| P76 | Inter - UAS (45-R) | GAAACATT*AGCTTTTG*TTCCCTTTA | SEQ ID NO: |
| | | | 82 |
| P77 | GPD - Sm.CYP F | ATAAACAA*ACAAAATG*GACTCTTTTCCATTA | SEQ ID NO: |
| | | TTG | 83 |
| P78 | SmCYP - LK R | GATCCGG*TGCTGCC*GGACTTGACGATTGG | SEQ ID NO: |
| | | | 84 |
| P79 | LK_SmCPR1 F | GGCAGCA*CCGGATC*CGAACCATCCTCTAAAA | SEQ ID NO: |
| | | A | 85 |
| P80 | Sm. CPR - His R | GATGGTGA*TGATGATG*CCAGACATCTCTCAA | SEQ ID NO: |
| | | GTA | 86 |
| P81 | 6xHis LIC F | CATCATCA*TCACCATC*AC | SEQ ID NO: |
| | | | 87 |
| P82 | GPD LIC R2 | CATTTTGT*TTGTTTAT*GTG | SEQ ID NO: |
| | | | 88 |
| P83 | GPD - Hm.CYP F | ATAAACAA*ACAAAATG*CAATTCTTCTCCTTG | SEQ ID NO: |
| | | G | 89 |
| P84 | Hm.CYP - LK R2 | GATCCGG*TGCTGCC*TTCTCTGGATGGTTG | SEQ ID NO: |
| | | | 90 |
| P85 | LK_At.CPR F | GGCAGCA*CCGGATC*CACTTCTGCCTTGTAT | SEQ ID NO: |
| | | | 91 |
| P86 | At. CPR - His R | GATGGTGA*TGATGATG*CCAGACATCTCTCAA | SEQ ID NO: |
| | | | 92 |
| P87 | p4xx RE - | TAGAA*CTATG*ACCACAGTTTTCGCT | SEQ ID NO: |
| | Pp.ADH_C3 F | | 93 |
| P88 | Pp.ADH_C3 - Myc | TCAAT*TTTTG*CCCCCTGACTTTACT | SEQ ID NO: |
| | LIC R | | 94 |

*indicates a phosphorothioate bond

TABLE 5
Sequences of synthetic genes used in the studies

| Synthetic gene | Sequence |
|---|---|
| UAS_{TEF}-UAS_{CIT1}- | aatgtttctactccttttttactcttccagattttctcggactccgcgcatcgccgtaccacttcaaaacacccaag |
| UAS_{CLB2} | cacagcatactaaatttcccctctttcttcctctagggtgtcgttaattacccgtactaaaggtttggaaaagaaaa |
| (SEQ ID NO: 95) | aagagaccgcctcgtttctttttcttcgtcgaaaaaggcaataaaaattttttagagattactacatattccaacaa |
| | gaccttcgcaggaaagtatacctaaactaattaaagaaatctccgaagttcgcatttcattgaacggctcaatta |
| | atctttgtaaatatgagcgtttttacgttcacattgcctttttttttatgtatttaccttgcatttttgtgctaaaag |
| | gcgtcacgtttttttccgccgcagccgcccggaaatgaaaagtatgacccccgctagaccaaaaatacttttgtgttat |
| | tggaggatcgcaatccctttcagtggaattattagaatgaccactactccttctaatcaaacacgcggaaatagc |
| | cgccaaaagacagattttattccaaatgcgggtaactatttgtataatatgtttacatattgagcccgtttaggaaa |
| | gtgcaagttcaaggcactaatcaaaaaaggagatttgtaaatatagcgaccgaatcaggaaaaggtcaacaa |
| | cgaagttcgcgatatggatgaacttcggtgcctgtccc |
| TAT | atggaaaaaactgatctgcatgtcaatctgattgaaaaagttatggttggtcctagccctccactgccgaaaact |
| (SEQ ID NO: 96) | accctgcaactgtccagcattgataacctgccgggtgtacgcggtagcattttcaacgcactgctgatctataa |
| | cgcaagcccgagcccgaccatgatctctgcagatccagcaaaaccgatccgcgaagcgctggcgaaaatt |
| | ctggtttactacccgccatttgcaggccgcctgcgtgaaaccgaaaacggtgatctggaagttgagtgcaccg |
| | gtgaaggtgcgatgttcctggaagcgatggcggacaacgaactgagcgttctgggtgactttgacgattcca |
| | acccgtcttttcagcaactgctgttttccctgcctctggataccaactttaaagatctgtccctgctggttgttcag |
| | gtgacccgctttacctgtggcggtttcgtcgtcggcgttagcttccaccacggcgtttgcgacggccgtggcg |
| | ccgcacagtttctgaaaggcctggctgaaatggcacgtggtgaggtgaagctgtctctggaaccgatttggaa |
| | ccgcgaactggttaaactggatgacccgaaatacctgcagttctttcacttcgagttcctgcgtgcgccgagca |
| | tcgtagagaaaatcgttcagacctatttcatcattgatttcgagacgatcaactatatcaaacagtctgtaatgga |
| | agagtgcaaggagactgctcaccacgaagtggcaccgctatgacttggatcgcgcgtactcgtgcgacca |
| | gattccggaaagcgaatacgtgaagatcctgacggcatggacatgcgtaactcatcaacccgccgctgccg |
| | tccggctattacggtaacagcatcggcacggcgtgtgctgtggacaacgttcaggatctgctgtctggttctct |
| | gctgcgtgctatcatgattatcaagaaatccaaagtctccctgaacgacaatttcaaatctcgcgcggtggtaa |
| | aaccgtccgaactggacgtgaacatgaatcacgaaaacgtagtcgccacgctgactggtcccgtctgggttt |
| | cgacgaagtagacttcggctggggtaatgctgtatctgtttctccggtgcagcagcagtctgctctggccatgc |
| | aaaactacttcctgttcctgaaaccgtctaaaaacaaaccagatggtatcaaaatcctgatgatctgccgctgtc |
| | taagatgaaatctttcaaaatcgaaatggaagccatgatgaagaaatacgtggccaaagtttaa |
| 10Bcyp | atggactccttcatcttcttgagatccattggtactaagttcggtcaattggaatcttccccagctattttgtctttga |
| (SEQ ID NO: 97) | ctttggctccaattttggccatcatcttgttgttgttattcagatacaaccacagatcctctgttaagttgccaccag |
| | gtaaattgggttttccattgattggtgaaaccatccaattattgagaaccttgagatctgaaaccccacaaaagtt |
| | cttcgatgacagattgaaaaagtttggtccagtctacatgacctcattgataggtcatccaactgttgttttgtgtg |
| | gtccagctggtaacaaattggattgtctaacgaagataagaggagaaatggaaggtccaaagtcatcatga |
| | agttgatcggtgaagattctatcgagctaagagaggtgaagatcacagaattagagaactgctaggctagatt |
| | tagggtgctcaagattacaaaactacttgggtagaatgtcctccgaaattggtcatcatataacgaaaagtgg |
| | aagggtaaggatgaagttaaggattgccattggtcagaggatgattactctattgatccaccttgacttcgac |
| | gttaatgatggtcatcaacaaaagcaattgcaccacttgaggaaaccattaggaggactagtccgaccattg |
| | gattaccaggtactagatacagaaagggatacaagctagattgaagttggacgaaatcagtcctccttgatta |
| | agcgtagaagaagagatttgagatccggtattgcaccgatgatcaagatttgagtctgtcttgagaccttcag |
| | agatgaaaagggtaactctttgaccgatcaaggtatcttggataacttctctgctatgttccatgatcttacgata |
| | caactgttgctccaatggctttgatcttcaagttgttgtactctaacccagaataccacgaaaaggttttccaaga |
| | acaattggaaatcatcggtaacaagaaagaaggtgaagaaatctcctggaaggacttgaaatctatgaagtac |
| | acttggcaagccgtccaagaatattgagaatgtatccaccagattcggtattacagaaaggccattaccgata |
| | tccattacgatggttacactattccaaagggaggagagattgtgactccatataccactcacttgagagaaga |
| | atactttccagaaccagaagaattcagaccatccagatttgaagatgaaggtagacatgttactccatacactta |
| | cgttccatttggtggtggatgagaacttgtccaggagggaattttctaagatcgaaatcttgagttcgtccacc |
| | acttcgttaagaacttctcatcttacattccagtcgacccaaacgaaaaagttttgtctgatccattgccaccatta |
| | ccagctaatggtttctctattaagttgttcccaagatcttaa |
| KSL | atgagcctggcatttaatccggcagcaaccgcatttagcggtaatggtgcacgtagccgtcgtgaaaactttcc |
| (SEQ ID NO: 98) | ggttaaacatgttaccgttcgtggttttccgatgattaccaataaaagcagctttgccgttaaatgcaatctgacc |
| | accaccgatctgatgggcaaaattgcagaaaaattcaaaggcgaggatagcaattttccggcagccgcagc |
| | agttcagcctgcagcagatatgccgagcaatctgtgtattattgataccctgcagcgtctgggtgttgatcgttat |
| | tttcgtagcgaaattgataccatcctggaagatacctatcgtctgtggcagcgtaaagaacgtgcaatttttagc |
| | gataccgcaattcatgcaatggcatttcgtctgctgcgtgttaaaggttatgaagttagcagcgaagaactggc |
| | accgtatgcagatcaagaacatgtggatctgcagaccattgaagttgcaaccgttattgaactgtatcgtgcag |
| | cacaagaacgtaccggtgaagatgaaagcagcctgaaaaaactgcatgcatggaccaccacatttctgaaa |
| | cagaaactgctgaccaatagcatcccggataaaaaactgcacaaactggtggaatactatctgaaaaactttc |
| | acggcattctggatcgtatgggtgacgtcagaatctggatctgtacgatattagttattaccgtaccagcaaag |
| | cagccaatcgattagtaatctgtgctccgaagattactggcatttgcacgtcaggatataacatagtcaggca |
| | cagcatcagaaagaactgcagcagctgcaacgttggtatgccgattgtaaactggataccctgaaatatggtc |
| | gtgatgttgttcgtgttgcaaattttctgaccagcgcaattattggtgatccggaactgagtgatgttcgtattgat |
| | ttgcacagcatattgactggtgacccgcatcgatgatttttttgatcatcgtggtagccgtgaagagagctacaa |
| | aattctggaactgatcaaagaatggaaagaaaaaccggcagcagaatatggtagcgaagaagttgaaattct |
| | gttcaccgcagtgtataataccgtgaatgaactggcagaacgtgcccatgttgaacagggtcgtagcgttaaa |
| | gatttcctgattaaactgtgggtgcagatcctgagcatctttaaacgtgagctggatacctggtcagatgatacc |
| | gcactgaccctggatgattatctgagcgcaagctgggttagcattggagtcgtatagtattctgatgtccatgc |
| | agttcattggcatcaaactgtcagatgaaatgctgctgagcgaagaatgtattgatctgtgtcgtcatgttagcat |
| | ggtggatcgcctgctgaatgatgacagaccatgaaaaagaacgcaaagagaataccggtaatagcgttacc |
| | ctgctgctggcagcaaataaagatgatagcagttttaccgaagaagaggcaattcgtattgcaaaagaaatgg |
| | ccgaatgtaatcgtcgtcagctgatgcagattgtgtataaaaccggtacaattatccgcgtcagtgcaaagata |
| | tgtttctgaaagtagccgcattgggtgttatctgtatgcaagcggtgatgaatttaccagtccgcagcagatgat |
| | ggaagatatgaaaagcctggtttatgaaccgctgaccattcatccgctggagcaaataatgacgcggtaaat |
| | aa |
| CPS | atggcaagcctgagcagcaccattctgagccgtagtccggcagcacgtcgtcgtattacaccggcaagcgc |
| (SEQ ID NO: 99) | aaaactgcatcgtccggaatgttttgcaaccagcgcatggatgggtagcagcagcaaaaatctgagcctgag |
| | ctatcagctgaaccacaaaaaaatcagcgagcaaccgttgatgcaccgcaggttcatgatcacgatggcacc |
| | accgttcatcagggtcatgatgcagttaaaaacattgaagatccgatcgaatatatccgtaccctgctgcgtac |
| | caccggtgatggtcgtattagcgttagcccgtatgataccgcatgggttgcaatgattaaagatgttgaaggtc |
| | gtgatggtccgcagtttccgagcagcctggaatggattgttcagaatcagctggaagatggtagctggggtg |
| | atcagaaactgttttgtgtttatgatcgtctggtgaataccattgcatgtgttgttgcactgcgtagctggaatgttc |
| | atgcacataaagttaaacgtggcgtgacctatatcaaagaaaacgtggataaactgatggaaggcaacgaag |
| | aacatatgacctgtggttttgaagttgtttttccggcactgctgcagaaagcaaaaagcctgggtatcgaagatc |
| | tgccgtatgattcaccggcagttcaagaggatatcatgacgtgaacaaaaactgaaacgcattccgctggaa |
| | atcatgcataaaattccgaccagtctgctgatagcctggaaggtctggaaaatctggattgggacaaactgct |
| | gaaactgcagagcgcagatggtagttttctgaccagcccgagcagtaccgcatttgcatttatgcagaccaaa |
| | gatgagaaatgctatcagttcatcaaaaacaccatcgacaccataatggtggtgcaccgcatacctatccggtt |
| | gatgtttttggtcgtctgtgggcaattgatcgcctgcagcgtctgggtattagccgtttttttgaaccggaaattgc |
| | agattgtctgagccacattcacaaattctggaccgataaaggtgtttttagcggtcgtgaaagcgaattttgcga |
| | tattgatgataccagtatgggtatgcgtctgatgcgtatgcatggttatgatgagatccgaatgttctgcgcaact |
| | ttaaacagaaagatggcaaatttagctgctatggtggtcagatgattgaaagcccgagcccgatttataacctg |
| | tatcgtgcaagccagctgcgttttccgggtgaagaaattctggaagatgccaaacgttttgcctatgatttcctg |
| | aaagaaaaactggccaataaccagatcctggataaatgggttattagcaaacatctgccggatgaaattaaac |
| | tgggcctggaaatgccgtggctggcaaccctgcctcgtgttgaagcaaaatactatattcagtattatgccggt |
| | agcggtgatgtgtggattggtaaaaccctgtatcgcatgccggaaattagcaatgatacctatcatgatctggc |
| | caaaaccgattttaaacgttgtcaggcaaaacaccagtttgagtggctgtatatgcaagaatggtatgaaagct |
| | gcggcattgaagaatttggcattagccgtaaagatctgctgctgagctattttctggcaaccgcaagcatctttg |
| | aactggaacgtaccaatgaacgtattgcatgggccaaaagccagattattgcaaaaatgatcaccagctttttta |
| | acaaagaaaccacgagcgaagaagataaacgcgcactgctgaatgaactgggtaacattaatggtctgaat |
| | gataccaatggtgcaggtcgtgaaggtggtgcgggtagcattgcactggccaccctgacccagtactggaa |
| | ggttttgatcgttatacccgtcaccagctgaaaaatgcatggtcagtttggctgacccagctgcagcatggtga |
| | agcagatgatgcagaactgctgaccaataccctgaatatttgtgccggtcatattgcctttcgcgaagaaatcct |
| | ggcacacaatgaatataaagcactgagcaatctgacgagcaaaatagtcgccagctgagattattcagagc |
| | gaaaaagaaatgggtgtggaaggtgaaattgccgcaaaaagcagcattaaaaacaaagaactggaagagg |
| | atatgcagatgctggttaaactggactggagaaatatggtggtattgaccgcaatatcaaaaaagcatactgg |
| | cagttgccaaaacctattattaccgtgcatatcatgcagccgataccattgatacccatatgttcaaagttctgttt |
| | gagccggttgcctaa |
| SmCYP | atggactcattccattattggctgccttgtttttcattgctgctactattaccacttgtccaccgtagaagaagaaa |
| (SEQ ID NO: 100) | tttgccaccaggtccatttccatatccaatcgttggtaatatgttgcaattgggtgctaacccacatcaagtttttgc |
| | taagagtctaagagatacggtccattgatgtccattcatagggttccagtacaccgttatagtctatcaccaga |
| | aatggccaaagaaatcttgcatagacatggtcaagattctccggtagaactattgctcaagctgacatgcttgt |
| | gatcacgataagatttctatgggttttttgccagttgcctctgaatggagagatatgagaaagatctgcaaagaa |
| | caaatgttctccaatcaatccatggaagcttctcaaggtttgagaagacaaaagttgcaacaattattggaccac |
| | gtccaaaagtgttctgattctggtagagctgagatattagagaagctgattcattaccaccttgaatttgatgtct |
| | gctaccttgattatcacaagctaccgaatttgattccaaggctaccatggaattcaaagaaattattgaaggtgt |
| | tgccaccatcgaggtgaccaaattagctgattacttcccaatcttaagaccattcgatccacaaggtgttaaga |
| | gaagagctgatgtttttttcggtaagttgttggccaagatcgaaggttatttgaacgaaagattggaatccaaga |
| | gagctaatccaaacgctccaaagaaggatgatttcaggaaatcgagtcgatatcatccaagccaacgaattc |
| | aagttgaaaacccatcatttcacccacttgatgaggatttgatgaggtggactgataccaacaccacactatt |
| | gaatgggctatgtctgaattggttatgaacccagataagatggctagattgaaggctgaattgaaatctgagct |
| | ggtgacgaaaagatcgttgatgaatctgctatgccaaagttgccatacttgcaagctgttatcaaagaagtcat |
| | gagaattcatccacctggtcattgagttaccaagaaaagctgaatccgatcaagaagtcaacggttacttaatt |
| | ccaaagggtactcaaatcttgattaacgcttacgccattggtagagatccatctatttggactgatccagaaactt |
| | ttgacccagaaagattcttggacaacaagatcgatttcaagggtcaagactacgaattattgccatttggttcag |
| | gtagaagagtttgtccaggtatgccattggctactagaatattgcatatggctactgctactttggttcacaatttc |
| | gattggaagttggaagatgattctactgctgctgctgatcatgctggtgaattatttggtgttgctgttagaagag |
| | cagtcccattgagaattattccaatcgtcaagtcctaa |
| SmCPR | atggaaccatcctctaaaaagttgtccccattggatttcattaccgccattttgaagggtgatattgaaggtgttg |
| (SEQ ID NO: 101) | ctccaagaggtgttgcagctatgttgatggaaaacagagatttggctatggttttgactacctctgttgctgttttg |
| | attggttgcgttgttgttttggcttggagaagaactgctggttctgctggtaaaaaacaattgcaaccaccaaagt |
| | tggttgttccaaaaccagctgctgaacctgaagaagctgaagacgaaaaaactaaggtcagtgttttcttcggt |
| | actcaaactggtactgctgaaggattgctaaagcttttgccgaagaagctaaagctagatatccacaagctaa |
| | gttcaaggttatcgatttggatgattacgctgccgatgatgatgaatacgaagaaaagttgaagaaagaatcctt |
| | ggccttcttcttcttggcttcttatggtgatggtgaacctactgataatgctgctagattttacaagtggttcaccga |
| | aggtaaggatagagaagattggttgaagaacttgcaatacggtgtttttggtttgggtaacagacaatacgaac |
| | acttcaacaagattgccatcgttgtcgatgatttgattactgaacaaggtggtaagaagttggttccagttggttta |
| | ggtgatgatgatcaatgcatcgaagatgatttttccgcttggagagaattggtttggccagaattggataagttgt |
| | tgagaaatgaagatgatgctactgttgctactccatataccgctgttgttttacaatacagagttgtcttgcacgat |
| | caaactgatggtttgatcacagaaaatggttctccaaatggtcatgctaacggtaacactatctatgatgctcaa |
| | catccatgtagagctaacgttgctgttagaagagaattgcatactccagcttcagatagatcttgtacccatttgg |
| | aattcgatacttcaggtactggtttggtttacgaaactggtgatcatgttggtgtttactgcgaaaacttgttggaa |
| | aatgtcgaagaagccgaaaagttattgaacttgtctccacaaacctacttctccgttcatactgataacgaagat |
| | ggtactccattgtctggttcttcattgccaccaccatttccaccatgtactttgagaactgctttgactaagtacgc |
| | cgatttgatttctatgccaaagaagtctgttttggttgctttggctgaatacgcctctaatcaatcagaagctgata |
| | gattgagatacttggcttcaccagatggtaaagaagaatacgcccaatatatcgttgcctcccaaagatcattat |
| | tggaagttatggctgaattcccatctgctaaaccaccattgggtgttttttttgctgctattgctcctagattgcaac |
| | ctagattctactccatttcttcctccccaaaaattgctccaactagagttcatgttacctgtgctttggtttatgataa |
| | gactccaactggtagaatccataagggtatttgttctacctggattaagaacgctgttccattggaagaatcttca |
| | gattgctcttgggctccaattttcatcagaaactctaactttaagttgccagccgatccaaaggttccaattatcat |
| | ggttggtccaggtacaggtttagctccttttagaggtttcttacaagaaagattggccttgaaagaatctggtgct |
| | gaattgggtccagctattttgttttttggttgtagaaacagaaagatggacttcatatacgaagatgaattgaactc |
| | cttcgttaaggttggtgccatttctgaattgatcgttgctttttctagagaaggtccagccaaagaatacgttcaac |
| | ataagatgtctcaaagagcctccgatatttggaagatgatatctgatggtggttacatgtacgtttgtggtgatgc |
| | taaaggtatggctagagatgttcatagaaccttgcataccattgctcaagaacaaggttctttgtcatcttctgaa |
| | gcagaaggtatggtcaaaaacttgcaaactactggtagatacttgagagatgtctggtaa |
| VALC | atggccgagatgttcaacggcaactcttctaacgacggatcttcttgcatgcccgtgaaggacgccctgcgac |
| (SEQ ID NO: 102) | gaaccggcaaccaccaccccaacctgtggaccgacgacttcatccagtctctgaactctccctactctgactc |
| | ttcttaccacaagcaccgagagatcctgatcgacgagatccgagacatgttctctaacggcgagggcgacga |
| | gttcggcgtgctcgagaacatctggttcgtggacgtggtgcagcgactgggcatcgaccgacacttccagga |
| | ggagatcaagaccgccctggactacatctacaagttctggaaccacgactctatcttcggcgacctgaacatg |
| | gtggccctgggcttccgaatcctgcgactgaaccgatacgtggcctcttctgacgtgttcaagaagttcaagg |
| | gcgaggagggccagttctctggcttcgagtcctctgaccaggacgctaagctcgaaatgatgctgaacctgt |
| | acaaggcctctgagctggacttccccgacgaggacatcctgaaggaggcccgagccttcgcctctatgtacc |
| | tgaagcacgtgatcaaggagtacggcgacatccaggagtctaagaaccccctgctgatggagatcgagtac |
| | accttcaagtacccctggcgatgccgactgccccgactcgaggcctggaacttcatccacatcatgcgacag |
| | caggactgcaacatctctctggccaacaacctctacaagatccccaagatctacatgaagaagatcctcgagc |
| | tggccatcctggacttcaacatcctgcagtctcagcaccagcacgagatgaagctgatctctacctggtggaa |
| | gaactcttctgctatccagctggacttcttccgacaccgacacatcgagtcttacttttggtgggcctcgcccct |
| | gttcgagcccgagttctctacctgccgaatcaactgcaccaagctgtctaccaagatgttcctgctggacgaca |
| | tctacgacacctacggcaccgtcgaggagctgaagcccttcaccaccaccctgacccgatgggacgtgtcta |
| | ccgtggacaaccaccccgactacatgaagatcgccttcaacttctcttacgagatctacaaggagatcgcctct |
| | gaggccgagcgaaagcacggccccttcgtgtacaagtacctgcagtcttgctggaagtcttacatcgaggcc |
| | tacatgcaggaggccgagtggatcgcctctaaccacatccccggcttcgacgagtacctgatgaacggcgt |
| | gaagtcctctggcatgcgaatcctgatgatccacgccctgatcctgatggacacccccctgtctgacgagatt |
| | ctcgagcagctggacatcccctcgtctaagtctcaggccctgctgtctctgatcacccgactggtggacgacg |
| | tgaaggacttcgaggacgagcaggcccacggcgagatggcctcactatcgagtgctacatgaaggacaac |
| | cacggctctacccgagaggacgccctgaactacctgaagatccgaatcgagtcttgcgtgcaggagctgaa |
| | caaggagctgctcgagccctctaacatgcacggatctttccgaaacctgtacctgaacgtgggaatgcgagt |
| | gattacttcatgctgaacgacggcgacctgttcacccactctaaccgaaaggagatccaggacgccatcacc |
| | aagttcttcgtcgagcccatcatcccctga |
| HmCYP | atgcaattcttctccttggtttccatcttcttgttcttgtccatttgtttttgttgagaaagtggaagaactccaactcc |
| (SEQ ID NO: 103) | caatctaaaaagagccaccaggtccatggaaattgccattattgggactatgagcatatggaggtggatgcc |
| | acatcatgattgagagataggctaaaaagtacggtccattgatgcacttgcaattgggtgaagtactgctgag |
| | ttgttacttctccagatatggccaaagaagttttgaaaacccacgatattgctttcgcttctagaccaaagttgttg |
| | gctccagaaatcgtagttacaacagatctgatattgccactgtccatacggtgattattggagacaaatgagaa |
| | agatctgcgtcttggaagattgtctgctaagaacgtcagatccttcagttccattagaagagatgaagtcttgag |
| | attggtcaacttcgttagatcactacttccgaaccagttaacttcaccgaaagattattcttgacacctcactatg |
| | acctgtagatctgcttaggtaaggattcaaagaacaagaaaccacatccaattgatcaaagaagtcattggat |
| | ggctggtggattgatgagctgatattacccatccttgaagacttgcatgtcttgactggtatggaaggtaagat |
| | tatgaaggcccatcataaggagatgccatcgttgaagatgttatcaacgaacacaaaaagaacttggctatgg |
| | gtaagactaatggtgctttgggtggtgaagatttgatcgatgttttgttaagattgatgaacgatggtggtttacaa |
| | ttcccaatcaccaacgataacattaaggccatcatcttcgatatgtttgctgctggtactgaaacttcttcctctact |
| | ttggtttgggctatggttcaaatgatgagaaacccaactattttggctaaggctcaagctgaagttagagaagct |
| | tttaagggtaaagaaactttcgacgaaaacgacgtcgaagaattgaagtacttgaagttggttatcaaagaaac |
| | attgagattgcacccaccagaccatgaggaccaagagaatgtagagaagaaaccgaaatcaacggttaca |
| | ccattccagttaagaccaaggttatggttaatgtttgggctttgggtagagatccaaagtattgggatgatgctg |
| | ataacttcaagccagaaagattcgaacaatgctccgttgactttatcggtaacaacttcgaatacttgccatttgg |
| | tggtggtagaagaatagtccaggtatttcatcggtaggccaatgatatttgccattggctcaattgagtaccac |
| | ttcgattggaaattaccaacaggtatggaacctaaggatttggatttgactgaattggtcggtattaccattgcca |
| | gaaagtccgatttgatgttagttgctactccataccaaccatccagagaataa |
| AtCPR | atgacttctgccttgtatgcctctgatttgttcaagcaattgaagtccattatgggtactgactcattgtccgatgat |
| (SEQ ID NO: 104) | gttgttttggttattgctactacctccttggctttggttgctggttttgttgttttattgtggaaaaagaccaccgccg |
| | atagatctggtgaattgaaaccattgatgatcccaaagtctttgatggccaaagatgaagatgatgatttggattt |
| | gggaccggtaagactagagtactattacttcggtactcaaaccggtactgctgaaggattgctaaagctagt |
| | ctgaagaaatcaaggccagatacgaaaaagctgccgttaaggttatagatttggatgattatgctgccgatgac |
| | gaccaatacgaagaaaagttgaagaaagaaaccttggccttcttctgtgttgctacttatggtgatggtgaacct |
| | actgataatgctgctagatatacaagtggacactgaagaaaacgaaagagacatcaagttgcaacaattggc |
| | ttacggtgatttgctagggtaacagacaatacgaacacttcaacaagatcggtatcgattggatgaagaattgt |
| | gtaagaagggtgccaagagattgattgaagttggtttgggtgatgatgaccaatccatcgaagatgattttaac |
| | gcctggaaagaatccttgtggtctgaattggataagttgagaaggacgaagatgacaaatctgagctacacc |
| | atacactgctgttatcccagaatatagagttgttacccatgatccaagattcaccactcaaaagtctatggaatct |
| | aacgttgctaacggtaacaccaccatcgatattcatcatccatgtagagttgatgtcgccgtccaaaaagaattg |
| | catactcatgaatctgacagatcctgcatccatttggaattcgatatttccagaaccggtattacttacgaaaccg |
| | gtgatcatgaggtgatacgctgaaaatcacgttgaaatcgttgaagaagccggtaagttgttaggtcattcctt |
| | agataggattctccatccatgccgacaaagaagatggactccattggaatctgctgaccaccaccatacca |
| | ggtccatgtactttgggtactggtttggctagatatgctgacttgttgaatccaccaagaaagtctgctttagttgc |
| | taggctgcttatgctactgaaccatctgaagccgaaaaattgaaacatttgacttccccagatggtaaggacga |
| | atattctcaatggatagttgcctcccaaagatccttgaggaagttatggctgcattccatctgctaaaccaccatt |
| | gggtgattattgctgctattgctccaagattgcaacctagatattactccatacctccagtccaagattagctcc |
| | atcaagagttcatgttacatccgctaggatatggtccaactccaactggtagaattcataagggtgatgactac |
| | ctggatgaagaacgctgttccagctgaaaaatctcatgaatgttctggtgccccaattttcattagagcttctaatt |
| | tcaagagccatccaacccatctactccaatagttatggaggtccaggtacaggatagctccattagaggatc |
| | ttacaagaaagaatggccttgaaagaagatggtgaagaattgggttcctccttgttgttttttggttgcagaaaca |
| | gacaaatggatttcatctatgaagatgaattgaacaacttcgtcgaccaaggtgttatctccgaattgattatggc |
| | cattctagagaaggtgctcaaaaagaatacgtccaacacaagatgatggaaaaagccgctcaagtagggac |
| | ttgatcaaagaagaaggttacttgtacgtttgcggtgatgctaaaggtatggctagagatgttcatagaacattg |
| | cataccatcgttcaagaacaagaaggtgtctcatcactgaagctgaagctatcgttaagaagagcaaactga |
| | aggtagatacttgagagatgtctggtaa |

### Example 9

### Production of Alkaloids and Flavonoids by the Co-Culture System

Another major class of natural products that can be produced using cellular consortia are alkaloids, which are derived from aromatic amino acids that can cross cellular membranes (Nakagawa, et al. Nat. Commun. (2011)2:326). E. coli is engineered to overproduce an aromatic amino acid, e.g. tyrosine, and S. cerevisiae is manipulated to functionalize the amino acid into a product, e.g. (S)-reticuline, an important precursor of benzylisoquinoline alkaloids (including >2,500 molecules) (Nakagawa, et al. Nat. Commun. (2011) 2:326; Glen, et al. Curr. Opin. Biotechnol. (2013)24:354-365). By doing so, the whole process is modular, i.e. constructing the downstream alkaloid pathway in S. cerevisiae does not negatively affect the upstream amino acid production in E. coli. In addition, it is advantageous to produce amino acids in bacteria due to their fast growth rate, and to reconstruct the downstream pathway for alkaloids in yeasts because the involved enzymes are usually from plants and better expressed in yeasts in terms of activity (Santos, et al. PNAS (2012)109:13538-13543; Nakagawa, et al. Nat. Commun. (2011) 2:326). Additionally, using this co-culture system xylose is used as sole carbon source is used by E. coli, which produces acetate for the S. cerevisiae strain to grow.

Like alkaloids, flavonoids (including >8,000 molecules) are also derived from aromatic amino acids (Trantas, et al. Met. Engin. (2009)11:355-366). The difference is that synthesis of flavonoids also requires malonyl-CoA, which can be readily produced from acetate via acetyl-CoA. Therefore, the above co-culture design for production of alkaloids can also be applied to that of flavonoids. Plus, as an additional advantage the S. cerevisiae strain would have ample substrates for producing malonyl-CoA as it grows on acetate.

### Example 10

### Production of Short Chain Dicarboxylic Acids by the Co-Culture System

Another type of compounds can be produced using a cellular consortium is short chain dicarboxylic acids (C6-C10), which can be produced from short chain fatty acids via ω-oxidation (Craft, et al. Appl. and Environ. Microbiol. (2003)69:5983-5991). It has been demonstrated that E. coli is superior to yeasts in terms of short chain fatty acid production, because yeast fatty acid synthases are far more complex than the bacterial counterparts, making early termination of the fatty acid chain elongation to be much more difficult in yeasts (Choi, et al. Nature (2013)502:571-574; Leber, et al. Biotechnol. and Bioeng. (2014)111:347-358). On the other hand, yeasts are very efficient in carrying out fatty acid oxidation as they are better hosts than bacteria for expressing cytochrome P450s and contain peroxisome, which is an organelle specialized in fatty acid oxidation (Craft, et al. Appl. and Environ. Microbiol. (2003)69:5983-5991). A very stable co-culture that is efficient in producing short chain dicarboxylic acids is established by engineering E. coli to produce short chain fatty acids from xylose, and engineering S. cerevisiae to oxidize the fatty acids. This co-culture results in production of short chain dicarboxylic acids which can be polymerized into many key industrial polymers, e.g. Nylon.

### Example 11

### Production of Recombinant Proteins by the Co-Culture System

The E. coli-S. cerevisiae co-culture systems described herein can also be designed to produce recombinant proteins. Recombinant proteins from microbes have a significant share in current biotech industry. The global market of E. coli-produced Insulin was valued at USD 20 billion in 2012 (www.marketwatch.com). A major constraint of recombinant protein production in E. coli has been accumulation of acetate, which is known to inhibit cell growth (Eiteman, et al. Trends in Biotech. (2006)24:530-536). This problem is solved by co-culturing a S. cerevisiae with a recombinant-protein-producing E. coli in the medium which contains xylose as sole carbon source, because the S. cerevisiae consumes all the acetate produced by the E. coli. The S. cerevisiae in this case can also be engineered to produce the same recombinant protein as the E. coli strain, further converting the undesired acetate into a useful, desired product.

### REFERENCES

Agapakis, C. M., Boyle, P. M. & Silver, P. A. Natural strategies for the spatial optimization of metabolism in synthetic biology. Nat Chem Biol 8, 527-535 (2012).
Ajikumar, P. K. et al. Isoprenoid pathway optimization for Taxol precursor overproduction in Escherichia coli. Science 330, 70-74 (2010).
Ajikumar, P. K. et al. Terpenoids: opportunities for biosynthesis of natural product drugs using engineered microorganisms. Mol Pharm 5, 167-190 (2008).
Alonso-Gutierrez, J. et al. Metabolic Engineering of Escherichia coli for Limonene and Perillyl Alcohol Production. Metabolic engineering (2013).
Artsatbanov, V. Y. et al. Influence of Oxidative and Nitrosative Stress on Accumulation of
Diphosphate Intermediates of the Non-mevalonate Pathway of Isoprenoid Biosynthesis in Corynebacteria and Mycobacteria. Biochemistry. Biokhimiia 77, 362-371 (2012).
Avalos, J. L., Fink, G. R. & Stephanopoulos, G. Compartmentalization of metabolic pathways in yeast mitochondria improves the production of branched-chain alcohols. Nature biotechnology 31, 335-341 (2013).
Bayer, T. S. et al. Synthesis of methyl halides from biomass using engineered microbes. Journal of the American Chemical Society 131, 6508-6515 (2009).
Blazeck, J., Garg, R., Reed, B. & Alper, H. S. Controlling promoter strength and regulation in
Saccharomyces cerevisiae using synthetic hybrid promoters. Biotechnology and bioengineering 109, 2884-2895 (2012).
Causey, T. B., Zhou, S., Shanmugam, K. T. & Ingram, L. O. Engineering the metabolism of Escherichia coli W3110 for the conversion of sugar to redox-neutral and oxidized products: homoacetate production. Proceedings of the National Academy of Sciences of the United States of America 100, 825-832 (2003).
Choi, Y. J. & Lee, S. Y. Microbial production of short-chain alkanes. Nature 502, 571-574 (2013).
Craft, D. L., Madduri, K. M., Eshoo, M. & Wilson, C. R. Identification and characterization of the CYP52 family of Candida tropicalis ATCC 20336, important for the conversion of fatty acids and alkanes to alpha,omega-dicarboxylic acids. Applied and environmental microbiology 69, 5983-5991 (2003).
Davison, B. H. & Stephanopoulos, G. Effect of pH oscillations on a competing mixed culture. Biotechnology and bioengineering 28, 1127-1137 (1986).
De Virgilio, C. et al. Cloning and disruption of a gene required for growth on acetate but not on ethanol: the acetyl-coenzyme A synthetase gene of Saccharomyces cerevisiae. Yeast 8, 1043-1051 (1992).
Doshi, R., Nguyen, T. & Chang, G. Transporter-mediated biofuel secretion. Proceedings of the National Academy of Sciences of the United States of America 110, 7642-7647 (2013).
Eiteman, M. A. & Altman, E. Overcoming acetate in Escherichia coli recombinant protein fermentations. Trends in biotechnology 24, 530-536 (2006).
Eiteman, M. A., Lee, S. A. & Altman, E. A co-fermentation strategy to consume sugar mixtures effectively. J Biol Eng 2, 3 (2008).
Eiteman, M. A., Lee, S. A., Altman, R. & Altman, E. A substrate-selective co-fermentation strategy with Escherichia coli produces lactate by simultaneously consuming xylose and glucose. Biotechnology and bioengineering 102, 822-827 (2009).
Flagfeldt, D. B., Siewers, V., Huang, L. & Nielsen, J. Characterization of chromosomal integration sites for heterologous gene expression in Saccharomyces cerevisiae. Yeast 26, 545-551 (2009).
Fredrickson, A. G. & Stephanopoulos, G. Microbial competition. Science 213, 972-979 (1981).
Glenn, W. S., Runguphan, W. & O'Connor, S. E. Recent progress in the metabolic engineering of alkaloids in plant systems. Current opinion in biotechnology 24, 354-365 (2013).
Goyal, G., Tsai, S. L., Madan, B., DaSilva, N. A. & Chen, W. Simultaneous cell growth and ethanol production from cellulose by an engineered yeast consortium displaying a functional mini-cellulosome. Microbial cell factories 10, 89 (2011).
Guerra-Bubb, J., Croteau, R. & Williams, R. M. The early stages of taxol biosynthesis: an interim report on the synthesis and identification of early pathway metabolites. Natural product reports 29, 683-696 (2012).
Guo, J. et al. CYP76AH1 catalyzes turnover of miltiradiene in tanshinones biosynthesis and enables heterologous production of ferruginol in yeasts. Proceedings of the National Academy of Sciences of the United States of America 110, 12108-12113 (2013).
Hefferon, K. Plant-derived pharmaceuticals for the developing world. Biotechnol J 8, 1193-1202 (2013).
Hefner, J. et al. Cytochrome P450-catalyzed hydroxylation of taxa-4(5),11(12)-diene to taxa-4(20),11(12)-dien-5alpha-ol: the first oxygenation step in taxol biosynthesis. Chemistry & biology 3, 479-489 (1996).
Jennewein, S., Long, R. M., Williams, R. M. & Croteau, R. Cytochrome p450 taxadiene 5alpha-hydroxylase, a mechanistically unusual monooxygenase catalyzing the first oxygenation step of taxol biosynthesis. Chemistry & biology 11, 379-387 (2004).
Kratzer, S. & Schuller, H. J. Transcriptional control of the yeast acetyl-CoA synthetase gene, ACS1, by the positive regulators CAT8 and ADR1 and the pleiotropic repressor UME6. Molecular microbiology 26, 631-641 (1997).
Leber, C. & Da Silva, N. A. Engineering of Saccharomyces cerevisiae for the synthesis of short chain fatty acids. Biotechnology and bioengineering 111, 347-358 (2014).
Melnik, S. & Stoger, E. Green factories for biopharmaceuticals. Current medicinal chemistry 20, 1038-1046 (2013).
Minty, J. J. et al. Design and characterization of synthetic fungal-bacterial consortia for direct production of isobutanol from cellulosic biomass. Proceedings of the National Academy of Sciences of the United States of America 110, 14592-14597 (2013).
Nakagawa, A. et al. A bacterial platform for fermentative production of plant alkaloids. Nat Commun 2, 326 (2011).
Nowak, M. A. Five rules for the evolution of cooperation. Science 314, 1560-1563 (2006)
Paddon, C. J. et al. High-level semi-synthetic production of the potent antimalarial artemisinin. Nature 496, 528-532 (2013).
Pillai, V. C., Snyder, R. O., Gumaste, U., Thekkumkara, T. J. & Mehvar, R. Effects of transient overexpression or knockdown of cytochrome P450 reductase on reactive oxygen species generation and hypoxia reoxygenation injury in liver cells. Clin Exp Pharmacol Physiol 38, 846-853 (2011).
Reed, J. R., Cawley, G. F. & Backes, W L Inhibition of cytochrome P450 1A2-mediated metabolism and production of reactive oxygen species by heme oxygenase-1 in rat liver microsomes. Drug Metab Lett 5, 6-16 (2011).
Rontein, D. et al. CYP725A4 from yew catalyzes complex structural rearrangement of taxa-4(5),11(12)-diene into the cyclic ether 5(12)-oxa-3(11)-cyclotaxane. The Journal of biological chemistry 283, 6067-6075 (2008).
Santos, C. N., Xiao, W. & Stephanopoulos, G. Rational, combinatorial, and genomic approaches for engineering L-tyrosine production in Escherichia coli. Proceedings of the National Academy of Sciences of the United States of America 109, 13538-13543 (2012).
Schoendorf, A., Rithner, C. D., Williams, R. M. & Croteau, R. B. Molecular cloning of a cytochrome P450 taxane 10 beta-hydroxylase cDNA from Taxus and functional expression in yeast. Proceedings of the National Academy of Sciences of the United States of America 98, 1501-1506 (2001).
Smid, E. J. & Lacroix, C. Microbe-microbe interactions in mixed culture food fermentations. Current opinion in biotechnology 24, 148-154 (2013).
Sun, J. et al. Cloning and characterization of a panel of constitutive promoters for applications in pathway engineering in Saccharomyces cerevisiae. Biotechnology and bioengineering 109, 2082-2092 (2012).
Trantas, E., Panopoulos, N. & Ververidis, F. Metabolic engineering of the complete pathway leading to heterologous biosynthesis of various flavonoids and stilbenoids in Saccharomyces cerevisiae. Metabolic engineering 11, 355-366 (2009).
Walker, K., Schoendorf, A. & Croteau, R. Molecular cloning of a taxa-4(20),11(12)-dien-5alpha-ol-O-acetyl transferase cDNA from Taxus and functional expression in Escherichia coli. Archives of biochemistry and biophysics 374, 371-380 (2000).
Wheeler, A. L. et al. Taxol biosynthesis: differential transformations of taxadien-5 alpha-ol and its acetate ester by cytochrome P450 hydroxylases from Taxus suspension cells. Archives of biochemistry and biophysics 390, 265-278 (2001).
Wriessnegger, T. et al. Production of the sesquiterpenoid (+)-nootkatone by metabolic engineering of Pichia pastoris. Metabolic engineering 24C, 18-29 (2014).
Xia, T., Eiteman, M. A. & Altman, E. Simultaneous utilization of glucose, xylose and arabinose in the presence of acetate by a consortium of Escherichia coli strains. Microbial cell factories 11, 77 (2012).
Xue, J. & Ahring, B. K. Enhancing Isoprene Production by Genetic Modification of the 1-Deoxy-D-Xylulose-5-Phosphate Pathway in Bacillus subtilis. Applied and environmental microbiology 77, 2399-2405 (2011).
Zhou, Y. J. et al. Modular pathway engineering of diterpenoid synthases and the mevalonic acid pathway for miltiradiene production. Journal of the American Chemical Society 134, 3234-3241 (2012).
Zhou, K., Zou, R., Zhang, C., Stephanopoulos, G. & Too, H. P. Optimization of amorphadiene synthesis in Bacillus subtilis via transcriptional, translational, and media modulation. Biotechnology and bioengineering (2013).
Zou, R., Zhou, K., Stephanopoulos, G. & Too, H. P. Combinatorial Engineering of 1-Deoxy-D-Xylulose 5-Phosphate Pathway Using Cross-Lapping In Vitro Assembly (CLIVA) Method. PloS one 8, e79557 (2013).

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only. Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety for the specific purpose mentioned herein.

What is claimed is:

1. A synthetic cellular consortium, comprising
a first organism comprising a first part of a biosynthetic pathway that produces a first compound and
a second organism comprising a second part of the biosynthetic pathway that is able to convert the first compound into a second compound; and
optionally comprising a third organism that converts the second compound into a third compound.

2. The synthetic cellular consortium of claim 1, wherein the first and/or second organism is
a bacterium, optionally wherein the bacterium is Escherichia coli, Bacillus subtilis, or Bacillus megaterium, optionally wherein the E. coli, B. subtilis or B. megaterium is genetically engineered;
a yeast, optionally wherein the yeast is Saccharomyces cerevisiae, Yarrowia lipolytica, or Pichia pastoris, optionally wherein the S. cerevisiae, Y. lipolytica, or P. pastoris is genetically engineered; or
a plant cell, optionally wherein the plant cell belongs to the genus Taxus, optionally wherein the Taxus cell is induced with methyl jasmonate, optionally wherein the Taxus cell is genetically engineered.

3-4. (canceled)

5. The synthetic cellular consortium of claim 1, wherein the first organism recombinantly expresses one or more enzymes of a biosynthetic pathway, optionally the shikimate pathway or a secondary metabolite biosynthetic pathway, optionally wherein the secondary metabolite biosynthetic pathway is an isoprenoid biosynthetic pathway, optionally a 2-C-methyl-D-erythritol 4-phosphate/1-deoxy-D-xylulose 5-phosphate (MEP) pathway.

6-8. (canceled)

9. The synthetic cellular consortium of claim 5, wherein the first organism recombinantly expresses
(i) any of the genes dxs, idi, ispD, ispF of the MEP pathway, and/or any of the genes ispG and ispH of the MEP pathway, optionally wherein the genes of the MEP pathway are isolated from E. coli;
(ii) a geranylgeranyl diphosphate synthase (GGPPS), optionally wherein a nucleic acid encoding GGPPS is isolated from T. canadensis;
(iii) a taxadiene synthase (TS), optionally wherein a nucleic acid encoding TS is isolated from T. brevifolia;
optionally wherein one or more of the nucleic acids encoding enzymes of the MEP pathway, GGPPS or TS are integrated into the genome at a specific site or on a plasmid;
optionally wherein expression of one or more of the nucleic acids is under control of a constitutively active promoter, optionally the bacteriophage T7 promoter; optionally wherein one of more of the nucleic acid encoding genes are codon optimized for expression in E. coli.

10-20. (canceled)

21. The synthetic cellular consortium of claim 5, wherein the biosynthetic pathway is the shikimate pathway and the genes ydiB and/or aroE are mutated or deleted from the first organism, and/or wherein the first organism expresses one or more global transcription machinery genes, optionally rpoA, optionally wherein the sequence of rpoA comprises one or more mutations; and/or
(ii) the genes encoding F₁F₀ H⁺-ATP synthase subunits are mutated or deleted from the first organism, optionally wherein the genes encoding F₁F₀ H⁺-ATP synthase subunits that are mutated or deleted are atpFH.

22-25. (canceled)

26. The synthetic cellular consortium of claim 1, wherein genes encoding F₁F₀ H⁺-ATP synthase subunits are mutated or deleted from the first organism, optionally wherein the genes encoding F₁F₀ H⁺-ATP synthase subunits that are mutated or deleted are atpFH.

27-33. (canceled)

34. The synthetic cellular consortium of claim 1, wherein the first organism recombinantly expresses the genes KSL and CPS, optionally Salvia miltiorrhiza genes; or a sesquiterpene synthase, optionally encoded by a Callitropsis nootkatensis gene.

35-37. (canceled)

38. The synthetic cellular consortium of claim 1, wherein the second organism recombinantly expresses one or more enzymes of a biosynthetic pathway, wherein the biosynthetic pathway is optionally
(i) a secondary metabolite biosynthetic pathway, optionally wherein the secondary metabolite biosynthetic pathway is an isoprenoid biosynthetic pathway, a polyketide biosynthetic pathway or an alkaloid biosynthetic pathway;
(ii) a pathway for the production of a monoacetylated deoxygenated taxane;
(iii) a pathway for the production of ferruginol;
(iv) a pathway for the production of nootkatone;
(v) a pathway for the production of an aromatic compound or aromatic-derived compound, optionally wherein the aromatic compound is 3-aminobenzoate or p-hydroxybenzoate (PHB), optionally wherein the aromatic-derived compound is muconic acid, an alkaloid, or a flavonoid; and/or
(vi) a pathway for the production of short chain dicarboxylic acids.

39. The synthetic cellular consortium of claim 38, wherein
(i) the second organism recombinantly expresses components of an oxidoreductase, components of an acyltransferase or an enzyme catalyzing hydroxylation;
(ii) the biosynthetic pathway is for production of a monoacetylated deoxygenated taxane and the second organism recombinantly expresses a taxadien-5αol acetyl transferase and a taxane 10β hydroxylase, optionally wherein the taxadien-5αol acetyl transferase and/or the taxane 10β hydroxylase is isolated from Taxus cuspidate;
(iii) the biosynthetic pathway is for production of ferruginol and the second organism recombinantly expresses the genes CYP and CPR, optionally wherein the genes CYP and CPR are Salvia miltiorrhiza genes,
(iv) the biosynthetic pathway is for production of nootkatone and the second organism recombinantly expresses the genes CYP and CPR, optionally wherein the CYP gene is a Hyoscyamus muticus gene and/or the CPR gene is a Arabidopsis thaliana gene;
(v) the biosynthetic pathway is a muconic acid biosynthetic pathway and the second organism recombinantly expresses one or more of the genes aroZ, aroY and catA;
(vi) the biosynthetic pathway is a PHB biosynthetic pathway and the second organism recombinantly expresses one or more of the genes one or more of the genes aroE, ydiB, aroL, aroA, aroC and ubiC;
(vi) the biosynthetic pathway is a 3-aminobenzoate biosynthetic pathway and the second organism recombinantly expresses pctV; and/or
(vii) the second organism recombinantly expresses shiA.

40-41. (canceled)

42. The synthetic cellular consortium of claim 38, wherein the biosynthetic pathway is the isoprenoid pathway and the second organism recombinantly expresses components of a cytochrome P450, optionally a taxadiene 5α hydroxylase and NADPH-cytochrome P450 reductase, optionally wherein the taxadiene 5α hydroxylase and NADPH-cytochrome P450 reductase are expressed as a single polypeptide, and/or optionally wherein the taxadiene 5α hydroxylase and/or NADPH-cytochrome P450 reductase is isolated from T. cuspidate; and/or optionally wherein a nucleic acid encoding taxadiene 5α hydroxylase and NADPH-cytochrome P450 reductase is integrated into the genome at a specific site; and/or optionally wherein a nucleic acid encoding taxadiene 5α hydroxylase and NADPH-cytochrome P450 reductase is on a plasmid; and/or optionally wherein expression of the nucleic acid encoding taxadiene 5α hydroxylase and NADPH-cytochrome P450 reductase is driven by a TEF promoter, an UAS-GPD promoter, a GPD promoter, or an ACS promoter.

43-68. (canceled)

69. The synthetic cellular consortium of claim 1, wherein a carbon source utilized by the first organism comprises xylose, glucose and/or glycerol; and/or
wherein the second organism can utilize a carbon metabolic byproduct produced by the first organism, optionally wherein the carbon metabolic byproduct produced by the first organism is acetate; and/or
wherein a carbon source utilized by the second organism comprises xylose, glucose, and/or glycerol, optionally wherein the carbon source utilized by the first organism is a different carbon source than the carbon source utilized by the second organism; and/or
wherein the first compound produced by the first organism comprises at least part of the second compound produced by the second organism; and/or wherein the first compound produced by the first organism is membrane permeable or transported out of the first organism.

70-75. (canceled)

76. The synthetic cellular consortium of claim 1, wherein the first compound produced by the first organism is an intermediate of the isoprenoid pathway, optionally wherein the isoprenoid intermediate is
(i) taxadiene or an oxygenated taxane, optionally wherein the oxygenated taxane is taxadien-5a-ol, taxadien-5a-ol-10b-ol or taxadiene-5a-acetate-10b-ol, or wherein the second organism converts the isoprenoid intermediate produced by the first organism into an oxygenated taxane or acetylated taxane;
(ii) miltiradiene; optionally wherein the second organism converts the miltiradiene produced by the first organism into ferruginol; or
(iii) valencene, optionally wherein the second organism converts the valencene produced by the first organism into nootkatone.

77-84. (canceled)

85. The synthetic cellular consortium of claim 1, wherein the first compound produced by the first organism is
(i) an intermediate of the shikimate pathway, optionally dehydroshikimate (DHS) and optionally wherein the second organism converts DHS produced by the first organism into an aromatic compound or an aromatic-derived compound, wherein the aromatic compound is optionally p-hydroxybenzoate or 3-aminobenzoate, or wherein the aromatic-derived compound is optionally muconic acid;
(ii) an aromatic amino acid, optionally wherein the second organism converts the aromatic amino acid produced by the first organism into an alkaloid or a flavonoid; or
(iii) a recombinant protein; and/or
wherein the second organism can utilize a carbon metabolic byproduct produced by the first organism.

86-95. (canceled)

96. The synthetic cellular consortium of claim 1, wherein the second organism produces a recombinant protein, optionally wherein the recombinant protein produced by the second organism is the same as the recombinant protein produced by the first organism.

97. (canceled)

98. A method of synthesizing a compound, comprising culturing the synthetic microbial consortium of claim 1, optionally wherein the synthetic cellular consortium is cultured in a bioreactor or a shake flask, and/or optionally further comprising isolating or purifying the second compound.

99-100. (canceled)

101. The method of claim 98, wherein the method further comprises isolating or purifying the second compound, wherein the second compound is an oxygenated taxane, optionally wherein the culture comprises 20-25000 mg/L oxygenated taxanes;
acetylated taxane;
ferruginol, optionally wherein the supernatant of the culture comprises 10-25000 mg/L ferruginol;
nootkatone, optionally wherein the supernatant of the culture comprises 10-25000 mg/L nootkatone;
an aromatic compound, optionally wherein the aromatic compound is PHB, optionally wherein a supernatant of the culture comprises at least 50 mg/L PHB, or 3-aminobenzoate, optionally wherein a culture supernatant of the culture comprises at least 3 mg/mL 3-aminobenzoate;
an aromatic-derived compound, optionally wherein the aromatic-derived compound is muconic acid, optionally wherein a supernatant of the culture comprises at least 400 mg/L muconic acid, an alkaloid, optionally wherein a supernatant of the culture comprises at least 100 mg/L alkaloid, or a flavonoid, optionally wherein a supernatant of the culture comprises at least 100 mg/L flavonoid; or
a short chain dicarboxylic acid, optionally wherein a supernatant of the culture comprises at least 100 mg/L short chain dicarboxylic acids.

102-118. (canceled)

119. A culture comprising the synthetic cellular consortium of claim 1.

120. A method of synthesizing a compound, comprising culturing cells of a first organism comprising a first part of a biosynthetic pathway that produces a first compound,
isolating the first compound from the culture of the first organism,
separately culturing cells of a second organism comprising a second part of the biosynthetic pathway that converts the first compound into a second compound, and
adding the isolated first compound to the culture of the second organism; and
optionally isolating or purifying the second compound, optionally isolating the second compound from the culture of the second organism.

121. (canceled)

122. The method of claim 120, wherein the first and/or second organism is
a bacterium, optionally Escherichia coli, Bacillus subtilis or Bacillus megaterium, optionally wherein the Escherichia coli, Bacillus subtilis or Bacillus megaterium is genetically engineered, optionally wherein the E. coli is an E. coli K12 derivative or an E. coli B derivative;
a yeast, optionally wherein the yeast is Saccharomyces cerevisiae, Yarrowia lipolytica, or Pichia pastoris, optionally wherein the S. cerevisiae, Y. lipolytica, or P. pastoris is genetically engineered; or
a plant cell, optionally wherein the plant cell belongs to the genus Taxus, optionally wherein the Taxus cell is induced with methyl jasmonate, optionally wherein the Taxus cell is genetically engineered.

123-125. (canceled)

126. The method of claim 120, wherein the first organism recombinantly expresses one or more enzymes of a biosynthetic pathway, optionally the shikimate pathway or a secondary biosynthetic pathway, optionally wherein the secondary biosynthetic pathway is an isoprenoid biosynthetic pathway, optionally the MEP pathway.

127-129. (canceled)

130. The method of claim 120, wherein the biosynthetic pathway is the MEP pathway and wherein the first organism recombinantly expresses
(i) the genes dxs, idi, ispD, ispF of the MEP pathway, and/or any of the genes ispG and ispH of the MEP pathway, optionally wherein the genes of the MEP pathway are isolated from E. coli;
(ii) a geranylgeranyl diphosphate synthase (GGPPS), optionally wherein a nucleic acid encoding GGPPS is isolated from T. canadensis; or
(iii) a sesquiterpene synthase, optionally wherein the sesquiterpene synthase is encoded by a Callitropsis nootkatensis gene;
optionally wherein one or more of the nucleic acids encoding enzymes of the MEP pathway, GGPPS or TS is integrated into the genome at a specific site or on a plasmid; optionally wherein expression of one or more of the nucleic acids is under control of a constitutively active promoter, optionally the bacteriophage T7 promoter.

131-140. (canceled)

141. The method of claim 126, wherein the first organism recombinantly expresses the genes KSL and CPS, optionally Salvia miltiorrhiza genes; or a sesquiterpene synthase, optionally encoded by a Callitropsis nootkatensis gene.

142-145. (canceled)

146. The method of claim 120, wherein the genes ydiB and/or aroE are mutated or deleted from the first organism, and/or wherein the first organism expresses one or more global transcription machinery genes, and/or wherein any or all of the nucleic acids encoding genes are codon optimized for expression in E. coli.

147-155. (canceled)

156. The method of claim 120, wherein the second organism recombinantly expresses one or more enzymes of a biosynthetic pathway, wherein the biosynthetic pathway is optionally
(i) a secondary biosynthetic pathway, optionally wherein the secondary metabolite biosynthetic pathway is an isoprenoid biosynthetic pathway, a polyketide biosynthetic pathway, or an alkaloid biosynthetic pathway;
(ii) a biosynthetic pathway for the production of an aromatic compound or an aromatic-derived compound, optionally wherein the aromatic compound is 3-aminobenzoate or p-hydroxybenzoate (PHB), or optionally wherein the aromatic-derived compound is muconic acid, an alkaloid, or a flavonoid;
(iii) a pathway for the production of an aromatic compound or an aromatic-derived compound, optionally wherein the aromatic compound is 3-aminobenzoate or p-hydroxybenzoate (PHB), optionally wherein the aromatic-derived compound is muconic acid, an alkaloid, or a flavonoid.

157-158. (canceled)

159. The synthetic cellular consortium of claim 156, wherein the second organism recombinantly expresses
(i) components of an oxidoreductase, an acyltransferase or an enzyme catalyzing hydroxylation;
(ii) one or more of the genes aroZ, aroY and catA of a muconic acid biosynthetic pathway,
(iii) one or more of the genes aroE, ydiB, aroL, aroA, aroC and ubiC of the PHB biosynthetic pathway, or
(iv) pctV for the biosynthesis of 3-aminobenzoate; and/or
(v) shiA.

160. The method of claim 156, wherein the second organism recombinantly expresses components of a cytochrome P450, optionally a taxadiene 5α hydroxylase and NADPH-cytochrome P450 reductase, optionally wherein the second organism recombinantly expresses taxadiene 5α hydroxylase and NADPH-cytochrome P450 reductase as a single polypeptide, optionally wherein the second organism recombinantly expresses taxadiene 5α hydroxylase and NADPH-cytochrome P450 reductase with N-terminal membrane-binding domains, optionally wherein a nucleic acid encoding taxadiene 5α hydroxylase and NADPH-cytochrome P450 reductase is isolated from T. cuspidate; and/or optionally wherein a nucleic acid encoding taxadiene 5α hydroxylase and NADPH-cytochrome P450 reductase is integrated into the genome at a specific site, and/or optionally wherein expression of the nucleic acid encoding taxadiene 5α hydroxylase and NADPH-cytochrome P450 reductase is driven by a TEF promoter, an UAS-GPD promoter, a GPD promoter, or an ACS promoter.

161-177. (canceled)

178. The method of claim 120, wherein
the first compound produced by the first organism comprises at least part of the second compound produced by the second organism, and/or
the first compound produced by the first organism is membrane permeable or transported out of the first organism, and/or
the intermediate/first compound produced by the first organism is an intermediate of the isoprenoid pathway, optionally taxadiene or an oxygenated taxane, optionally wherein the oxygenated taxane is taxadien-5a-ol, taxadien-5a-ol-10b-ol or taxadien-5a-acetate-10b-ol; or wherein the first compound produced by the first organism is an intermediate of the shikimate pathway, optionally dehydroshikimate (DHS).

179-182. (canceled)

183. The method of claim 178, wherein
the second organism converts the isoprenoid intermediate produced by the first organism into an oxygenated taxane or acetylated taxane; or
the second organism converts DHS produced by the first organism into an aromatic compound or an aromatic-derived compound; or
the second organism converts DHS produced by the first organism into muconic acid, p-hydroxybenzoate or 3-amino benzoate.

184-190. (canceled)

191. A recombinant cell that expresses
(i) a DHS dehydratase (aroZ), a protocatechuic acid (PCA) decarboxylase (aroY), and a catechol 1,2-dioxygenase (catA), and in which the genes ydiB and aroE have been mutated or deleted;
(ii) a shikimate dehydrogenase (aroE), a shikimate kinase (aroL), a 5-enolpyruvyl shikimate 3-phosphate synthase (aroA), a chorismate synthase (aroC), and a chorismate pyruvate lyase (ubiC), and in which the genes ydiB and aroE have been mutated or deleted; or
(iii) an amino transferase (pctV) and in which the genes ydiB and aroE have been mutated or deleted; and
optionally wherein the cell further expresses a shikimate/DHS transporter (shiA) and/or one or more global transcription machinery genes, optionally wherein the global transcription machinery gene is rpoA, optionally wherein the sequence of rpoA comprises one or more mutations; optionally wherein the cell is a microbial cell, optionally an Escherichia coli cell, optionally an Escherichia coli BL21(DE3) cell.

192-200. (canceled)

201. A method of producing muconic acid, PHB, or 3-aminobenzoate, the method comprising culturing the cell of claim 191 to produce muconic acid, PHB, or 3-aminobenzoate, optionally wherein the method further comprises isolating and/or purifying the muconic acid, PHB, or 3-aminobenzoate, optionally wherein the cell culture contains at least 400 mg/L muconic acid or at least 3 mg/L 3-aminobenzoate.

202-212. (canceled)


SEQUENCE LISTING (105 sequences)

SEQ ID NO: 1 - DNA, 32 bp, Artificial Sequence
  Synthetic Polynucleotide
gctctagaaaaatggctctgttattagcagtt

SEQ ID NO: 2 - DNA, 49 bp, Artificial Sequence
  Synthetic Polynucleotide
gcaagcttttagtgatggtgatgatgatgccaaatatcccgtaagtagc

SEQ ID NO: 3 - DNA, 15 bp, Artificial Sequence
  Synthetic Polynucleotide
gccaggcgcctttat

SEQ ID NO: 4 - DNA, 58 bp, Artificial Sequence
  Synthetic Polynucleotide
gcaaaaggccaggaaccgtaaaaaggccgcgttgctggcgttttgcgaaaccctatgc

SEQ ID NO: 5 - DNA, 57 bp, Artificial Sequence
  Synthetic Polynucleotide
ggacggatcgcttgcctgtaacttacacgcgcctcgtatcaatggaaggtcgggatg

SEQ ID NO: 6 - DNA, 17 bp, Artificial Sequence
  Synthetic Polynucleotide
ataaagcagccgctacc

SEQ ID NO: 7 - DNA, 15 bp, Artificial Sequence
  Synthetic Polynucleotide
acgccagcaacgcgg

SEQ ID NO: 8 - DNA, 16 bp, Artificial Sequence
  Synthetic Polynucleotide
gatacgaggcgcgtgt

SEQ ID NO: 9 - DNA, 19 bp, Artificial Sequence
  Synthetic Polynucleotide
gtcgaccatcatcatcatc

SEQ ID NO: 10 - DNA, 19 bp, Artificial Sequence
  Synthetic Polynucleotide
atgttattcctccttattt

SEQ ID NO: 11 - DNA, 25 bp, Artificial Sequence
  Synthetic Polynucleotide
ggaataacatgtgtcccgtattatt

SEQ ID NO: 12 - DNA, 25 bp, Artificial Sequence
  Synthetic Polynucleotide
gatggtcgacttactgctgctgtgc

SEQ ID NO: 13 - DNA, 25 bp, Artificial Sequence
  Synthetic Polynucleotide
ggaataacatatgtcgagtaagtta

SEQ ID NO: 14 - DNA, 25 bp, Artificial Sequence
  Synthetic Polynucleotide
gatggtcgactcaggcagtcaggcg

SEQ ID NO: 15 - DNA, 25 bp, Artificial Sequence
  Synthetic Polynucleotide
actgggccttggcgtttaagggcac

SEQ ID NO: 16 - DNA, 25 bp, Artificial Sequence
  Synthetic Polynucleotide
ggagtcgcattggtgctacgcctgt

SEQ ID NO: 17 - DNA, 19 bp, Artificial Sequence
  Synthetic Polynucleotide
atgcgactcctgcattagg

SEQ ID NO: 18 - DNA, 19 bp, Artificial Sequence
  Synthetic Polynucleotide
aaggcccagtctttcgact

SEQ ID NO: 19 - DNA, 60 bp, Artificial Sequence
  Synthetic Polynucleotide
tagtaagcgttgcttttatttaaagagcaatatcagaacgttaacggctggagctgcttc

SEQ ID NO: 20 - DNA, 60 bp, Artificial Sequence
  Synthetic Polynucleotide
agcgcgccagagagaagctctgccatttgttcgtttttggttaccaattagccatggtcc

SEQ ID NO: 21 - DNA, 31 bp, Artificial Sequence
  Synthetic Polynucleotide
taccatgggcatgatgagcctggcatttaat

SEQ ID NO: 22 - DNA, 31 bp, Artificial Sequence
  Synthetic Polynucleotide
ttgcagaaccaccacctttaccgcgaacatt

SEQ ID NO: 23 - DNA, 27 bp, Artificial Sequence
  Synthetic Polynucleotide
ggtggtggttctgcaagcctgagcagc

SEQ ID NO: 24 - DNA, 31 bp, Artificial Sequence
  Synthetic Polynucleotide
agacctggattggatcttaggcaaccggctc

SEQ ID NO: 25 - DNA, 18 bp, Artificial Sequence
  Synthetic Polynucleotide
gatccaatccaggtctaa

SEQ ID NO: 26 - DNA, 18 bp, Artificial Sequence
  Synthetic Polynucleotide
catcatgcccatggtata

SEQ ID NO: 27 - DNA, 34 bp, Artificial Sequence
  Synthetic Polynucleotide
taccatgggcatgatggccgagatgttcaacggc

SEQ ID NO: 28 - DNA, 32 bp, Artificial Sequence
  Synthetic Polynucleotide
gatccggtgctgccggggatgatgggctcgac

SEQ ID NO: 29 - DNA, 30 bp, Artificial Sequence
  Synthetic Polynucleotide
ggcagcaccggatccgactttccgcagcaa

SEQ ID NO: 30 - DNA, 35 bp, Artificial Sequence
  Synthetic Polynucleotide
agttttgacgaaaggcttatttattacgctggatg

SEQ ID NO: 31 - DNA, 18 bp, Artificial Sequence
  Synthetic Polynucleotide
gcctttcgtcaaaactaa

SEQ ID NO: 32 - DNA, 27 bp, Artificial Sequence
  Synthetic Polynucleotide
tcgcaaaactaaagggaacaaaagctg

SEQ ID NO: 33 - DNA, 29 bp, Artificial Sequence
  Synthetic Polynucleotide
ttccattatcagagcagattgtactgaga

SEQ ID NO: 34 - DNA, 25 bp, Artificial Sequence
  Synthetic Polynucleotide
ctctgataatggaaggtcgggatga

SEQ ID NO: 35 - DNA, 25 bp, Artificial Sequence
  Synthetic Polynucleotide
ctttagttttgcgaaaccctatgct

SEQ ID NO: 36 - DNA, 38 bp, Artificial Sequence
  Synthetic Polynucleotide
gcgcggccgctttatatcatataattaagacacaaaag

SEQ ID NO: 37 - DNA, 26 bp, Artificial Sequence
  Synthetic Polynucleotide
gcgaattcataaagcagccgctacca

SEQ ID NO: 38 - DNA, 26 bp, Artificial Sequence
  Synthetic Polynucleotide
gcgaattcaaagcctggggtgcctaa

SEQ ID NO: 39 - DNA, 28 bp, Artificial Sequence
  Synthetic Polynucleotide
gcgcggccgcaggtggcacttttcgggg

SEQ ID NO: 40 - DNA, 25 bp, Artificial Sequence
  Synthetic Polynucleotide
aacaaaatggctctgttattagcag

SEQ ID NO: 41 - DNA, 25 bp, Artificial Sequence
  Synthetic Polynucleotide
ttttttatcagcttttgttcccttt

SEQ ID NO: 42 - DNA, 25 bp, Artificial Sequence
  Synthetic Polynucleotide
aagctgataaaaaacacgctttttc

SEQ ID NO: 43 - DNA, 25 bp, Artificial Sequence
  Synthetic Polynucleotide
agccattttgtttgtttatgtgtgt

SEQ ID NO: 44 - DNA, 25 bp, Artificial Sequence
  Synthetic Polynucleotide
tgtgctatggctctgttattagcag

SEQ ID NO: 45 - DNA, 25 bp, Artificial Sequence
  Synthetic Polynucleotide
attgttcagcttttgttccctttag

SEQ ID NO: 46 - DNA, 25 bp, Artificial Sequence
  Synthetic Polynucleotide
aagctgaacaatctgtttattaccc

SEQ ID NO: 47 - DNA, 22 bp, Artificial Sequence
  Synthetic Polynucleotide
agccatagcacagtgggcaatg

SEQ ID NO: 48 - DNA, 25 bp, Artificial Sequence
  Synthetic Polynucleotide
acaaaagctgaatgtttctactcct

SEQ ID NO: 49 - DNA, 25 bp, Artificial Sequence
  Synthetic Polynucleotide
tgttttttatgggacaggcaccgaa

SEQ ID NO: 50 - DNA, 19 bp, Artificial Sequence
  Synthetic Polynucleotide
ataaaaaacacgctttttc

SEQ ID NO: 51 - DNA, 19 bp, Artificial Sequence
  Synthetic Polynucleotide
cagcttttgttccctttag

SEQ ID NO: 52 - DNA, 24 bp, Artificial Sequence
  Synthetic Polynucleotide
gcggccgcctttcaagggtggggg

SEQ ID NO: 53 - DNA, 28 bp, Artificial Sequence
  Synthetic Polynucleotide
caggctttggctgaacaacagtctctcc

SEQ ID NO: 54 - DNA, 24 bp, Artificial Sequence
  Synthetic Polynucleotide
gttcagccaaagcctggggtgcct

SEQ ID NO: 55 - DNA, 24 bp, Artificial Sequence
  Synthetic Polynucleotide
cttgaaaggcggccgcaggtggca

SEQ ID NO: 56 - DNA, 18 bp, Artificial Sequence
  Synthetic Polynucleotide
taaagggaacaaaagctg

SEQ ID NO: 57 - DNA, 18 bp, Artificial Sequence
  Synthetic Polynucleotide
gcagattgtactgagagt

SEQ ID NO: 58 - DNA, 18 bp, Artificial Sequence
  Synthetic Polynucleotide
tctcagtacaatctgctc

SEQ ID NO: 59 - DNA, 18 bp, Artificial Sequence
  Synthetic Polynucleotide
gcttttgttccctttagt

SEQ ID NO: 60 - DNA, 25 bp, Artificial Sequence
  Synthetic Polynucleotide
tagaactatggaaaaaactgatctg

SEQ ID NO: 61 - DNA, 25 bp, Artificial Sequence
  Synthetic Polynucleotide
tcaatttttgaactttggccacgta

SEQ ID NO: 62 - DNA, 19 bp, Artificial Sequence
  Synthetic Polynucleotide
caaaaattgatttctgaag

SEQ ID NO: 63 - DNA, 18 bp, Artificial Sequence
  Synthetic Polynucleotide
catagttctagagctagc

SEQ ID NO: 64 - DNA, 15 bp, Artificial Sequence
  Synthetic Polynucleotide
ggcagcaccggatcc

SEQ ID NO: 65 - DNA, 19 bp, Artificial Sequence
  Synthetic Polynucleotide
cattttgtttgtttatgtg

SEQ ID NO: 66 - DNA, 25 bp, Artificial Sequence
  Synthetic Polynucleotide
aaacaaaatggactccttcatcttc

SEQ ID NO: 67 - DNA, 25 bp, Artificial Sequence
  Synthetic Polynucleotide
gacaagatttcgtccaacttcaatc

SEQ ID NO: 68 - DNA, 25 bp, Artificial Sequence
  Synthetic Polynucleotide
tggacgaaatcttgtcctccttgat

SEQ ID NO: 69 - DNA, 25 bp, Artificial Sequence
  Synthetic Polynucleotide
cggtgctgccagatcttgggaacaa

SEQ ID NO: 70 - DNA, 25 bp, Artificial Sequence
  Synthetic Polynucleotide
aaagctagtttatcattatcaatac

SEQ ID NO: 71 - DNA, 25 bp, Artificial Sequence
  Synthetic Polynucleotide
tatcatatcaaattaaagccttcga

SEQ ID NO: 72 - DNA, 25 bp, Artificial Sequence
  Synthetic Polynucleotide
ttaatttgatatgatacacggtcca

SEQ ID NO: 73 - DNA, 25 bp, Artificial Sequence
  Synthetic Polynucleotide
atcggcatagcttcaaaatgtttct

SEQ ID NO: 74 - DNA, 25 bp, Artificial Sequence
  Synthetic Polynucleotide
gaagctatgccgatttcggcctatt

SEQ ID NO: 75 - DNA, 24 bp, Artificial Sequence
  Synthetic Polynucleotide
gataaactagcttttgttcccttt

SEQ ID NO: 76 - DNA, 25 bp, Artificial Sequence
  Synthetic Polynucleotide
aaacaaaatggaaaaaactgatctg

SEQ ID NO: 77 - DNA, 25 bp, Artificial Sequence
  Synthetic Polynucleotide
taagcttttaaactttggccacgta

SEQ ID NO: 78 - DNA, 15 bp, Artificial Sequence
  Synthetic Polynucleotide
taaaagcttatcgat

SEQ ID NO: 79 - DNA, 25 bp, Artificial Sequence
  Synthetic Polynucleotide
tagaactatggactccttcatcttc

SEQ ID NO: 80 - DNA, 25 bp, Artificial Sequence
  Synthetic Polynucleotide
tcaatttttgccaaatatcccgtaa

SEQ ID NO: 81 - DNA, 25 bp, Artificial Sequence
  Synthetic Polynucleotide
caaaagctaatgtttctactccttt

SEQ ID NO: 82 - DNA, 25 bp, Artificial Sequence
  Synthetic Polynucleotide
gaaacattagcttttgttcccttta

SEQ ID NO: 83 - DNA, 34 bp, Artificial Sequence
  Synthetic Polynucleotide
ataaacaaacaaaatggactcttttccattattg

SEQ ID NO: 84 - DNA, 29 bp, Artificial Sequence
  Synthetic Polynucleotide
gatccggtgctgccggacttgacgattgg

SEQ ID NO: 85 - DNA, 32 bp, Artificial Sequence
  Synthetic Polynucleotide
ggcagcaccggatccgaaccatcctctaaaaa

SEQ ID NO: 86 - DNA, 34 bp, Artificial Sequence
  Synthetic Polynucleotide
gatggtgatgatgatgccagacatctctcaagta

SEQ ID NO: 87 - DNA, 18 bp, Artificial Sequence
  Synthetic Polynucleotide
catcatcatcaccatcac

SEQ ID NO: 88 - DNA, 19 bp, Artificial Sequence
  Synthetic Polynucleotide
cattttgtttgtttatgtg

SEQ ID NO: 89 - DNA, 32 bp, Artificial Sequence
  Synthetic Polynucleotide
ataaacaaacaaaatgcaattcttctccttgg

SEQ ID NO: 90 - DNA, 29 bp, Artificial Sequence
  Synthetic Polynucleotide
gatccggtgctgccttctctggatggttg

SEQ ID NO: 91 - DNA, 30 bp, Artificial Sequence
  Synthetic Polynucleotide
ggcagcaccggatccacttctgccttgtat

SEQ ID NO: 92 - DNA, 31 bp, Artificial Sequence
  Synthetic Polynucleotide
gatggtgatgatgatgccagacatctctcaa

SEQ ID NO: 93 - DNA, 25 bp, Artificial Sequence
  Synthetic Polynucleotide
tagaactatgaccacagttttcgct

SEQ ID NO: 94 - DNA, 25 bp, Artificial Sequence
  Synthetic Polynucleotide
tcaatttttgccccctgactttact

SEQ ID NO: 95 - DNA, 725 bp, Artificial Sequence
  Synthetic Polynucleotide
aatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaattttttagagattactacatattccaacaagaccttcgcaggaaagtatacctaaactaattaaagaaatctccgaagttcgcatttcattgaacggctcaattaatctttgtaaatatgagcgtttttacgttcacattgcctttttttttatgtatttaccttgcatttttgtgctaaaaggcgtcacgtttttttccgccgcagccgcccggaaatgaaaagtatgacccccgctagaccaaaaatacttttgtgttattggaggatcgcaatccctttcagtggaattattagaatgaccactactccttctaatcaaacacgcggaaatagccgccaaaagacagattttattccaaatgcgggtaactatttgtataatatgtttacatattgagcccgtttaggaaagtgcaagttcaaggcactaatcaaaaaaggagatttgtaaatatagcgaccgaatcaggaaaaggtcaacaacgaagttcgcgatatggatgaacttcggtgcctgtccc

SEQ ID NO: 96 - DNA, 1320 bp, Artificial Sequence
  Synthetic Polynucleotide
atggaaaaaactgatctgcatgtcaatctgattgaaaaagttatggttggtcctagccctccactgccgaaaactaccctgcaactgtccagcattgataacctgccgggtgtacgcggtagcattttcaacgcactgctgatctataacgcaagcccgagcccgaccatgatctctgcagatccagcaaaaccgatccgcgaagcgctggcgaaaattctggtttactacccgccatttgcaggccgcctgcgtgaaaccgaaaacggtgatctggaagttgagtgcaccggtgaaggtgcgatgttcctggaagcgatggcggacaacgaactgagcgttctgggtgactttgacgattccaacccgtcttttcagcaactgctgttttccctgcctctggataccaactttaaagatctgtccctgctggttgttcaggtgacccgctttacctgtggcggtttcgtcgtcggcgttagcttccaccacggcgtttgcgacggccgtggcgccgcacagtttctgaaaggcctggctgaaatggcacgtggtgaggtgaagctgtctctggaaccgatttggaaccgcgaactggttaaactggatgacccgaaatacctgcagttctttcacttcgagttcctgcgtgcgccgagcatcgtagagaaaatcgttcagacctatttcatcattgatttcgagacgatcaactatatcaaacagtctgtaatggaagagtgcaaggagttctgctcttccttcgaagtggcttccgctatgacttggatcgcgcgtactcgtgcgttccagattccggaaagcgaatacgtgaagatcctgttcggcatggacatgcgtaactctttcaacccgccgctgccgtccggctattacggtaacagcatcggcacggcgtgtgctgtggacaacgttcaggatctgctgtctggttctctgctgcgtgctatcatgattatcaagaaatccaaagtctccctgaacgacaatttcaaatctcgcgcggtggtaaaaccgtccgaactggacgtgaacatgaatcacgaaaacgtagtcgccttcgctgactggtcccgtctgggtttcgacgaagtagacttcggctggggtaatgctgtatctgtttctccggtgcagcagcagtctgctctggccatgcaaaactacttcctgttcctgaaaccgtctaaaaacaaaccagatggtatcaaaatcctgatgtttctgccgctgtctaagatgaaatctttcaaaatcgaaatggaagccatgatgaagaaatacgtggccaaagtttaa

SEQ ID NO: 97 - DNA, 1494 bp, Artificial Sequence
  Synthetic Polynucleotide
atggactccttcatcttcttgagatccattggtactaagttcggtcaattggaatcttccccagctattttgtctttgactttggctccaattttggccatcatcttgttgttgttattcagatacaaccacagatcctctgttaagttgccaccaggtaaattgggttttccattgattggtgaaaccatccaattattgagaaccttgagatctgaaaccccacaaaagttcttcgatgacagattgaaaaagtttggtccagtctacatgacctcattgataggtcatccaactgttgttttgtgtggtccagctggtaacaaattggttttgtctaacgaagataagttggttgaaatggaaggtccaaagtctttcatgaagttgatcggtgaagattctatcgttgctaagagaggtgaagatcacagaattttgagaactgctttggctagatttttgggtgctcaagctttacaaaactacttgggtagaatgtcctccgaaattggtcatcattttaacgaaaagtggaagggtaaggatgaagttaaggttttgccattggtcagaggtttgattttctctattgcttccaccttgttcttcgacgttaatgatggtcatcaacaaaagcaattgcaccacttgttggaaaccattttggttggttctttgtccgttccattggattttccaggtactagatacagaaagggtttacaagctagattgaagttggacgaaatcttgtcctccttgattaagcgtagaagaagagatttgagatccggtattgcttccgatgatcaagatttgttgtctgtcttgttgaccttcagagatgaaaagggtaactctttgaccgatcaaggtatcttggataacttctctgctatgttccatgcttcttacgatacaactgttgctccaatggctttgatcttcaagttgttgtactctaacccagaataccacgaaaaggttttccaagaacaattggaaatcatcggtaacaagaaagaaggtgaagaaatctcctggaaggacttgaaatctatgaagtacacttggcaagccgtccaagaatctttgagaatgtatccaccagttttcggtattttcagaaaggccattaccgatatccattacgatggttacactattccaaagggttggagagttttgtgttctccatataccactcacttgagagaagaatactttccagaaccagaagaattcagaccatccagatttgaagatgaaggtagacatgttactccatacacttacgttccatttggtggtggtttgagaacttgtccaggttgggaattttctaagatcgaaatcttgttgttcgtccaccacttcgttaagaacttctcatcttacattccagtcgacccaaacgaaaaagttttgtctgatccattgccaccattaccagctaatggtttctctattaagttgttcccaagatcttaa

SEQ ID NO: 98 - DNA, 1788 bp, Artificial Sequence
  Synthetic Polynucleotide
atgagcctggcatttaatccggcagcaaccgcatttagcggtaatggtgcacgtagccgtcgtgaaaactttccggttaaacatgttaccgttcgtggttttccgatgattaccaataaaagcagctttgccgttaaatgcaatctgaccaccaccgatctgatgggcaaaattgcagaaaaattcaaaggcgaggatagcaattttccggcagccgcagcagttcagcctgcagcagatatgccgagcaatctgtgtattattgataccctgcagcgtctgggtgttgatcgttattttcgtagcgaaattgataccatcctggaagatacctatcgtctgtggcagcgtaaagaacgtgcaatttttagcgataccgcaattcatgcaatggcatttcgtctgctgcgtgttaaaggttatgaagttagcagcgaagaactggcaccgtatgcagatcaagaacatgtggatctgcagaccattgaagttgcaaccgttattgaactgtatcgtgcagcacaagaacgtaccggtgaagatgaaagcagcctgaaaaaactgcatgcatggaccaccacatttctgaaacagaaactgctgaccaatagcatcccggataaaaaactgcacaaactggtggaatactatctgaaaaactttcacggcattctggatcgtatgggtgttcgtcagaatctggatctgtacgatattagttattaccgtaccagcaaagcagccaatcgttttagtaatctgtgctccgaagattttctggcatttgcacgtcaggattttaacatttgtcaggcacagcatcagaaagaactgcagcagctgcaacgttggtatgccgattgtaaactggataccctgaaatatggtcgtgatgttgttcgtgttgcaaattttctgaccagcgcaattattggtgatccggaactgagtgatgttcgtattgtttttgcacagcatattgttctggtgacccgcatcgatgatttttttgatcatcgtggtagccgtgaagagagctacaaaattctggaactgatcaaagaatggaaagaaaaaccggcagcagaatatggtagcgaagaagttgaaattctgttcaccgcagtgtataataccgtgaatgaactggcagaacgtgcccatgttgaacagggtcgtagcgttaaagatttcctgattaaactgtgggtgcagatcctgagcatctttaaacgtgagctggatacctggtcagatgataccgcactgaccctggatgattatctgagcgcaagctgggttagcattggttgtcgtatttgtattctgatgtccatgcagttcattggcatcaaactgtcagatgaaatgctgctgagcgaagaatgtattgatctgtgtcgtcatgttagcatggtggatcgcctgctgaatgatgttcagacctttgaaaaagaacgcaaagagaataccggtaatagcgttaccctgctgctggcagcaaataaagatgatagcagttttaccgaagaagaggcaattcgtattgcaaaagaaatggccgaatgtaatcgtcgtcagctgatgcagattgtgtataaaaccggtacaatttttccgcgtcagtgcaaagatatgtttctgaaagtttgccgcattgggtgttatctgtatgcaagcggtgatgaatttaccagtccgcagcagatgatggaagatatgaaaagcctggtttatgaaccgctgaccattcatccgctggttgcaaataatgttcgcggtaaataa

SEQ ID NO: 99 - DNA, 2382 bp, Artificial Sequence
  Synthetic Polynucleotide
atggcaagcctgagcagcaccattctgagccgtagtccggcagcacgtcgtcgtattacaccggcaagcgcaaaactgcatcgtccggaatgttttgcaaccagcgcatggatgggtagcagcagcaaaaatctgagcctgagctatcagctgaaccacaaaaaaatcagcgttgcaaccgttgatgcaccgcaggttcatgatcacgatggcaccaccgttcatcagggtcatgatgcagttaaaaacattgaagatccgatcgaatatatccgtaccctgctgcgtaccaccggtgatggtcgtattagcgttagcccgtatgataccgcatgggttgcaatgattaaagatgttgaaggtcgtgatggtccgcagtttccgagcagcctggaatggattgttcagaatcagctggaagatggtagctggggtgatcagaaactgttttgtgtttatgatcgtctggtgaataccattgcatgtgttgttgcactgcgtagctggaatgttcatgcacataaagttaaacgtggcgtgacctatatcaaagaaaacgtggataaactgatggaaggcaacgaagaacatatgacctgtggttttgaagttgtttttccggcactgctgcagaaagcaaaaagcctgggtatcgaagatctgccgtatgattcaccggcagttcaagaggtttatcatgttcgtgaacaaaaactgaaacgcattccgctggaaatcatgcataaaattccgaccagtctgctgtttagcctggaaggtctggaaaatctggattgggacaaactgctgaaactgcagagcgcagatggtagttttctgaccagcccgagcagtaccgcatttgcatttatgcagaccaaagatgagaaatgctatcagttcatcaaaaacaccatcgacacctttaatggtggtgcaccgcatacctatccggttgatgtttttggtcgtctgtgggcaattgatcgcctgcagcgtctgggtattagccgtttttttgaaccggaaattgcagattgtctgagccacattcacaaattctggaccgataaaggtgtttttagcggtcgtgaaagcgaattttgcgatattgatgataccagtatgggtatgcgtctgatgcgtatgcatggttatgatgttgatccgaatgttctgcgcaactttaaacagaaagatggcaaatttagctgctatggtggtcagatgattgaaagcccgagcccgatttataacctgtatcgtgcaagccagctgcgttttccgggtgaagaaattctggaagatgccaaacgttttgcctatgatttcctgaaagaaaaactggccaataaccagatcctggataaatgggttattagcaaacatctgccggatgaaattaaactgggcctggaaatgccgtggctggcaaccctgcctcgtgttgaagcaaaatactatattcagtattatgccggtagcggtgatgtgtggattggtaaaaccctgtatcgcatgccggaaattagcaatgatacctatcatgatctggccaaaaccgattttaaacgttgtcaggcaaaacaccagtttgagtggctgtatatgcaagaatggtatgaaagctgcggcattgaagaatttggcattagccgtaaagatctgctgctgagctattttctggcaaccgcaagcatctttgaactggaacgtaccaatgaacgtattgcatgggccaaaagccagattattgcaaaaatgatcaccagcttttttaacaaagaaaccacgagcgaagaagataaacgcgcactgctgaatgaactgggtaacattaatggtctgaatgataccaatggtgcaggtcgtgaaggtggtgcgggtagcattgcactggccaccctgacccagtttctggaaggttttgatcgttatacccgtcaccagctgaaaaatgcatggtcagtttggctgacccagctgcagcatggtgaagcagatgatgcagaactgctgaccaataccctgaatatttgtgccggtcatattgcctttcgcgaagaaatcctggcacacaatgaatataaagcactgagcaatctgacgagcaaaatttgtcgccagctgagctttattcagagcgaaaaagaaatgggtgtggaaggtgaaattgccgcaaaaagcagcattaaaaacaaagaactggaagaggatatgcagatgctggttaaactggttctggagaaatatggtggtattgaccgcaatatcaaaaaagcatttctggcagttgccaaaacctattattaccgtgcatatcatgcagccgataccattgatacccatatgttcaaagttctgtttgagccggttgcctaa

SEQ ID NO: 100 - DNA, 1488 bp, Artificial Sequence
  Synthetic Polynucleotide
atggactcttttccattattggctgccttgtttttcattgctgctactattaccttcttgtccttccgtagaagaagaaatttgccaccaggtccatttccatatccaatcgttggtaatatgttgcaattgggtgctaacccacatcaagtttttgctaagttgtctaagagatacggtccattgatgtccattcatttgggttccttgtacaccgttatagtctcttcaccagaaatggccaaagaaatcttgcatagacatggtcaagttttctccggtagaactattgctcaagctgttcatgcttgtgatcacgataagatttctatgggttttttgccagttgcctctgaatggagagatatgagaaagatctgcaaagaacaaatgttctccaatcaatccatggaagcttctcaaggtttgagaagacaaaagttgcaacaattattggaccacgtccaaaagtgttctgattctggtagagctgttgatattagagaagctgctttcattaccaccttgaatttgatgtctgctaccttgttttcttcacaagctaccgaatttgattccaaggctaccatggaattcaaagaaattattgaaggtgttgccaccatcgttggtgttccaaattttgctgattacttcccaatcttaagaccattcgatccacaaggtgttaagagaagagctgatgtttttttcggtaagttgttggccaagatcgaaggttatttgaacgaaagattggaatccaagagagctaatccaaacgctccaaagaaggatgatttcttggaaatcgttgtcgatatcatccaagccaacgaattcaagttgaaaacccatcatttcacccacttgatgttggatttgtttgttggtggttctgataccaacaccacttctattgaatgggctatgtctgaattggttatgaacccagataagatggctagattgaaggctgaattgaaatctgttgctggtgacgaaaagatcgttgatgaatctgctatgccaaagttgccatacttgcaagctgttatcaaagaagtcatgagaattcatccacctggtcctttgttgttaccaagaaaagctgaatccgatcaagaagtcaacggttacttaattccaaagggtactcaaatcttgattaacgcttacgccattggtagagatccatctatttggactgatccagaaacttttgacccagaaagattcttggacaacaagatcgatttcaagggtcaagactacgaattattgccatttggttcaggtagaagagtttgtccaggtatgccattggctactagaatattgcatatggctactgctactttggttcacaatttcgattggaagttggaagatgattctactgctgctgctgatcatgctggtgaattatttggtgttgctgttagaagagcagtcccattgagaattattccaatcgtcaagtcctaa

SEQ ID NO: 101 - DNA, 2118 bp, Artificial Sequence
  Synthetic Polynucleotide
atggaaccatcctctaaaaagttgtccccattggatttcattaccgccattttgaagggtgatattgaaggtgttgctccaagaggtgttgcagctatgttgatggaaaacagagatttggctatggttttgactacctctgttgctgttttgattggttgcgttgttgttttggcttggagaagaactgctggttctgctggtaaaaaacaattgcaaccaccaaagttggttgttccaaaaccagctgctgaacctgaagaagctgaagacgaaaaaactaaggtcagtgttttcttcggtactcaaactggtactgctgaaggttttgctaaagcttttgccgaagaagctaaagctagatatccacaagctaagttcaaggttatcgatttggatgattacgctgccgatgatgatgaatacgaagaaaagttgaagaaagaatccttggccttcttcttcttggcttcttatggtgatggtgaacctactgataatgctgctagattttacaagtggttcaccgaaggtaaggatagagaagattggttgaagaacttgcaatacggtgtttttggtttgggtaacagacaatacgaacacttcaacaagattgccatcgttgtcgatgatttgattactgaacaaggtggtaagaagttggttccagttggtttaggtgatgatgatcaatgcatcgaagatgatttttccgcttggagagaattggtttggccagaattggataagttgttgagaaatgaagatgatgctactgttgctactccatataccgctgttgttttacaatacagagttgtcttgcacgatcaaactgatggtttgatcacagaaaatggttctccaaatggtcatgctaacggtaacactatctatgatgctcaacatccatgtagagctaacgttgctgttagaagagaattgcatactccagcttcagatagatcttgtacccatttggaattcgatacttcaggtactggtttggtttacgaaactggtgatcatgttggtgtttactgcgaaaacttgttggaaaatgtcgaagaagccgaaaagttattgaacttgtctccacaaacctacttctccgttcatactgataacgaagatggtactccattgtctggttcttcattgccaccaccatttccaccatgtactttgagaactgctttgactaagtacgccgatttgatttctatgccaaagaagtctgttttggttgctttggctgaatacgcctctaatcaatcagaagctgatagattgagatacttggcttcaccagatggtaaagaagaatacgcccaatatatcgttgcctcccaaagatcattattggaagttatggctgaattcccatctgctaaaccaccattgggtgttttttttgctgctattgctcctagattgcaacctagattctactccatttcttcctccccaaaaattgctccaactagagttcatgttacctgtgctttggtttatgataagactccaactggtagaatccataagggtatttgttctacctggattaagaacgctgttccattggaagaatcttcagattgctcttgggctccaattttcatcagaaactctaactttaagttgccagccgatccaaaggttccaattatcatggttggtccaggtacaggtttagctccttttagaggtttcttacaagaaagattggccttgaaagaatctggtgctgaattgggtccagctattttgttttttggttgtagaaacagaaagatggacttcatatacgaagatgaattgaactccttcgttaaggttggtgccatttctgaattgatcgttgctttttctagagaaggtccagccaaagaatacgttcaacataagatgtctcaaagagcctccgatatttggaagatgatatctgatggtggttacatgtacgtttgtggtgatgctaaaggtatggctagagatgttcatagaaccttgcataccattgctcaagaacaaggttctttgtcatcttctgaagcagaaggtatggtcaaaaacttgcaaactactggtagatacttgagagatgtctggtaa

SEQ ID NO: 102 - DNA, 1770 bp, Artificial Sequence
  Synthetic Polynucleotide
atggccgagatgttcaacggcaactcttctaacgacggatcttcttgcatgcccgtgaaggacgccctgcgacgaaccggcaaccaccaccccaacctgtggaccgacgacttcatccagtctctgaactctccctactctgactcttcttaccacaagcaccgagagatcctgatcgacgagatccgagacatgttctctaacggcgagggcgacgagttcggcgtgctcgagaacatctggttcgtggacgtggtgcagcgactgggcatcgaccgacacttccaggaggagatcaagaccgccctggactacatctacaagttctggaaccacgactctatcttcggcgacctgaacatggtggccctgggcttccgaatcctgcgactgaaccgatacgtggcctcttctgacgtgttcaagaagttcaagggcgaggagggccagttctctggcttcgagtcctctgaccaggacgctaagctcgaaatgatgctgaacctgtacaaggcctctgagctggacttccccgacgaggacatcctgaaggaggcccgagccttcgcctctatgtacctgaagcacgtgatcaaggagtacggcgacatccaggagtctaagaaccccctgctgatggagatcgagtacaccttcaagtacccctggcgatgccgactgccccgactcgaggcctggaacttcatccacatcatgcgacagcaggactgcaacatctctctggccaacaacctctacaagatccccaagatctacatgaagaagatcctcgagctggccatcctggacttcaacatcctgcagtctcagcaccagcacgagatgaagctgatctctacctggtggaagaactcttctgctatccagctggacttcttccgacaccgacacatcgagtcttacttttggtgggcctcgcccctgttcgagcccgagttctctacctgccgaatcaactgcaccaagctgtctaccaagatgttcctgctggacgacatctacgacacctacggcaccgtcgaggagctgaagcccttcaccaccaccctgacccgatgggacgtgtctaccgtggacaaccaccccgactacatgaagatcgccttcaacttctcttacgagatctacaaggagatcgcctctgaggccgagcgaaagcacggccccttcgtgtacaagtacctgcagtcttgctggaagtcttacatcgaggcctacatgcaggaggccgagtggatcgcctctaaccacatccccggcttcgacgagtacctgatgaacggcgtgaagtcctctggcatgcgaatcctgatgatccacgccctgatcctgatggacacccccctgtctgacgagattctcgagcagctggacatcccctcgtctaagtctcaggccctgctgtctctgatcacccgactggtggacgacgtgaaggacttcgaggacgagcaggcccacggcgagatggcctcttctatcgagtgctacatgaaggacaaccacggctctacccgagaggacgccctgaactacctgaagatccgaatcgagtcttgcgtgcaggagctgaacaaggagctgctcgagccctctaacatgcacggatctttccgaaacctgtacctgaacgtgggaatgcgagtgattttcttcatgctgaacgacggcgacctgttcacccactctaaccgaaaggagatccaggacgccatcaccaagttcttcgtcgagcccatcatcccctga

SEQ ID NO: 103 - DNA, 1509 bp, Artificial Sequence
  Synthetic Polynucleotide
atgcaattcttctccttggtttccatcttcttgttcttgtcctttttgtttttgttgagaaagtggaagaactccaactcccaatctaaaaagttgccaccaggtccatggaaattgccattattgggttctatgttgcatatggttggtggtttgccacatcatgttttgagagatttggctaaaaagtacggtccattgatgcacttgcaattgggtgaagtttctgctgttgttgttacttctccagatatggccaaagaagttttgaaaacccacgatattgctttcgcttctagaccaaagttgttggctccagaaatcgtttgttacaacagatctgatattgccttctgtccatacggtgattattggagacaaatgagaaagatctgcgtcttggaagttttgtctgctaagaacgtcagatccttcagttccattagaagagatgaagtcttgagattggtcaacttcgttagatcttctacttccgaaccagttaacttcaccgaaagattattcttgttcacctcttctatgacctgtagatctgcttttggtaaggttttcaaagaacaagaaaccttcatccaattgatcaaagaagtcattggtttggctggtggttttgatgttgctgatattttcccatccttgaagttcttgcatgtcttgactggtatggaaggtaagattatgaaggcccatcataaggttgatgccatcgttgaagatgttatcaacgaacacaaaaagaacttggctatgggtaagactaatggtgctttgggtggtgaagatttgatcgatgttttgttaagattgatgaacgatggtggtttacaattcccaatcaccaacgataacattaaggccatcatcttcgatatgtttgctgctggtactgaaacttcttcctctactttggtttgggctatggttcaaatgatgagaaacccaactattttggctaaggctcaagctgaagttagagaagcttttaagggtaaagaaactttcgacgaaaacgacgtcgaagaattgaagtacttgaagttggttatcaaagaaacattgagattgcacccaccagttcctttgttggttccaagagaatgtagagaagaaaccgaaatcaacggttacaccattccagttaagaccaaggttatggttaatgtttgggctttgggtagagatccaaagtattgggatgatgctgataacttcaagccagaaagattcgaacaatgctccgttgactttatcggtaacaacttcgaatacttgccatttggtggtggtagaagaatttgtccaggtatttctttcggtttggccaatgtttatttgccattggctcaattgttgtaccacttcgattggaaattaccaacaggtatggaacctaaggatttggatttgactgaattggtcggtattaccattgccagaaagtccgatttgatgttagttgctactccataccaaccatccagagaataa

SEQ ID NO: 104 - DNA, 2079 bp, Artificial Sequence
  Synthetic Polynucleotide
atgacttctgccttgtatgcctctgatttgttcaagcaattgaagtccattatgggtactgactcattgtccgatgatgttgttttggttattgctactacctccttggctttggttgctggttttgttgttttattgtggaaaaagaccaccgccgatagatctggtgaattgaaaccattgatgatcccaaagtctttgatggccaaagatgaagatgatgatttggatttgggttccggtaagactagagtttctattttcttcggtactcaaaccggtactgctgaaggttttgctaaagctttgtctgaagaaatcaaggccagatacgaaaaagctgccgttaaggttatagatttggatgattatgctgccgatgacgaccaatacgaagaaaagttgaagaaagaaaccttggccttcttctgtgttgctacttatggtgatggtgaacctactgataatgctgctagattttacaagtggttcactgaagaaaacgaaagagacatcaagttgcaacaattggcttacggtgtttttgctttgggtaacagacaatacgaacacttcaacaagatcggtatcgttttggatgaagaattgtgtaagaagggtgccaagagattgattgaagttggtttgggtgatgatgaccaatccatcgaagatgattttaacgcctggaaagaatccttgtggtctgaattggataagttgttgaaggacgaagatgacaaatctgttgctacaccatacactgctgttatcccagaatatagagttgttacccatgatccaagattcaccactcaaaagtctatggaatctaacgttgctaacggtaacaccaccatcgatattcatcatccatgtagagttgatgtcgccgtccaaaaagaattgcatactcatgaatctgacagatcctgcatccatttggaattcgatatttccagaaccggtattacttacgaaaccggtgatcatgttggtgtttacgctgaaaatcacgttgaaatcgttgaagaagccggtaagttgttaggtcattccttagatttggttttctccatccatgccgacaaagaagatggttctccattggaatctgctgttccaccaccatttccaggtccatgtactttgggtactggtttggctagatatgctgacttgttgaatccaccaagaaagtctgctttagttgctttggctgcttatgctactgaaccatctgaagccgaaaaattgaaacatttgacttccccagatggtaaggacgaatattctcaatggatagttgcctcccaaagatccttgttggaagttatggctgcttttccatctgctaaaccaccattgggtgttttttttgctgctattgctccaagattgcaacctagatattactccatttcctccagtccaagattagctccatcaagagttcatgttacatccgctttggtttatggtccaactccaactggtagaattcataagggtgtttgttctacctggatgaagaacgctgttccagctgaaaaatctcatgaatgttctggtgccccaattttcattagagcttctaatttcaagttgccatccaacccatctactccaatagttatggttggtccaggtacaggtttagctccttttagaggtttcttacaagaaagaatggccttgaaagaagatggtgaagaattgggttcctccttgttgttttttggttgcagaaacagacaaatggatttcatctatgaagatgaattgaacaacttcgtcgaccaaggtgttatctccgaattgattatggccttttctagagaaggtgctcaaaaagaatacgtccaacacaagatgatggaaaaagccgctcaagtttgggacttgatcaaagaagaaggttacttgtacgtttgcggtgatgctaaaggtatggctagagatgttcatagaacattgcataccatcgttcaagaacaagaaggtgtctcatcttctgaagctgaagctatcgttaagaagttgcaaactgaaggtagatacttgagagatgtctggtaa

SEQ ID NO: 105 - protein, 5 aa, artificial sequence
  synthetic peptide
GSTST